SEQUENCE LISTING (65 sequences)

SEQ ID NO: 1 - protein, 578 aa, Punctularia strigosozonata
  REGION 1..578: note = alpha 1,4-glucosidase (glucoamylase) Pstr_GA_0048
MLSSLIVSGLLASGVCASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFYDGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 2 - DNA, 1753 bp, Punctularia strigosozonata
  misc_feature 1..1753: note = alpha 1,4-glucosidase (glucoamylase) Pstr_GA_0048
  source 1..1753: mol_type = genomic DNA
ggtctcgaatgttgtcctctttgatcgtttccggtttgttggcttctggtgtctgtgcctcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaagttgcctcttcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagatgttttctacgacggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaggagacc

SEQ ID NO: 3 - protein, 580 aa, Punctularia strigosozonata
  REGION 1..580: note = alpha 1,4-glucosidase (glucoamylase) Pstr_GA_0009
MRFPSIFTAVLFAASSALASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEINSSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFMGGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 4 - DNA, 1759 bp, Punctularia strigosozonata
  misc_feature 1..1759: note = alpha 1,4-glucosidase (glucoamylase) Pstr_GA_0009
  source 1..1759: mol_type = genomic DNA
ggtctcgaatgagattcccatccatcttcaccgccgtcttgttcgctgcttcttctgctttggcttcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaaatcaactcctcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagatgttttcatgggtggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaggagacc

SEQ ID NO: 5 - protein, 580 aa, Punctularia strigosozonata
  REGION 1..580: note = alpha 1,4-glucosidase (glucoamylase) Pstr_GA_0010
MRFPSIFTAVLFAASSALASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEINSSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDSFMGGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 6 - DNA, 1759 bp, Punctularia strigosozonata
  misc_feature 1..1759: note = alpha 1,4-glucosidase (glucoamylase) Pstr_GA_0010
  source 1..1759: mol_type = genomic DNA
ggtctcgaatgagattcccatccatcttcaccgccgtcttgttcgctgcttcttctgctttggcttcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaaatcaactcctcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagattctttcatgggtggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaggagacc

SEQ ID NO: 7 - protein, 580 aa, Punctularia strigosozonata
  REGION 1..580: note = alpha 1,4-glucosidase (glucoamylase) Pstr_GA_0033
MRFPSIFTAVLFAASSALASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFMGGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 8 - DNA, 1759 bp, Punctularia strigosozonata
  misc_feature 1..1759: note = alpha 1,4-glucosidase (glucoamylase) Pstr_GA_0033
  source 1..1759: mol_type = genomic DNA
ggtctcgaatgagattcccatccatcttcaccgccgtcttgttcgctgcttcttctgctttggcttcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaagttgcctcttcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagatgttttcatgggtggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaggagacc

SEQ ID NO: 9 - protein, 580 aa, Punctularia strigosozonata
  REGION 1..580: note = alpha 1,4-glucosidase (glucoamylase) Pstr_GA_0034
MRFPSIFTAVLFAASSALASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDSFMGGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 10 - DNA, 1759 bp, Punctularia strigosozonata
  misc_feature 1..1759: note = alpha 1,4-glucosidase (glucoamylase) Pstr_GA_0034
  source 1..1759: mol_type = genomic DNA
ggtctcgaatgagattcccatccatcttcaccgccgtcttgttcgctgcttcttctgctttggcttcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaagttgcctcttcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagattctttcatgggtggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaggagacc

SEQ ID NO: 11 - protein, 561 aa, Punctularia strigosozonata
  REGION 1..561: note = Punctularia strigosozonata glucoamylase
SSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFYDGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 12 - protein, 578 aa, Punctularia strigosozonata; identical to SEQ ID NO: 1
  REGION 1..578: note = Punctularia strigosozonata glucoamylase with native signalsequence FGA09

SEQ ID NO: 13 - protein, 580 aa, synthetic construct
  REGION 1..580: note = Punctularia strigosozonata glucoamylase with Sc alpha matingfactor signal sequence
MRFPSIFTAVLFAASSALASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFYDGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 14 - protein, 619 aa, Myceliophthora sepedonium
  REGION 1..619: note = Myceliophthora sepedonium (Corynascus sepedonium) glucoamylase(mature)
RPGAIPRSQRGGAISKRAVDSYIETETPIAWEKLLCNIGPDGCAVSGAAAGAVIASPSKSDPDYWYTWTRDAGLVLTGIVDSLAHNYSASLQTNIQNYIIAQAKLQGVGNPSGGLSDGAGLGEPKFMVDLTEFTGDWGRPQRDGPPLRAIALIRYAKWLVANGYKDTANELVWPVIQNDLAYAAQYWNETGFDLWEEVPGSSFFTIASTHRALVEGAALAAQLDTECRACITVAPQVLCFLQTFWNPSGGYVVSNINGGEGRSGKDLNSILASAHTFDPAIGCDSVTFQPCSDKALANHKAYVDSFREIYGINSGIAKGKAVAVGRYSEDVYYNGNPWYLANFGAAEQLYDAIYVWKEQGSIEVTDLSLPFFQDLLSDISTGTYDSSSSTYQEILDAVSAYADGFIDVAAQYTPSDGSLAEQFERDSGNPISAADLTWSYSAFLSATDRRAGIVPAGWSAENGKTLPDSCEAIQVAGTYTQASPTAFPPNQTPNPSAETPETPFPSSCADANEVYVTFKGKVTTQWGESVKVVGSTPELGSWDVKKAVPLSASAYTESNPLWKITVPMKAGQAVQYKFIRVNGDGKAQWESDPNRTFEVGAAGKADGGCSSQTVEGTWR

SEQ ID NO: 15 - protein, 622 aa, Aspergillus niger
  REGION 1..622: note = Aspergillus niger glucoamylase (mature)
NVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 16 - protein, 612 aa, Trichoderma reesei
  REGION 1..612: note = T. reesei glucoamylase (mature)
RPGSSGLSDVTKRSVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 17 - protein, 535 aa, Botryotinia fuckeliana
  REGION 1..535: note = Botryotinia fuckeliana glucoamylase (mature)
KNLEHPKLSTRTTGTIDDYLTAQSPISLQGILNNIGPDGSKAQGASAGIVVASPSTVDPNYFYTWTRDSALTFKYLIDTGNSSLQSLIEDYVTAQAKLQTVENPSGDLATGAGLAEPKYYTNETAFLGEWGRPQRDGPALRAIALISFGNDRLTLGQNDTVKEIIWPIVRNDLAYVSQYWNFSGFDLWEEINGSSFFTTAVQHRALVEGAKFATALGESCNDCEEQAPEVLCMLQDYWNGASINSNIQVQSSTYDRSGLDCNSILTSIHTFDPDQAVGCDSTTFQPCSDRALANHKVLVDSFRSIYTINSNASTGQAAAIGRYAEDVYQGGNPWYICTYAAAEQLYDALWTYNTTESITITDISQDFFTDLVPGIETGTFASDSSQFKDIVKAMQDYADGFVSIAETYTPEDGALAEQFSRENGTALSAVDLTWSYASFLTMQRAREGKSGPSWGAASAIAKGVPDKCSGGGVKGAYSTPSATAFPQSEEGTFGTKTSSTSDTGTGTGTVSGTAVSATTSKSAGNRVMPIGFFLH

SEQ ID NO: 18 - protein, 575 aa, Auricularia delicata
  REGION 1..575: note = Auricularia delicata Glucoamylase (mature)
FVAQDARFSSGFGLTLGKRAAVDDYVASEGPIALKGVLDNIGPDGPKSHGAKPGIVVASPSKVDPDYVFAWIRDSALVFKALIDRFVDGRDASRLPLLLQFVSSQSAIQNLDNRSGAARGAGLGEPKFEINQTAFNGDWGRPQRDGPALRATALITLSNYFLSKNNASFVTSTLWPMIQLDLNYVATYWNQPTFDLWEEVNGQSFFTTAVQHRALREGIALAQTLGTTGSVATWSTQASNVLCYLQSYWTPSGAFINSNTNSGRSGIDVNSILTSIHTFSPNTGCDAVTFQPCSDKALAGHLAVVNSFRGSLYPINSGIPAGQAVAIGRYKEDVYYNGNPWYLATFAAAEQLYLALFTWDQQKSIAVTAISQPFFAQFIPNIATGTYASSSSQYATLTSKIREFADGFIAVNQKYTPANGALAEQFTRANGTPISASDLTWSYASALTAFDRRAGIVPGAWPATGLTVAPSCSTAGGGNSTITFKVTAQTVFGENIYLTGSVAALKNWSPENALGPLANPNYPQWQITVTVPASTAIEYKYIRKNGGSVVWESDPNRSIVSPAAGESATVTDTWR

SEQ ID NO: 19 - protein, 617 aa, Talaromyces stipitatus
  REGION 1..617: note = Taloromyces stipitatus glucoamylase (mature)
APGLSPRASTSLDAWLATETTVSLSGILANIGADGAYSKSAKPGVVIASPSTDNPNYYYTWTRDSALTLKVLIDLFRNGNLGLQTVIEEYVNAQAYLQTVSNPSGDLSSGAGLAEPKFNVDMSAFTGSWGRPQRDGPALRAIALIDFGNWLIENGYTSLAANNIWPIVRNDLSYVAQYWSQSGFDLWEEVNSMSFFTVANQHRSLVEGSTFAAKVGASCSWCDSQAPQILCYMQTFWTGSYMNANTGGGRSGKDANTVLTSIATFDPEATCDDVTFQPCSPRALANHKVYTDSFRSVYGLNSGIAEGVAVAVGRYPEDSYYNGNPWFLSNLAAAEQLYDAIYQWNKIGSITITSTSLAFFKDVYSSAAVGTYASGSSAFTSIINAVKTYADGYISVVQSHAMNNGSLSEQFDKNTGAELSARDLTWSYAALLTANMRRNGVVPPSWGAASATSIPSSCTTGSAIGTYSTPTATSWPSTLTSGTGSPGSTTSATGSVSTSVSATTTSAGSCTTPTSVAVTFDEIATTSYGENVYIVGSISQLGSWNTANAIALSASKYTTSNNLWYVTINLPAGTTFQYKYIRKESDGTVKWESDPNRSYTVPSACGVSTATENDTWR

SEQ ID NO: 20 - protein, 576 aa, Piriformospora indica
  REGION 1..576: note = Piriformospora indica glucoamylase (mature)
RASPRENPFNKRVSYTNVADFDTFETPIALAGLYANIGPDGAKSQGAKAGVVIASPSTSDPDYLYTWTRDASLVFKYIVDRFTSGRDSSLRTKIDNFVGHVGRIQQVTNPSGTVSTGGLGEPKFMISEAAFTGEWGRPQRDGPALRATTLIAYANWLRANSNTTHVQNVLWPIISLDLNYVSNNWNSTTFDLWEEVSGASFFTTAAQHRALREGIALATALGAPSSTITAWTTQAQNLLCFLQTYWSPESGFIVANVDPINGAIRSGKDANTVLTTIHTFDPAAGCDSSTFQPCSDKALANLKVYVDSFRSIYPINYGIASNAAVATGRYPEDVYYGGNPWYLTTFAVAEQLYRALQVWDAEGKGIEVTSISLPFFQQFNSSATVTTIPAGSASYTTLTNAIKTFADGFALKGAQFVPSSGALAEQYARNDGTPVSAVDLTWSYASVLTMFDAHDRLAVDSWGAAGLTVPTTCSSGDGKSATVIFKETATTIWGENIYLVGNIEALKNWNTNNPIGPLSTTTYPVWTTLVSIPANTHFEYKFIRKFNGTVTWESGANRWNATGAAGTRLTLNNVWK

SEQ ID NO: 21 - protein, 497 aa, Saccharomycopsis fibuligera
  REGION 1..497: note = Saccharomycopsis fibuligera glucoamylase (mature)
VPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 22 - protein, 785 aa, Saccharomyces cerevisiae
  REGION 1..785: note = S. cerevisiae diastaticus glucoamylase (mature)
FPTALVPRGSSSSNITSSGPSSTPFSSATESFSTGTTVTPSSSKYPGSKTETSVSSTTETTIVPTTTTTSVITPSTTTITTTVCSTGTNSAGETTSGCSPKTITTTVPCSTSPSETASESTTTSPTTPVTTVVSTTVVTTEYSTSTKQGGEITTTFVTKNIPTTYLTTIAPTSSVTTVTNFTPTTITTTVCSTGTNSAGETTSGCSPKTVTTTVPCSTGTGEYTTEATAPVTTAVTTTVVTTESSTGTNSAGKTTTSYTTKSVPTTYVFDFGKGILDQSCGGVFSNNGSSQVQLRDVVLMNGTVVYDSNGAWDSSALEEWLQRQKKVSIERIFENIGPSAVYPSILPGVVIASPSQTHPDYFYQWIRDSALTINSIVSHSADPAIETLLQYLNVSFHLQRTNNTLGAGIGYTNDTVALGDPKWNVDNTAFTEPWGRPQNDGPALRSIAILKIIDYIKQSGTDLGAKYPFQSTADIFDDIVRWDLRFIIDHWNSSGFDLWEEVNGMHFFTLLVQLSAVDRSLSYFNASERSSPFVEELRQTRRDISKFLVDPANGFINGKYNYIVETPMIADTLRSGLDISTLLAANTVHDAPSASHLPFDINDPAVLNTLHHLMLHMRSIYPINDSSKNATGIALGRYPEDVYDGYGVGEGNPWVLATCAASTTLYQLIYRHISEQHDLVVPMNNDCSNAFWSELVFSNLTTLGNDEGYLILEFNTPAFNQTIQKIFQLADSFLVKLKAHVGTDGELSEQFNKYTGFMQGAQHLTWSYTSFWDAYQIRQEVLQSL

SEQ ID NO: 23 - DNA, 57 bp, synthetic construct
  misc_feature 1..57: note = S. cerevisiae MATalpha signal sequence
  source 1..57: mol_type = other DNA
atgagattcccatccatcttcaccgccgtcttgttcgctgcttcttctgctttggct

SEQ ID NO: 24 - protein, 574 aa, Trametes coccinea
  REGION 1..574: note = Glucoamylase from Trametes coccinea (Tcoc_GLA)
MRFPSIFTAVLFAASSALAQSSAADAYVASESPIAKQGVLNNIGPDGSKAHGAKAGIVVASPSTSNPNYLYTWTRDSSLVFKLLIDQFASGEDTSLRGLIDDFTSAEAILQQVPNPSGTVSTGGLGEPKFNIDETAFTDAWGRPQRDGPALRATAIIRYANWLLDNGNTTYVSNTLWPVIQLDLDYVANNWNQSTFDLWEEINSSSFFTTAVQHRALREGATFASRIGQSSVVSGYTTQANNLLCFLQSYWNPSGGYVTANTGGGRSGKDANTVLTSIHTFDPAAGCDAATFQPCSDKALSNLKVYVDAFRSIYTINSGIASNAAVATGRYPEDSYQGGNPWYLTTSAVAEQLYDALYVWDQLGALNVTSTSLAFFQQFSSGISTGTYPASSSTYATLTSAIRTFADGFLAVNAKYTPADGGLAEQYSRNDGTPLSAVDLTWSYAAALTAFAAREGKTYGSWGAAGLTVPASCSGSGGATVAVTFNVQATTVFGENIYITGSVAALQNWSPDNALILSAANYPTWSITVNLPANTVVQYKYIRKFNGQVTWESDPNNQITTPSGGSFTQNDVWR

SEQ ID NO: 25 - DNA, 1725 bp, Trametes coccinea
  misc_feature 1..1725: note = Glucoamylase from Trametes coccinea (Tcoc_GLA.orf)
  source 1..1725: mol_type = genomic DNA
atgagattcccatccatcttcaccgccgtcttgttcgctgcttcttctgctttggctcaatcctctgctgctgatgcttacgttgcttctgaatctccaattgccaagcaaggtgttttgaacaacattggtccagatggttccaaggcccacggtgccaaggctggtattgttgttgcttctccatctacctccaacccaaactacttgtacacctggaccagagactcttctttggttttcaaattgttgattgaccaattcgcttctggtgaagacacttctttgagaggtttgattgatgacttcacttctgctgaagctatcttgcaacaagttccaaacccatccggtaccgtctccactggtgggttgggtgaacctaaattcaacatcgatgaaactgctttcaccgatgcttggggtagacctcaaagagacggtccagctttgagagctaccgccatcatcagatacgctaactggttgttggacaacggtaacaccacctacgtctccaacactttatggccagtcattcaattggatttggactacgttgccaacaactggaaccaatccactttcgacttatgggaagaaatcaactcttcttctttcttcaccactgctgtccaacacagagctttacgtgaaggtgccacctttgcttccagaatcggtcaatcttctgttgtttccggttacaccactcaagctaacaacttgttgtgtttcttgcaatcttactggaacccatctggtggttacgtcactgccaacactggtggtggtcgttctggtaaggatgctaacactgttttgacttctattcacacctttgacccagctgctggttgtgatgctgctactttccaaccatgttctgacaaggctctatccaacttgaaggtctacgtcgacgctttcagatctatctacaccatcaactccggtattgcttccaatgctgctgttgctactggtagatacccagaagactcttaccaaggtggtaacccatggtacttgaccacttctgccgttgctgaacaattatacgatgctttatacgtctgggaccaattgggtgctttgaatgtcacctctacctctttggctttcttccaacaattctcctctggtatttccaccggtacctacccagcttcctcttccacttatgctactttgacctctgccatcagaactttcgctgacggtttcttggctgtcaatgccaagtacactccagctgacggtggtttggccgaacaatactccagaaacgatggtactccattgtctgctgtcgacttgacctggtcttacgccgctgctttgactgcctttgctgccagagaaggtaagacctacggttcctggggtgccgccggtttgaccgtcccagcctcttgttctggttctggtggtgctactgttgctgtcactttcaacgttcaagctaccactgttttcggtgaaaacatctacatcactggttccgttgctgctttacaaaactggtccccagacaacgctttgatcctatccgctgctaactacccaacctggtccatcaccgttaacttgccagctaacaccgttgttcaatacaagtacatcagaaagttcaacggtcaagtcacctgggaatctgacccaaacaaccaaatcaccactccatccggtggttctttcactcaaaacgatgtctggcgataa

SEQ ID NO: 26 - protein, 618 aa, Trichoderma reesei
  REGION 1..618: note = Glucoamylase from Hypocrea jecorina (Hjec_GA)
MRFPSIFTAVLFAASSALASVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 27 - DNA, 1857 bp, Trichoderma reesei
  misc_feature 1..1857: note = Glucoamylase from Hypocrea jecorina (Hjec_GA.orf)
  source 1..1857: mol_type = genomic DNA
atgagattcccatccatcttcaccgccgtcttgttcgctgcttcttctgctttggcttctgtcgatgacttcatctccactgaaactccaattgctttgaacaacttgttgtgtaacgttggtccagacggttgtcgtgccttcggtacttctgccggtgctgtcattgcttctccatctaccatcgacccagactactactacatgtggaccagagactctgctttggttttcaagaacttgatcgaccgtttcaccgaaacttacgatgctggtttgcaaagaagaatcgaacaatacatcactgcccaagtcactctacaaggtttgtccaacccatctggttccttggctgacggttctggtttaggtgaaccaaagttcgaattgaccttgaagccattcaccggtaactggggtagacctcaaagagacggtccagctttgagagccatcgctttgattggttactccaaatggttgattaacaacaactaccaatccactgtctccaatgttatctggccaatcgtcagaaacgatttgaactacgttgctcaatactggaaccaaactggtttcgacttgtgggaagaagttaacggttcctctttcttcaccgttgctaaccaacacagagctttggttgaaggtgctactttggctgccaccttgggtcaatctggttctgcctactcctctgttgctccacaagttctatgtttcttacaaagattctgggtttcttctggtggttacgttgactccaacattaacaccaacgaaggtagaactggtaaggatgtcaactctgtcttgacctccatccacacctttgacccaaacttgggttgtgatgctggtactttccaaccatgttctgacaaggctttatctaacttgaaggtcgttgtcgactctttcagatccatctacggtgttaacaaaggtattccagctggtgctgctgttgccattggtcgttacgctgaagatgtctactacaatggtaacccatggtacttggctacctttgctgctgctgaacaattatacgatgctatctacgtctggaagaagactggttccatcactgtcaccgctacctctttggctttcttccaagaattggtcccaggtgttactgccggtacttactcttcttcctcttccactttcaccaacatcatcaatgccgtctctacctacgctgatgggtttttgtctgaagctgccaagtacgttccagctgatggttctttggccgaacaattcgacagaaactctggtactccattatctgctttgcacttgacctggtcttacgcttctttcttgactgctaccgccagaagagctggtattgttcctccatcctgggctaactcttctgcctctaccattccatccacctgttccggtgcttctgttgttggttcttactccagaccaactgctacctccttcccaccatctcaaaccccaaagcctggtgtcccatccggtaccccatacactccattgccatgtgctactccaacttccgttgccgtcactttccacgaattggtttctactcaattcggtcaaaccgttaaggttgctggtaacgctgctgctttgggtaactggtccacctctgctgccgttgctttggatgccgtcaactatgctgacaaccacccattatggatcggtactgtcaacttggaagctggtgacgtcgttgaatacaaatacattaacgtcggtcaagatggttccgtcacctgggaatccgacccaaaccatacctacactgtcccagctgttgcttgtgttactcaagttgtcaaggaagacacctggcagtcgtaa

SEQ ID NO: 28 - protein, 575 aa, Trametes cingulata
  REGION 1..575: note = Glucoamylase from Trametes cingulata (Tcin_GA)
MRFPSIFTAVLFAASSALAQSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSSTVNLPASTTIEYKYIRKFNGAVTWESDPNNSITTPASGTFTQNDTWR

SEQ ID NO: 29 - DNA, 1728 bp, Trametes cingulata
  misc_feature 1..1728: note = Glucoamylase from Trametes cingulata (Tcin_GA.orf)
  source 1..1728: mol_type = genomic DNA
atgagattcccatccatcttcaccgccgtcttgttcgctgcttcttctgctttggctcaatcttctgctgctgatgcttacgttgcttctgaatctccaattgccaaggctggtgttttggctaacattggtccatctggttccaagtctaacggtgccaaggccggtattgtcattgcttctccttccacttctaacccaaactacttatacacctggaccagagactcttctttggttttcaaggctttgattgaccaattcaccactggtgaagacacctccttgagaactttaattgacgaattcacttccgctgaagctatcttgcaacaagttccaaacccatctggtaccgtctctaccggtggtctaggtgaaccaaagttcaacatcgatgaaactgctttcaccgatgcttggggtagacctcaaagagacggtccagccttgagagctactgccatcatcacctacgccaactggttattggacaacaagaacaccacctacgtcaccaacactttatggccaatcatcaagttggacttggattatgtcgcttccaactggaaccaatccactttcgatttatgggaagaaatcaactcctcttctttcttcactactgctgttcaacacagagctttgagagaaggtgctacctttgctaacagaatcggtcaaacctctgttgtttctggttacaccactcaagccaacaacttgttgtgtttcttgcaatcttactggaacccaactggtggttacatcaccgccaacaccggtggtggtcgttctggtaaggacgctaacactgtcttgacttccattcacactttcgacccagccgctggttgtgatgccgtcactttccaaccatgttccgataaggctttgtctaacttgaaagtttacgtcgatgctttcagatccatctactccatcaactctggtattgcttccaatgctgccgttgctactggtcgttacccagaagactcttacatgggtggtaacccatggtacttgaccacttctgctgttgccgaacaattgtacgatgctttaattgtctggaacaaattgggtgctttgaacgttacctccacttctttgccattcttccaacaattctcttctggtgttaccgtcggtacttacgcttcttcctcttccactttcaagaccttgacttctgccatcaagacttttgctgacggtttcttggctgtcaatgccaagtacactccatccaacggtggtttggctgaacaatactccagatctaacggttctccagtttctgctgtcgacttgacctggtcttacgctgctgctttgacttcctttgctgccagatccggtaagacctacgcttcctggggtgctgctggtttgactgttccaaccacctgttccggttctggtggtgctggtactgttgctgtcactttcaacgtccaagctaccaccgttttcggtgaaaacatctacatcactggttccgttccagctttgcaaaactggtccccagacaatgctttgatcttatctgctgctaactacccaacctggtcctctaccgtcaacttgccagcctctaccaccattgaatacaagtacatcagaaaattcaacggtgctgtcacctgggaatctgacccaaacaactccattaccactccagcttctggtactttcactcaaaacgacacctggaggtaa

SEQ ID NO: 30 - protein, 860 aa, Aspergillus aculeatus
  REGION 1..860: note = beta 1,4-glucosidase (beta-glucosidase) Aacu_BG02
MRFPSIFTAVLFAASSALADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 31 - DNA, 2599 bp, Aspergillus aculeatus
  misc_feature 1..2599: note = beta 1,4-glucosidase (beta-glucosidase) Aacu_BG02
  source 1..2599: mol_type = genomic DNA
ggtctcgaatgagattcccatccatcttcaccgccgtcttgttcgctgcttcttctgctttggctgatgaattggccttctctccaccattctacccttctccatgggctaacggtcaaggtgaatgggctgaagcctaccaaagagctgttgccattgtctcccaaatgactttggatgaaaaggttaacttaaccactggtactggttgggaattggaaaagtgtgttggtcaaactggtggtgtcccaagattgaacatcggtggtatgtgtttgcaagactcccctttgggtatcagagattctgactacaactctgcctttcccgctggtgttaacgtcgctgctacttgggacaagaacttggcttacttgagaggtcaagctatgggtcaagaattctctgacaagggtatcgatgtccaattaggtccagccgctggtccattgggtagatccccagacggtggtagaaactgggaaggtttctctccagacccagctttgaccggtgtcttgtttgctgaaaccatcaaaggtattcaagatgctggtgttgtcgccaccgctaagcactacattttgaacgaacaagaacacttcagacaagttgctgaagctgctggttacggtttcaacatctctgacaccatctcctctaacgttgatgacaagaccattcacgaaatgtacctatggccattcgctgacgctgtcagagccggtgttggtgccatcatgtgttcctacaaccaaatcaacaattcttacggttgtcaaaactcttacactttgaacaagttgttgaaggctgaattaggtttccaaggtttcgtcatgtctgactggggtgcccaccactctggtgtcggttccgctttggctggtttagacatgtccatgccaggtgacatcactttcgactccgctacctctttctggggtaccaacctaaccattgccgtcttaaatggtaccgttcctcaatggagagttgacgatatggctgtcagaatcatggctgcctactacaaggttggtcgtgacagattataccaaccaccaaacttctcctcctggactagagacgaatatggtttcaaatacttctacccacaagaaggtccatacgaaaaggtcaaccacttcgtcaatgttcaaagaaaccattctgaagtcattagaaagttgggtgctgactccaccgtcttgttgaaaaacaacaacgctttgccattgaccggtaaggaaagaaaggttgccattttgggtgaagacgccggttccaactcttacggtgctaatggttgttctgaccgtggttgtgataacggtaccttggctatggcttggggttctggtactgctgaattcccatacttggtcactccagaacaagctatccaagctgaagtcttgaagcacaagggttctgtctacgccatcactgacaactgggctttgtctcaagtcgaaactttggctaagcaagcttccgtttctttggtttttgtcaactccgacgctggtgaaggttacatttccgttgacggtaacgaaggtgacagaaacaacttgactttgtggaagaacggtgacaacttaatcaaggctgctgctaacaactgtaacaacaccattgttgttattcactctgtcggtccagttttggtcgatgaatggtacgatcacccaaacgttaccgccatcttgtgggctggtttgccaggtcaagagtctggtaactctttggctgatgtcttgtacggtagagttaacccaggtgccaaatctccattcacttggggtaagaccagagaagcttacggtgactacttggttagagaattgaacaacggtaacggtgctccacaagacgacttctctgaaggtgttttcatcgattacagaggtttcgacaagagaaacgaaaccccaatctacgaatttggtcacggtctatcttacactactttcaactactccggtttgcacatccaagtcttaaacgcttcttccaacgctcaagttgctaccgaaaccggtgctgctccaaccttcggtcaagtcggtaacgcttccgactacgtctacccagaaggtttgactagaatctccaaattcatctacccatggttgaactccactgatttgaaagcttcttctggtgatccatactacggtgttgacactgctgaacacgttccagaaggtgctactgatggttctccacaaccagttttgccagctggtggtggttctggtggtaacccacgtttgtacgacgaattgatcagagtttccgtcaccgttaagaacaccggtcgtgttgctggtgatgccgttccacaattgtacgtctccttgggtggtccaaacgaaccaaaggttgttttgagaaagttcgatagattgactttgaagccatctgaagaaaccgtctggaccaccactttaactagaagagatttgtccaactgggatgtcgccgctcaagattgggttattacttcttacccaaagaaggttcatgttggttcctcttctcgtcaattgccattacatgctgctttgccaaaggttcaataaaggagacc

SEQ ID NO: 32 - protein, 861 aa, Aspergillus flavus
  REGION 1..861: note = beta 1,4-glucosidase (beta-glucosidase) Asfl_BG11
MRFPSIFTAVLFAASSALAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIRGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAHHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAAENCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPLLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 33 - DNA, 2602 bp, Aspergillus flavus
  misc_feature 1..2602: note = beta 1,4-glucosidase (beta-glucosidase) Asfl_BG11
  source 1..2602: mol_type = genomic DNA
ggtctcgaatgagattcccatccatcttcaccgccgtcttgttcgctgcttcttctgctttggctaaagatgacttggcctactctccaccattttacccatctccatgggctgacggtcaaggagaatgggctgaagtttacaagagagctgttgacattgtctctcaaatgactttgactgaaaaggttaacttgaccaccggtactggttggcaattggaaagatgtgttggtcaaactggttccgtcccaagattaaacatcccatccttgtgtttgcaagattctccattaggtatccgtttctctgactacaactccgccttcccagctggtgttaatgttgctgccacttgggacaagactttggcttacttaagaggtcaagctatgggtgaagaattctccgacaagggtatcgatgtccaattgggtccagccgctggtccattgggtgctcatccagacggtggtagaaactgggaaggtttctccccagacccagctttaaccggtgttttgttcgctgaaaccatcagaggtatccaagacgctggtgtcattgctactgctaagcactacatcatgaacgaacaagaacacttcagacaacaacctgaagccgctgggtacggtttcaacgtctctgattctttatcttccaacgttgacgataagaccatgcacgaattgtacctatggccattcgctgatgccgtcagagccggtgttggtgccgtcatgtgttcctacaaccaaatcaacaactcttacggctgtgaaaactccgaaactttgaacaagttgttgaaggctgaattgggtttccaaggtttcgtcatgtctgactggactgctcaccactctggtgtcggtgctgctttggctggtttggatatgtccatgccaggtgatgtcactttcgactctggtacctctttctggggtgctaacttgactgttggtgttttgaacggtaccattccacaatggagagtcgacgatatggctgttagaattatggctgcttactacaaggttggtagagacaccaagtacactccacctaacttctcctcctggaccagagatgaatacggtttcgctcacaaccacgtttccgaaggtgcctatgaaagagtcaatgaattcgtcgatgttcaaagagatcatgctgatttgattagaagaatcggtgctcaatccaccgttttgttgaagaacaagggtgctctaccattgtccagaaaggaaaagttggttgctttgttaggtgaagatgctggttccaactcctggggtgccaacggttgtgacgacagaggttgtgacaacggtaccttggccatggcttggggttctggtactgctaacttcccttacttggtcactccagaacaagccattcaaaacgaagttttgcaaggtagaggtaatgtcttcgctgttaccgactcctgggctttggacaaaattgctgccgctgctagacaagcctccgtctctttggttttcgttaactctgactctggtgaaggttacttgtctgtcgatggtaacgaaggtgaccgtaacaacatcactttgtggaagaacggtgataatgttgttaagaccgccgctgaaaactgtaacaacaccgtcgtcatcattcactctgttggtccagttttgattgatgaatggtacgaccacccaaacgttaccggtattttgtgggctggtttaccaggtcaagaatctggtaactctattgctgacgttttatacggtcgtgtcaacccaggtgccaaatctccattcacctggggtaagactagagaatcttacggttctccattggttaaggatgctaacaacggtaacggtgctccacaatctgacttcactcaaggtgtcttcatcgattacagacatttcgacaagttcaacgaaaccccaatctacgaattcggttacggtttgtcctacactactttcgaattgtccgacttgcacgtccaaccattaaacgcttccagatacactccaacttctggtatgaccgaagctgccaagaacttcggtgaaatcggtgatgcttctgaatacgtctacccagaaggtttggaacgtatccacgaatttatctacccatggatcaactccactgacttaaaggcttcctctgatgactccaactacggttgggaagactctaagtacatccctgaaggtgctaccgatggttctgctcaaccattgttgccagcttctggtggtgccggtggtaacccaggtttgtacgaagacttgttcagagtttccgtcaaggttaagaacactggtaacgttgctggtgacgaagttccacaattgtacgtttctctaggtggtccaaacgaaccaaaggttgtcttgagaaaattcgaacgtattcacttggctccatctcaagaagctgtctggaccaccactttgaccagaagagacttggctaactgggacgtttctgcccaagactggaccgtcaccccatacccaaagaccatctacgtcggtaactcttctagaaagttgccattgcaagcttctttgccaaaggcacaataaaggagacc

SEQ ID NO: 34 - protein, 860 aa, Aspergillus kawachii
  REGION 1..860: note = beta 1,4-glucosidase (beta-glucosidase) Akaw_BG17
MRFPSIFTAVLFAASSALADELAYSPPYYPSPWANGQGDWAQAYQRAVDIVSQMTLAEKVNLTTGTGWELELCVGQTGGVPRLGVPGMCLQDSPLGVRDSDYNSAFPSGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGYGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTVSVLNGTVPQWRVDDMAVRIMAAYYKVGRDRLWTPPNFSSWTRDEYGYKYYYVSEGPYEKVNHYVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASNYLYPDGLQKITKFIYPWLNSTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKLPLRASLPTVH

SEQ ID NO: 35 - DNA, 2599 bp, Aspergillus kawachii
  misc_feature 1..2599: note = beta 1,4-glucosidase (beta-glucosidase) Akaw_BG17
  source 1..2599: mol_type = genomic DNA
ggtctcgaatgagattcccatccatcttcaccgccgtcttgttcgctgcttcttctgctttggctgatgaattggcttactctccaccttactacccatctccatgggccaacggtcaaggtgactgggctcaagcttaccaaagagctgtcgatatcgtctctcaaatgactttggctgaaaaagtcaacttgaccaccggtactggttgggaattggaactatgtgttggtcaaaccggtggtgttccaagattaggtgtcccaggtatgtgtttgcaagactccccattgggtgtccgtgactctgactacaactccgctttcccttccggtatgaacgttgctgctacttgggacaaaaacttagcctacttgagaggtaaggctatgggtcaagaattctccgacaagggtgctgatatccaattgggtccagctgctggtccattgggtcgttctccagatggtggtagaaactgggaaggtttctctccagacccagctttgtctggtgttttgtttgctgaaaccatcaagggtatccaagatgctggtgttgttgctaccgctaagcactacattgcctacgaacaagaacacttcagacaagctccagaagctcaaggttacggtttcaacatctctgaatctggttctgctaacttggatgacaagaccatgcacgaattatacttatggccattcgctgatgccattagagctggtgccggtgctgttatgtgttcttacaaccaaatcaacaactcttatggttgtcaaaactcttacactttaaacaaattgttgaaggctgaattgggtttccaaggtttcgtcatgtctgactgggccgctcaccacgccggtgtttctggtgctttggccggtctagacatgtccatgccaggtgacgttgattacgactccggtacttcctactggggtaccaatttgactgtttccgtcttgaacggtactgttcctcaatggagagttgatgacatggctgttagaatcatggccgcttactacaaggtcggtcgtgacagattatggaccccaccaaacttctcctcttggaccagagacgaatacggttacaagtactactacgtttctgaaggtccatacgaaaaggtcaaccactacgtcaatgttcaaagaaaccactctgaattgattagaagaattggtgctgactccactgttttgttgaagaacgacggtgctttgccattgactggtaaggaaagattggttgctttgattggtgaagatgccggttccaacccatacggtgccaacggttgttctgaccgtggttgtgataacggtaccttggccatgggttggggttccggtaccgctaacttcccatacttggtcaccccagaacaagccatctccaatgaagtcttgaagaacaagaatggtgttttcaccgctactgacaactgggctatcgaccaaatcgaagctttagctaagactgcttccgtttctttggttttcgttaacgccgactctggtgaaggttacatcaacgtcgatggtaacttgggtgatagaagaaacttgactttgtggagaaatggtgacaacgtcatcaaggccgctgcttctaactgtaacaacactattgtcatcattcactccgttggtcctgttttggtcaacgaatggtacgataacccaaacgtcactgctattttgtggggtggtttgccaggtcaagagtctggtaactccttggctgatgttttgtacggtcgtgtcaacccaggtgccaagtctccattcacctggggtaagactagagaagcctaccaagactacttagtcaccgaaccaaacaacggtaacggtgctccacaagaagacttcgttgaaggtgtcttcattgactacagaggtttcgacaagagaaacgaaaccccaatctacgaattcggctacggtttgtcttacaccactttcaactactccaacttggaagttcaagtcttgtccgctccagcttacgaaccagcttctggtgaaaccgaagccgctccaacctttggtgaagttggtaacgcttccaactacttgtacccagacggtttgcaaaagatcactaagttcatctacccatggttgaactctaccgatctagaagcttcctctggtgatgcttcctacggtcaagattcttctgactacttgccagaaggtgctactgacggttctgctcaaccaattttgccagctggtggtggtccaggtggtaacccaagattgtacgatgaattgatcagagtctctgtcactatcaagaacaccggtaaggttgctggtgacgaagtcccacaattatacgtttccttgggtggtccaaacgaaccaaagattgttttgagacaattcgaaagaattactttgcaaccatctgaagaaactaagtggtccaccactttgaccagaagagatttggctaactggaacgttgaaaagcaagactgggaaatcacttcttacccaaagatggtctttgtcggttcttcctccagaaagttgccattaagagcttctttgccaactgttcattaaaggagacc

SEQ ID NO: 36 - protein, 1035 aa, Talaromyces cellulolyticus
  REGION 1..1035: note = Alpha 1,1-glucosidase (trehalase) Tcel_Tre17
MRFPSIFTAVLFAASSALALPFNERVDQILRSYSPKNLESRSTKHGNSYQTQFSGVTWDQRNWRLQSTVLDQGHYESRGSIANGYIGLNVAGAGPFFELDTAVDGDVINGWPLFSRRQTFAGLAGFYDLQPTTNGSNFPWLSQYGDDSAISGVPHWGGLILDLGDGEYLDATVDNSTISDYTTTYDYKAGVLSWDYKWTPKNSKASFGINYKIFANKLDVNQAVVQLSITPSANGSGSVVNVIDGYSAVRTDFVSSGNESDVIYTAVKPVGVNNVTAWIYAALDGDEAFDFSSAELVNDKPYVHQNDSSIAQSVNVTFTAGTTITINKFVGAASTDAFPDPQSTAREAAMTARRRGFDDLFRSHVSEWAQVMPDDSVDDFTLANGTLPNDTFIIESAVMAVVNPYYLLQNTVGANALRRVNNAPVNDWSIPVGGLTSDSYAGQIFWDADVWMQPGLVAAFPESAKRITNYRTAKYSQALENAKTAYTSSQNQTSFSSDAAIYSWTSGRYGNCTATGPCWDYEYHLNGDIGISLVNQWVVSGDNETFKNTHFPIYNSIATLYGDLLKKNGSYYTLTNMTDPDEYANNVDAGGYTMTLISQTLSNANAFRKQFGMDENTTWTEMADNILLIRENDVTLEYTTMNNSVAVKQADVILSTFPLDYTKNYTTSAALNDLDYYALKQSPDGPGMTYAIFSIVANDVSPSGCSAYTYAQYSYDPYIRGPFFQFSEQLLDDYTANGGTHPAFPFLTGHGGANQVVLYGYLGLRLLPDDMLHIDPNLPPQIPSVKYRTFYWRGWPIQAASNYTHTTIQRATSVAPLSTADPVYANTSISVSVGQNTANSTTYSLPVNGSAIVVPNRQIGSINTVTGNIAQCVSVLSTDAYQPGQYPISAVDGAASTKWQPEFAANVSSLTVDLTSSNASSVSGFYFDWAQAPPTNITVLLHNSSSAALTSSSTHGGSSSVSLNITTSNPYDASSYDANVIALSSSNTTNYTFPAPVAKPRYATLFVQGNQALDETDTKAGNGTGATVAEWAILS

SEQ ID NO: 37 - DNA, 3108 bp, Talaromyces cellulolyticus
  misc_feature 1..3108: note = Alpha 1,1-glucosidase (trehalase) Tcel_Tre17
  source 1..3108: mol_type = genomic DNA
atgagattcccatccatcttcaccgccgtcttgttcgctgcttcttctgctttggctttgccattcaacgaaagagtcgaccaaatcttgagatcttactctcctaagaacttggaatctagatctactaagcacggtaactcttaccaaactcaattctctggtgttacctgggaccaaagaaattggagattacaatctaccgttttggaccaaggtcactacgaatctagaggttctattgctaacggttacattggtttgaacgttgctggtgctggtccctttttcgaattggataccgctgttgatggtgacgttatcaacggttggccattgttctccagaagacaaaccttcgctggtttagccggtttctacgacttgcaaccaaccaccaatggttccaacttcccatggttgtcccaatacggtgatgactctgctatctccggtgtcccacactggggtggtttaattttggatttgggtgatggtgaatacttggacgctaccgtcgataattccaccatctctgactacaccaccacctacgattacaaggccggtgtcttgagctgggactacaagtggaccccaaagaactctaaggcttcttttggtattaactacaaaattttcgccaacaagttggacgtcaaccaagctgtcgtccaattgtccatcactccatctgctaatggttccggttctgttgttaacgttattgacggttactctgctgtcagaactgatttcgtttcctctggtaacgaatctgatgtcatctacactgctgtcaagccagtcggtgtcaacaacgtcactgcttggatctacgctgctttggacggtgacgaagccttcgacttctcttccgctgaattggtcaacgacaagccatatgttcaccaaaacgactcctccattgcccaatctgttaacgtcactttcaccgctggtactactatcaccattaacaagttcgtcggtgctgcttctaccgatgccttcccagacccacaatccactgcccgtgaagctgctatgactgctagaagaagaggtttcgacgatttgttcagatctcacgtttctgaatgggctcaggtcatgccagacgactctgttgatgatttcactttggccaacggtactttgccaaacgacactttcatcattgaatctgccgttatggctgttgtcaacccatactacttgttgcaaaacaccgttggtgccaacgctttaagaagagttaacaacgctcctgttaacgactggtccattccagttggtggtttgacttctgactcttacgctggtcaaatcttctgggacgctgatgtctggatgcaacctggtttagttgctgctttcccagaatctgccaagagaattactaactaccgtactgctaagtactctcaagctttggaaaacgccaagactgcttacacttcctctcaaaaccaaacctctttctcttctgacgctgctatctactcttggacctccggtagatacggtaactgtaccgctaccggtccatgttgggactacgaataccacttgaacggtgatatcggtatctctttagttaaccaatgggttgtctccggtgacaatgaaactttcaagaacacccacttcccaatctacaactccattgctaccttgtacggtgatttgctaaagaagaacggttcttattacactttgactaacatgactgatccagacgaatacgctaacaatgtcgacgctggtggttacaccatgactttgatctctcaaaccttgtctaacgctaacgccttcagaaagcaattcggtatggacgaaaacaccacctggactgaaatggctgacaacatcttgttgatcagagaaaacgatgtcactttggaatacactaccatgaacaactctgttgctgttaagcaagctgacgttattttgtccacttttcctttggattacactaaaaactacactacctctgctgctttgaacgacttggactactacgctttgaagcaatctccagacggtccaggtatgacctacgctatcttctccatcgtcgccaacgacgtttctccatccggttgttctgcctatacttacgctcaatactcttacgacccatacatcagaggtccattcttccaattctccgaacaattattggatgattacactgccaacggtggtacccacccagccttcccattcttaaccggtcacggtggtgctaaccaagttgtcttatacggttacttgggtttgcgtttgttgccagatgacatgttgcacattgatccaaacctaccaccacaaattccatctgtcaagtacagaactttctactggagaggttggccaatccaagctgccagcaactacacccacaccactatccaaagagctacctccgttgctccattatccactgctgacccagtttacgccaacacctctatttccgtctctgttggtcaaaacactgctaactccactacttactctttgccagttaacggttctgctatcgttgttccaaacagacaaatcggttctattaacactgtcaccggtaacattgctcaatgtgtctctgtcttgtccaccgatgcttaccaaccaggtcaatacccaatctctgctgttgacggtgccgcttccactaagtggcaaccagaatttgctgctaatgtctcttctttgaccgttgacttgacctcttccaacgcatcctctgtttctggtttctacttcgattgggctcaagctccaccaactaacatcaccgtcttgttgcacaactcttcctccgccgctttgacatcttcctctacccatggtggttcctcctctgtctccttgaacatcaccacctccaacccttacgacgcttcctcctacgatgccaacgttatcgctttgtcctcctctaacaccactaactacactttcccagctccagtcgctaagccaagatacgctactttgttcgttcaaggtaaccaagccttggatgaaactgataccaaggctggtaacggtaccggtgctaccgttgccgaatgggctattctatcttaa

SEQ ID NO: 38 - DNA, 50 bp, synthetic construct
  misc_feature 1..50: note = Connector 2J
  source 1..50: mol_type = other DNA
tcctgtacgcactttgtcccacaaattcccgattccgcaatttgttcgcc

SEQ ID NO: 39 - DNA, 50 bp, synthetic construct
  misc_feature 1..50: note = Connector 2K
  source 1..50: mol_type = other DNA
aaaagtggagctagtctatttctatttacatacctctcatttctcatttc

SEQ ID NO: 40 - DNA, 50 bp, synthetic construct
  misc_feature 1..50: note = Connector 2L
  source 1..50: mol_type = other DNA
cgagaacgtcggcggttaaaatatattaccctgaacgtggtgaattgaag

SEQ ID NO: 41 - DNA, 50 bp, synthetic construct
  misc_feature 1..50: note = Connector 2M
  source 1..50: mol_type = other DNA
catttttcattttatgttaaaacaatttcaggtttaccttttattctgct

SEQ ID NO: 42 - DNA, 50 bp, synthetic construct
  misc_feature 1..50: note = Connector 2N
  source 1..50: mol_type = other DNA
tccgctctaaccgaaaaggaaggagttagacaacctgaagtctaggtccc

SEQ ID NO: 43 - DNA, 2620 bp, synthetic construct
  misc_feature 1..2620: note = Fragment A: 2J-Spar_TDH3.pro-Pstr_GA.orf_0009-Sc_ADH1.ter-2K
  source 1..2620: mol_type = other DNA
tcctgtacgcactttgtcccacaaattcccgattccgcaatttgttcgccgtgcgttttatttctgctgccatccgtaaatgccaggatttgagcgggttacacaatatatctcatattttcggtgtctgggtcattactttactcttggcatccactaaatatattggatcctgctttttaaactggcttccagaaaaaaatcaatggagtgatgcaaactgcctggagtaaaagatgacacaaggcgattgacctacgcatgtatctatctcattttcttacaccttctatttcattctaactctttgatttggaaaacacctaagaaaaaaaaggttgaaatcagttccctgaaattgtccccctacttgactaataaatatataaagacggtaggtattgactgtaattcgtaaatctatacttcttaaacttcttcaaatttacttttttggatagtcttatttttggtttcaataccccaagaacttagtttcaaataaatacacatacaaacaaaatgagattcccatccatcttcaccgccgtcttgttcgctgcttcttctgctttggcttcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaaatcaactcctcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagatgttttcatgggtggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacctcgaacaggatatttcgatcttggatacgtactcgcttgtgtctggtttcgt

SEQ ID NO: 44 - DNA, 2422 bp, synthetic construct
  misc_feature 1..2422: note = Fragment B: 2K-Sc_PFY1.pro-Tcoc_GLA.orf-Sc_TDH1.ter-2L
  source 1..2422: mol_type = other DNA
gaacaggatatttcgatcttggatacgtactcgcttgtgtctggtttcgtgtgcaggagacgttactttgtttatatatattagtatgtacaatcgcaaagaaatggagtgatgacatgttgtagtatttagtatgaggttactgtgtgggaggttttaccatgatttttggcgagaacacgccatgaaatgtctttgtacgaaactcattacccgcattaatattttttttctttttaaagctcagttgaccctttctcattcccttcttaaaacaactgtgtgatccttgagaaaagataaattacatacacaacataaacccaactacgatcgcaaataatgagattcccatccatcttcaccgccgtcttgttcgctgcttcttctgctttggctcaatcctctgctgctgatgcttacgttgcttctgaatctccaattgccaagcaaggtgttttgaacaacattggtccagatggttccaaggcccacggtgccaaggctggtattgttgttgcttctccatctacctccaacccaaactacttgtacacctggaccagagactcttctttggttttcaaattgttgattgaccaattcgcttctggtgaagacacttctttgagaggtttgattgatgacttcacttctgctgaagctatcttgcaacaagttccaaacccatccggtaccgtctccactggtgggttgggtgaacctaaattcaacatcgatgaaactgctttcaccgatgcttggggtagacctcaaagagacggtccagctttgagagctaccgccatcatcagatacgctaactggttgttggacaacggtaacaccacctacgtctccaacactttatggccagtcattcaattggatttggactacgttgccaacaactggaaccaatccactttcgacttatgggaagaaatcaactcttcttctttcttcaccactgctgtccaacacagagctttacgtgaaggtgccacctttgcttccagaatcggtcaatcttctgttgtttccggttacaccactcaagctaacaacttgttgtgtttcttgcaatcttactggaacccatctggtggttacgtcactgccaacactggtggtggtcgttctggtaaggatgctaacactgttttgacttctattcacacctttgacccagctgctggttgtgatgctgctactttccaaccatgttctgacaaggctctatccaacttgaaggtctacgtcgacgctttcagatctatctacaccatcaactccggtattgcttccaatgctgctgttgctactggtagatacccagaagactcttaccaaggtggtaacccatggtacttgaccacttctgccgttgctgaacaattatacgatgctttatacgtctgggaccaattgggtgctttgaatgtcacctctacctctttggctttcttccaacaattctcctctggtatttccaccggtacctacccagcttcctcttccacttatgctactttgacctctgccatcagaactttcgctgacggtttcttggctgtcaatgccaagtacactccagctgacggtggtttggccgaacaatactccagaaacgatggtactccattgtctgctgtcgacttgacctggtcttacgccgctgctttgactgcctttgctgccagagaaggtaagacctacggttcctggggtgccgccggtttgaccgtcccagcctcttgttctggttctggtggtgctactgttgctgtcactttcaacgttcaagctaccactgttttcggtgaaaacatctacatcactggttccgttgctgctttacaaaactggtccccagacaacgctttgatcctatccgctgctaactacccaacctggtccatcaccgttaacttgccagctaacaccgttgttcaatacaagtacatcagaaagttcaacggtcaagtcacctgggaatctgacccaaacaaccaaatcaccactccatccggtggttctttcactcaaaacgatgtctggcgataaaataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgccttcctctctgcaccttctttcgcttgcactgttcccagactccaagatttgtatac

SEQ ID NO: 45 - DNA, 3592 bp, synthetic construct
  misc_feature 1..3592: note = Fragment C: 2L-Sc_ACT1.pro-Akaw_BG17.orf-Sc_ENO1.ter-2M
  source 1..3592: mol_type = other DNA
tctgcaccttctttcgcttgcactgttcccagactccaagatttgtatacgtgcaacatatatacacaattacagtaacaataacaagaggacagatactaccaaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttctttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaacaatgagattcccatccatcttcaccgccgtcttgttcgctgcttcttctgctttggctgatgaattggcttactctccaccttactacccatctccatgggccaacggtcaaggtgactgggctcaagcttaccaaagagctgtcgatatcgtctctcaaatgactttggctgaaaaagtcaacttgaccaccggtactggttgggaattggaactatgtgttggtcaaaccggtggtgttccaagattaggtgtcccaggtatgtgtttgcaagactccccattgggtgtccgtgactctgactacaactccgctttcccttccggtatgaacgttgctgctacttgggacaaaaacttagcctacttgagaggtaaggctatgggtcaagaattctccgacaagggtgctgatatccaattgggtccagctgctggtccattgggtcgttctccagatggtggtagaaactgggaaggtttctctccagacccagctttgtctggtgttttgtttgctgaaaccatcaagggtatccaagatgctggtgttgttgctaccgctaagcactacattgcctacgaacaagaacacttcagacaagctccagaagctcaaggttacggtttcaacatctctgaatctggttctgctaacttggatgacaagaccatgcacgaattatacttatggccattcgctgatgccattagagctggtgccggtgctgttatgtgttcttacaaccaaatcaacaactcttatggttgtcaaaactcttacactttaaacaaattgttgaaggctgaattgggtttccaaggtttcgtcatgtctgactgggccgctcaccacgccggtgtttctggtgctttggccggtctagacatgtccatgccaggtgacgttgattacgactccggtacttcctactggggtaccaatttgactgtttccgtcttgaacggtactgttcctcaatggagagttgatgacatggctgttagaatcatggccgcttactacaaggtcggtcgtgacagattatggaccccaccaaacttctcctcttggaccagagacgaatacggttacaagtactactacgtttctgaaggtccatacgaaaaggtcaaccactacgtcaatgttcaaagaaaccactctgaattgattagaagaattggtgctgactccactgttttgttgaagaacgacggtgctttgccattgactggtaaggaaagattggttgctttgattggtgaagatgccggttccaacccatacggtgccaacggttgttctgaccgtggttgtgataacggtaccttggccatgggttggggttccggtaccgctaacttcccatacttggtcaccccagaacaagccatctccaatgaagtcttgaagaacaagaatggtgttttcaccgctactgacaactgggctatcgaccaaatcgaagctttagctaagactgcttccgtttctttggttttcgttaacgccgactctggtgaaggttacatcaacgtcgatggtaacttgggtgatagaagaaacttgactttgtggagaaatggtgacaacgtcatcaaggccgctgcttctaactgtaacaacactattgtcatcattcactccgttggtcctgttttggtcaacgaatggtacgataacccaaacgtcactgctattttgtggggtggtttgccaggtcaagagtctggtaactccttggctgatgttttgtacggtcgtgtcaacccaggtgccaagtctccattcacctggggtaagactagagaagcctaccaagactacttagtcaccgaaccaaacaacggtaacggtgctccacaagaagacttcgttgaaggtgtcttcattgactacagaggtttcgacaagagaaacgaaaccccaatctacgaattcggctacggtttgtcttacaccactttcaactactccaacttggaagttcaagtcttgtccgctccagcttacgaaccagcttctggtgaaaccgaagccgctccaacctttggtgaagttggtaacgcttccaactacttgtacccagacggtttgcaaaagatcactaagttcatctacccatggttgaactctaccgatctagaagcttcctctggtgatgcttcctacggtcaagattcttctgactacttgccagaaggtgctactgacggttctgctcaaccaattttgccagctggtggtggtccaggtggtaacccaagattgtacgatgaattgatcagagtctctgtcactatcaagaacaccggtaaggttgctggtgacgaagtcccacaattatacgtttccttgggtggtccaaacgaaccaaagattgttttgagacaattcgaaagaattactttgcaaccatctgaagaaactaagtggtccaccactttgaccagaagagatttggctaactggaacgttgaaaagcaagactgggaaatcacttcttacccaaagatggtctttgtcggttcttcctccagaaagttgccattaagagcttctttgccaactgttcattaaaagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcccctcccaatacaaggcagaactttcctccaccgtatccgctcgcaatttctttc

SEQ ID NO: 46 - DNA, 3803 bp, synthetic construct
  misc_feature 1..3803: note = Fragment D: 2M-Sc_YKT6.pro-Tcel_Tre17.orf-Sc_CYC1.ter-2N
  source 1..3803: mol_type = other DNA
ccaatacaaggcagaactttcctccaccgtatccgctcgcaatttctttcgtgctttactgataatatacactctttggatcgagcccacttccagttggtaattggtgttccacaatttcagcattacatgtttttaaaccaaaattcggctccttttccctttttttcttattgggtggcgtgccgtacagaacgattggcttggtgtgaaatcaagagcaagcacaatagatatcaacatgaacaatatacaaaagtctctggcacagtttgactgcgttagaccaggctagggcatttctgaagctttacgtatcactagagaagttattttggcaatgagattcccatccatcttcaccgccgtcttgttcgctgcttcttctgctttggctttgccattcaacgaaagagtcgaccaaatcttgagatcttactctcctaagaacttggaatctagatctactaagcacggtaactcttaccaaactcaattctctggtgttacctgggaccaaagaaattggagattacaatctaccgttttggaccaaggtcactacgaatctagaggttctattgctaacggttacattggtttgaacgttgctggtgctggtccctttttcgaattggataccgctgttgatggtgacgttatcaacggttggccattgttctccagaagacaaaccttcgctggtttagccggtttctacgacttgcaaccaaccaccaatggttccaacttcccatggttgtcccaatacggtgatgactctgctatctccggtgtcccacactggggtggtttaattttggatttgggtgatggtgaatacttggacgctaccgtcgataattccaccatctctgactacaccaccacctacgattacaaggccggtgtcttgagctgggactacaagtggaccccaaagaactctaaggcttcttttggtattaactacaaaattttcgccaacaagttggacgtcaaccaagctgtcgtccaattgtccatcactccatctgctaatggttccggttctgttgttaacgttattgacggttactctgctgtcagaactgatttcgtttcctctggtaacgaatctgatgtcatctacactgctgtcaagccagtcggtgtcaacaacgtcactgcttggatctacgctgctttggacggtgacgaagccttcgacttctcttccgctgaattggtcaacgacaagccatatgttcaccaaaacgactcctccattgcccaatctgttaacgtcactttcaccgctggtactactatcaccattaacaagttcgtcggtgctgcttctaccgatgccttcccagacccacaatccactgcccgtgaagctgctatgactgctagaagaagaggtttcgacgatttgttcagatctcacgtttctgaatgggctcaggtcatgccagacgactctgttgatgatttcactttggccaacggtactttgccaaacgacactttcatcattgaatctgccgttatggctgttgtcaacccatactacttgttgcaaaacaccgttggtgccaacgctttaagaagagttaacaacgctcctgttaacgactggtccattccagttggtggtttgacttctgactcttacgctggtcaaatcttctgggacgctgatgtctggatgcaacctggtttagttgctgctttcccagaatctgccaagagaattactaactaccgtactgctaagtactctcaagctttggaaaacgccaagactgcttacacttcctctcaaaaccaaacctctttctcttctgacgctgctatctactcttggacctccggtagatacggtaactgtaccgctaccggtccatgttgggactacgaataccacttgaacggtgatatcggtatctctttagttaaccaatgggttgtctccggtgacaatgaaactttcaagaacacccacttcccaatctacaactccattgctaccttgtacggtgatttgctaaagaagaacggttcttattacactttgactaacatgactgatccagacgaatacgctaacaatgtcgacgctggtggttacaccatgactttgatctctcaaaccttgtctaacgctaacgccttcagaaagcaattcggtatggacgaaaacaccacctggactgaaatggctgacaacatcttgttgatcagagaaaacgatgtcactttggaatacactaccatgaacaactctgttgctgttaagcaagctgacgttattttgtccacttttcctttggattacactaaaaactacactacctctgctgctttgaacgacttggactactacgctttgaagcaatctccagacggtccaggtatgacctacgctatcttctccatcgtcgccaacgacgtttctccatccggttgttctgcctatacttacgctcaatactcttacgacccatacatcagaggtccattcttccaattctccgaacaattattggatgattacactgccaacggtggtacccacccagccttcccattcttaaccggtcacggtggtgctaaccaagttgtcttatacggttacttgggtttgcgtttgttgccagatgacatgttgcacattgatccaaacctaccaccacaaattccatctgtcaagtacagaactttctactggagaggttggccaatccaagctgccagcaactacacccacaccactatccaaagagctacctccgttgctccattatccactgctgacccagtttacgccaacacctctatttccgtctctgttggtcaaaacactgctaactccactacttactctttgccagttaacggttctgctatcgttgttccaaacagacaaatcggttctattaacactgtcaccggtaacattgctcaatgtgtctctgtcttgtccaccgatgcttaccaaccaggtcaatacccaatctctgctgttgacggtgccgcttccactaagtggcaaccagaatttgctgctaatgtctcttctttgaccgttgacttgacctcttccaacgcatcctctgtttctggtttctacttcgattgggctcaagctccaccaactaacatcaccgtcttgttgcacaactcttcctccgccgctttgacatcttcctctacccatggtggttcctcctctgtctccttgaacatcaccacctccaacccttacgacgcttcctcctacgatgccaacgttatcgctttgtcctcctctaacaccactaactacactttcccagctccagtcgctaagccaagatacgctactttgttcgttcaaggtaaccaagccttggatgaaactgataccaaggctggtaacggtaccggtgctaccgttgccgaatgggctattctatcttaaaacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagcttcgcagtttacactctcatcgtcctcggtgttgcacacgaatacgacacttcccaatacactttgcctctccaagg

SEQ ID NO: 47 - DNA, 2752 bp, synthetic construct
  misc_feature 1..2752: note = Fragment E: 2J-Sc_PGK1.pro-Pstr_GA.orf_0009-Sc_ENO1.ter-2K
  source 1..2752: mol_type = other DNA
tcctgtacgcactttgtcccacaaattcccgattccgcaatttgttcgccgtgcgggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgagattcccatccatcttcaccgccgtcttgttcgctgcttcttctgctttggcttcttccaagtctgtttctgcttacatctcctctgaatctccaattgctcactccaagttgttggacaacattggtccagatggtgccaaggctccaggtgctttcccaggtgtcgttgttgcttctccatctactgacaacccaaactactactactcctggattagagactcttctttggttttcaagactttaatcgatgactacgttaacggtaagaacacttccaagtctttgagatctttgattgatgacttcgtcactgcttcttccgttttccaacaaaccccaaacccatctggtaacgtttccaccggtggtttgggtgaaccaaagttctacgtcaacgaaactgccttcttggactcttggggtagacctcaaagagatggtccagctttgagatccactgctttgatcacttatgccaactacttgttggacaacgacaacaccacctgggttaaggacactttatggccaatcatcgaattggatgtcaactacgtttctgatttctggaactacaccactttcgacttatgggaagaaatcaactcctcttctttcttcaccactgccgtccaacacagagctttgagacaagcttccaaattggccaagactttggacaagactgacaacattgactcctggaacactcaagctgacaacgtcttgtgtttcttgcaatcttactggaacggttctgccatcattgctaacaccggtggtggtcgttctggtattgatgctaacaccgtcttggcttccatccacacctttgactcttctgccggttgtgatgctaccactttccaaccatgttctgacaaggctttgtccaacttgaaggtttacgtcgatgctttcagatccatctacgaaatcaactctggtattgacccaaatgctgctgttgctactggtcgttacccagaagatgttttcatgggtggtaacccatggtacttggccactgctgctgttgctgaacaattatacgatgctttgtacgtctggaacaccaccggttccttggaaatcactgacatctctttgcctttcttccaacaattcgactctgatgtcaagactggtacctactctgatgacgacacctttgactctttgatctcctccatccaatcctttgctgacggtttcttggaaattcacgctaaatacactccagatgacggtgctttgtctgaagaattctccaagaccgatggttctcaaacctccgctgctgacttgacctggtcctacgccgctgctttgactgctttcgatgccagatccagagatgctgctgtcaaatggggtgccaagggtttacaagtcccagatggtacttgtaagaccaatgaaggtggtgacgacggtctaggtgtcccagttactttcttggttaaggatgctgaaaccgttgaaggtcaatctgtctacatcactggttccattgctactttgaaatcctggtccccagatgacgctttgttgatgtctccatctgactacccaacctggactttgaccgttaacttgtctgcctccgaatccgttcaatacaagtacatcaagaaggacactgctggtaccgttatctgggaatctgacccaaacaactctctattagtcccatctggtggttctgtcaccaccgatgacacctggagataaaagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcccctcgaacaggatatttcgatcttggatacgtactcgcttgtgtctggtttcgt

SEQ ID NO: 48 - DNA, 2683 bp, synthetic construct
  misc_feature 1..2683: note = Fragment F: 2K-Sc_RPS3.pro-Tcoc_GLA.orf-Sc_TDH1.ter-2L
  source 1..2683: mol_type = other DNA
gaacaggatatttcgatcttggatacgtactcgcttgtgtctggtttcgtgtgcaatccaagtaaaaggatggatatcgttatactaaaagcaacacagaaaaggtccacgtcagttccacacaataacatttacgtagtgttcacgcgaagcagttacatctcaactaacataattgctggtgagcctacaacactgcatgcgtaaacgtcaacgggattacgttagtatttttggccgccggtaaattctcttgtttttttttcttgatttcacttcttttcatgttcctttggaataatctaattcctcatgattaaatgagactgttttttgtttccgtaacatccatacctttcctgtataatattcttgctgtaaagtttgttttttttatgaaaaaaacattttcttttcttgagatgaggcgccgcgagcctttctcccatgggcagtggtaaattttccaaatcaatgcagctctttgaaatacaacagcatttttcatacattttaagcaatttctagtttgtagatattgttagattagtttttgaacattgttttgataactgaaaataaaacagcaaacaaactacaaaaatgagattcccatccatcttcaccgccgtcttgttcgctgcttcttctgctttggctcaatcctctgctgctgatgcttacgttgcttctgaatctccaattgccaagcaaggtgttttgaacaacattggtccagatggttccaaggcccacggtgccaaggctggtattgttgttgcttctccatctacctccaacccaaactacttgtacacctggaccagagactcttctttggttttcaaattgttgattgaccaattcgcttctggtgaagacacttctttgagaggtttgattgatgacttcacttctgctgaagctatcttgcaacaagttccaaacccatccggtaccgtctccactggtgggttgggtgaacctaaattcaacatcgatgaaactgctttcaccgatgcttggggtagacctcaaagagacggtccagctttgagagctaccgccatcatcagatacgctaactggttgttggacaacggtaacaccacctacgtctccaacactttatggccagtcattcaattggatttggactacgttgccaacaactggaaccaatccactttcgacttatgggaagaaatcaactcttcttctttcttcaccactgctgtccaacacagagctttacgtgaaggtgccacctttgcttccagaatcggtcaatcttctgttgtttccggttacaccactcaagctaacaacttgttgtgtttcttgcaatcttactggaacccatctggtggttacgtcactgccaacactggtggtggtcgttctggtaaggatgctaacactgttttgacttctattcacacctttgacccagctgctggttgtgatgctgctactttccaaccatgttctgacaaggctctatccaacttgaaggtctacgtcgacgctttcagatctatctacaccatcaactccggtattgcttccaatgctgctgttgctactggtagatacccagaagactcttaccaaggtggtaacccatggtacttgaccacttctgccgttgctgaacaattatacgatgctttatacgtctgggaccaattgggtgctttgaatgtcacctctacctctttggctttcttccaacaattctcctctggtatttccaccggtacctacccagcttcctcttccacttatgctactttgacctctgccatcagaactttcgctgacggtttcttggctgtcaatgccaagtacactccagctgacggtggtttggccgaacaatactccagaaacgatggtactccattgtctgctgtcgacttgacctggtcttacgccgctgctttgactgcctttgctgccagagaaggtaagacctacggttcctggggtgccgccggtttgaccgtcccagcctcttgttctggttctggtggtgctactgttgctgtcactttcaacgttcaagctaccactgttttcggtgaaaacatctacatcactggttccgttgctgctttacaaaactggtccccagacaacgctttgatcctatccgctgctaactacccaacctggtccatcaccgttaacttgccagctaacaccgttgttcaatacaagtacatcagaaagttcaacggtcaagtcacctgggaatctgacccaaacaaccaaatcaccactccatccggtggttctttcactcaaaacgatgtctggcgataaaataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgccttcctctctgcaccttctttcgcttgcactgttcccagactccaagatttgtatac

SEQ ID NO: 49 - DNA, 3543 bp, synthetic construct
  misc_feature 1..3543: note = Fragment G: 2L-Sc_ZUO1.pro-Akaw_BG17.orf-Sc_ADH1.ter-2M
  source 1..3543: mol_type = other DNA
tctgcaccttctttcgcttgcactgttcccagactccaagatttgtatacgtgcctggcgttttatcttttatgcatccaatatctaatattacttccgatcacgcatttagttctgattacagcagaaatcgtagcgcgatgagacatttcatcaaatggccttttttttttgggcaatttttttatatcttgaaatgatagttgccttgtactttcaaccgttcatttcattaagaacttgactaaatatgaacatttcttaaaaaaaaggttgacatataaaaataatcgaatataaacgatggaatttttataaaattaaacacatatatatatatatattaactataaatatgtcaaagaaaccatacaatcatagatttataactatcttttggatgacattaatgaacataacgctcctaatacaaatgtccaaaaaatattacccgcaaatacgaatcttttttttttctcgatgaaattttgcaaagagttcgaaatttttatttcaagagctggtagagaaaatttcataaggttttcctaccgatgcttttataaaatcttcgttttgtctcacatataccaacaagagtaacaatgagattcccatccatcttcaccgccgtcttgttcgctgcttcttctgctttggctgatgaattggcttactctccaccttactacccatctccatgggccaacggtcaaggtgactgggctcaagcttaccaaagagctgtcgatatcgtctctcaaatgactttggctgaaaaagtcaacttgaccaccggtactggttgggaattggaactatgtgttggtcaaaccggtggtgttccaagattaggtgtcccaggtatgtgtttgcaagactccccattgggtgtccgtgactctgactacaactccgctttcccttccggtatgaacgttgctgctacttgggacaaaaacttagcctacttgagaggtaaggctatgggtcaagaattctccgacaagggtgctgatatccaattgggtccagctgctggtccattgggtcgttctccagatggtggtagaaactgggaaggtttctctccagacccagctttgtctggtgttttgtttgctgaaaccatcaagggtatccaagatgctggtgttgttgctaccgctaagcactacattgcctacgaacaagaacacttcagacaagctccagaagctcaaggttacggtttcaacatctctgaatctggttctgctaacttggatgacaagaccatgcacgaattatacttatggccattcgctgatgccattagagctggtgccggtgctgttatgtgttcttacaaccaaatcaacaactcttatggttgtcaaaactcttacactttaaacaaattgttgaaggctgaattgggtttccaaggtttcgtcatgtctgactgggccgctcaccacgccggtgtttctggtgctttggccggtctagacatgtccatgccaggtgacgttgattacgactccggtacttcctactggggtaccaatttgactgtttccgtcttgaacggtactgttcctcaatggagagttgatgacatggctgttagaatcatggccgcttactacaaggtcggtcgtgacagattatggaccccaccaaacttctcctcttggaccagagacgaatacggttacaagtactactacgtttctgaaggtccatacgaaaaggtcaaccactacgtcaatgttcaaagaaaccactctgaattgattagaagaattggtgctgactccactgttttgttgaagaacgacggtgctttgccattgactggtaaggaaagattggttgctttgattggtgaagatgccggttccaacccatacggtgccaacggttgttctgaccgtggttgtgataacggtaccttggccatgggttggggttccggtaccgctaacttcccatacttggtcaccccagaacaagccatctccaatgaagtcttgaagaacaagaatggtgttttcaccgctactgacaactgggctatcgaccaaatcgaagctttagctaagactgcttccgtttctttggttttcgttaacgccgactctggtgaaggttacatcaacgtcgatggtaacttgggtgatagaagaaacttgactttgtggagaaatggtgacaacgtcatcaaggccgctgcttctaactgtaacaacactattgtcatcattcactccgttggtcctgttttggtcaacgaatggtacgataacccaaacgtcactgctattttgtggggtggtttgccaggtcaagagtctggtaactccttggctgatgttttgtacggtcgtgtcaacccaggtgccaagtctccattcacctggggtaagactagagaagcctaccaagactacttagtcaccgaaccaaacaacggtaacggtgctccacaagaagacttcgttgaaggtgtcttcattgactacagaggtttcgacaagagaaacgaaaccccaatctacgaattcggctacggtttgtcttacaccactttcaactactccaacttggaagttcaagtcttgtccgctccagcttacgaaccagcttctggtgaaaccgaagccgctccaacctttggtgaagttggtaacgcttccaactacttgtacccagacggtttgcaaaagatcactaagttcatctacccatggttgaactctaccgatctagaagcttcctctggtgatgcttcctacggtcaagattcttctgactacttgccagaaggtgctactgacggttctgctcaaccaattttgccagctggtggtggtccaggtggtaacccaagattgtacgatgaattgatcagagtctctgtcactatcaagaacaccggtaaggttgctggtgacgaagtcccacaattatacgtttccttgggtggtccaaacgaaccaaagattgttttgagacaattcgaaagaattactttgcaaccatctgaagaaactaagtggtccaccactttgaccagaagagatttggctaactggaacgttgaaaagcaagactgggaaatcacttcttacccaaagatggtctttgtcggttcttcctccagaaagttgccattaagagcttctttgccaactgttcattaaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacctcccaatacaaggcagaactttcctccaccgtatccgctcgcaatttctttc

SEQ ID NO: 50 - DNA, 4120 bp, synthetic construct
  misc_feature 1..4120: note = Fragment H: 2M-Sc_MYO4.pro-Tcel_Tre17.orf-Sc_AQR1.ter-2N
  source 1..4120: mol_type = other DNA
ccaatacaaggcagaactttcctccaccgtatccgctcgcaatttctttcgtgcataacaatagaacaatagctttttgaccttccccttattttatttcaaaaggtaacagttagggctattcttaaactttctttagggtacacaatgaaaacatcaagattgagttaaaacccttaaaataaaactaggtctttaagaaactcactctccgggttatccatagaatgtttaccattctttagggttctgaactaatgcgaaaaaaaaaaaaggaaaagactgaaaaattcgaaaatatctcgaagtttagcagtagtagatgagaatagggtgtcttttatcgaaaacctgcaattgtaatacaacctttttttgaaagtcagcctttattctgattctgcatactcaattcctcaattcctacggagttatcacctttttttttaccttctcttcttttcttattgttctagctgaaaacattgttaccagtttggcgagacaatttattttcaatacgatacccttttgttcttctttttataattcaatctaattctaaaacacaaaaaaacaaaaaaaatcctataaccagttctcccgaatgagattcccatccatcttcaccgccgtcttgttcgctgcttcttctgctttggctttgccattcaacgaaagagtcgaccaaatcttgagatcttactctcctaagaacttggaatctagatctactaagcacggtaactcttaccaaactcaattctctggtgttacctgggaccaaagaaattggagattacaatctaccgttttggaccaaggtcactacgaatctagaggttctattgctaacggttacattggtttgaacgttgctggtgctggtccctttttcgaattggataccgctgttgatggtgacgttatcaacggttggccattgttctccagaagacaaaccttcgctggtttagccggtttctacgacttgcaaccaaccaccaatggttccaacttcccatggttgtcccaatacggtgatgactctgctatctccggtgtcccacactggggtggtttaattttggatttgggtgatggtgaatacttggacgctaccgtcgataattccaccatctctgactacaccaccacctacgattacaaggccggtgtcttgagctgggactacaagtggaccccaaagaactctaaggcttcttttggtattaactacaaaattttcgccaacaagttggacgtcaaccaagctgtcgtccaattgtccatcactccatctgctaatggttccggttctgttgttaacgttattgacggttactctgctgtcagaactgatttcgtttcctctggtaacgaatctgatgtcatctacactgctgtcaagccagtcggtgtcaacaacgtcactgcttggatctacgctgctttggacggtgacgaagccttcgacttctcttccgctgaattggtcaacgacaagccatatgttcaccaaaacgactcctccattgcccaatctgttaacgtcactttcaccgctggtactactatcaccattaacaagttcgtcggtgctgcttctaccgatgccttcccagacccacaatccactgcccgtgaagctgctatgactgctagaagaagaggtttcgacgatttgttcagatctcacgtttctgaatgggctcaggtcatgccagacgactctgttgatgatttcactttggccaacggtactttgccaaacgacactttcatcattgaatctgccgttatggctgttgtcaacccatactacttgttgcaaaacaccgttggtgccaacgctttaagaagagttaacaacgctcctgttaacgactggtccattccagttggtggtttgacttctgactcttacgctggtcaaatcttctgggacgctgatgtctggatgcaacctggtttagttgctgctttcccagaatctgccaagagaattactaactaccgtactgctaagtactctcaagctttggaaaacgccaagactgcttacacttcctctcaaaaccaaacctctttctcttctgacgctgctatctactcttggacctccggtagatacggtaactgtaccgctaccggtccatgttgggactacgaataccacttgaacggtgatatcggtatctctttagttaaccaatgggttgtctccggtgacaatgaaactttcaagaacacccacttcccaatctacaactccattgctaccttgtacggtgatttgctaaagaagaacggttcttattacactttgactaacatgactgatccagacgaatacgctaacaatgtcgacgctggtggttacaccatgactttgatctctcaaaccttgtctaacgctaacgccttcagaaagcaattcggtatggacgaaaacaccacctggactgaaatggctgacaacatcttgttgatcagagaaaacgatgtcactttggaatacactaccatgaacaactctgttgctgttaagcaagctgacgttattttgtccacttttcctttggattacactaaaaactacactacctctgctgctttgaacgacttggactactacgctttgaagcaatctccagacggtccaggtatgacctacgctatcttctccatcgtcgccaacgacgtttctccatccggttgttctgcctatacttacgctcaatactcttacgacccatacatcagaggtccattcttccaattctccgaacaattattggatgattacactgccaacggtggtacccacccagccttcccattcttaaccggtcacggtggtgctaaccaagttgtcttatacggttacttgggtttgcgtttgttgccagatgacatgttgcacattgatccaaacctaccaccacaaattccatctgtcaagtacagaactttctactggagaggttggccaatccaagctgccagcaactacacccacaccactatccaaagagctacctccgttgctccattatccactgctgacccagtttacgccaacacctctatttccgtctctgttggtcaaaacactgctaactccactacttactctttgccagttaacggttctgctatcgttgttccaaacagacaaatcggttctattaacactgtcaccggtaacattgctcaatgtgtctctgtcttgtccaccgatgcttaccaaccaggtcaatacccaatctctgctgttgacggtgccgcttccactaagtggcaaccagaatttgctgctaatgtctcttctttgaccgttgacttgacctcttccaacgcatcctctgtttctggtttctacttcgattgggctcaagctccaccaactaacatcaccgtcttgttgcacaactcttcctccgccgctttgacatcttcctctacccatggtggttcctcctctgtctccttgaacatcaccacctccaacccttacgacgcttcctcctacgatgccaacgttatcgctttgtcctcctctaacaccactaactacactttcccagctccagtcgctaagccaagatacgctactttgttcgttcaaggtaaccaagccttggatgaaactgataccaaggctggtaacggtaccggtgctaccgttgccgaatgggctattctatcttaaattggcattcttcaatttgatagacacttatccctgcatattttttttataaacagcttatagactttcatgtaaatttttcctaattaatgtattatttacttcgttaattttccgttgaattattgacatgttaaaggtgcactaaatatacctaatacaaaaaatggttttctgtggcaaatatatacagtggaaatttcagcatataatccctgctttactctttccttaagatttcgtcataattagaactttttttggtaaactgcattttctaccattattactttacatatgtatagctacaaaactgtattttgaagtgaaaagtatgatgaataaatgaatcctcggtgttgcacacgaatacgacacttcccaatacactttgcctctccaagg

SEQ ID NO: 51 - DNA, 100 bp, synthetic construct
  misc_feature 1..100: note = INT59_FLANK5
  source 1..100: mol_type = other DNA
cgaaagcgttgaagcaaactacgtaaacaaacaaaaagtcaacatcctgtagatgtagctgcaaaaccacagaatactcgtgtgggagaaaatattgata

SEQ ID NO: 52 - DNA, 100 bp, synthetic construct
  misc_feature 1..100: note = INT59_FLANK3
  source 1..100: mol_type = other DNA
acctccgtcggctccggcgtacggcaccaaaaaatcccgcaataacttgacaccaatgggctaacggcgtccgcagaaccgtgacgcaatctgcggtttg

SEQ ID NO: 53 - DNA, 621 bp, synthetic construct
  misc_feature 1..621: note = Fragment containing the TDH3-promoter
  source 1..621: mol_type = other DNA
ggtctcggtgcttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgggagacc

SEQ ID NO: 54 - DNA, 621 bp, synthetic construct
  misc_feature 1..621: note = Fragment containing the PGK1-promoter
  source 1..621: mol_type = other DNA
ggtctcggtgcgggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgggagacc

SEQ ID NO: 55 - DNA, 621 bp, synthetic construct
  misc_feature 1..621: note = Fragment containing the ACT1-promoter
  source 1..621: mol_type = other DNA
ggtctcggtgcaacatatatacacaattacagtaacaataacaagaggacagatactaccaaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttctttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaacaatgggagacc

SEQ ID NO: 56 - DNA, 309 bp, synthetic construct
  misc_feature 1..309: note = Fragment containing PFY1-promoter (Sc_PFY1.pro and BsaI )
  source 1..309: mol_type = other DNA
ggtctcggtgcaggagacgttactttgtttatatatattagtatgtacaatcgcaaagaaatggagtgatgacatgttgtagtatttagtatgaggttactgtgtgggaggttttaccatgatttttggcgagaacacgccatgaaatgtctttgtacgaaactcattacccgcattaatattttttttctttttaaagctcagttgaccctttctcattcccttcttaaaacaactgtgtgatccttgagaaaagataaattacatacacaacataaacccaactacgatcgcaaataatgggagacc

SEQ ID NO: 57 - DNA, 307 bp, synthetic construct
  misc_feature 1..307: note = Fragment containing YKT6-promoter (Sc_YKT6.pro and BsaI)
  source 1..307: mol_type = other DNA
ggtctcggtgctttactgataatatacactctttggatcgagcccacttccagttggtaattggtgttccacaatttcagcattacatgtttttaaaccaaaattcggctccttttccctttttttcttattgggtggcgtgccgtacagaacgattggcttggtgtgaaatcaagagcaagcacaatagatatcaacatgaacaatatacaaaagtctctggcacagtttgactgcgttagaccaggctagggcatttctgaagctttacgtatcactagagaagttattttggcaatgggagacc

SEQ ID NO: 58 - DNA, 570 bp, synthetic construct
  misc_feature 1..570: note = Fragment containing the RPS3-promoter (Sc_RPS3.pro and BsaI)
  source 1..570: mol_type = other DNA
ggtctcggtgcaatccaagtaaaaggatggatatcgttatactaaaagcaacacagaaaaggtccacgtcagttccacacaataacatttacgtagtgttcacgcgaagcagttacatctcaactaacataattgctggtgagcctacaacactgcatgcgtaaacgtcaacgggattacgttagtatttttggccgccggtaaattctcttgtttttttttcttgatttcacttcttttcatgttcctttggaataatctaattcctcatgattaaatgagactgttttttgtttccgtaacatccatacctttcctgtataatattcttgctgtaaagtttgttttttttatgaaaaaaacattttcttttcttgagatgaggcgccgcgagcctttctcccatgggcagtggtaaattttccaaatcaatgcagctctttgaaatacaacagcatttttcatacattttaagcaatttctagtttgtagatattgttagattagtttttgaacattgttttgataactgaaaataaaacagcaaacaaactacaaaaatgggagacc

SEQ ID NO: 59 - DNA, 572 bp, synthetic construct
  misc_feature 1..572: note = Fragment containing the ZUO1-promoter (Sc_ZUO1.pro and BsaI)
  source 1..572: mol_type = other DNA
ggtctcggtgcctggcgttttatcttttatgcatccaatatctaatattacttccgatcacgcatttagttctgattacagcagaaatcgtagcgcgatgagacatttcatcaaatggccttttttttttgggcaatttttttatatcttgaaatgatagttgccttgtactttcaaccgttcatttcattaagaacttgactaaatatgaacatttcttaaaaaaaaggttgacatataaaaataatcgaatataaacgatggaatttttataaaattaaacacatatatatatatatattaactataaatatgtcaaagaaaccatacaatcatagatttataactatcttttggatgacattaatgaacataacgctcctaatacaaatgtccaaaaaatattacccgcaaatacgaatcttttttttttctcgatgaaattttgcaaagagttcgaaatttttatttcaagagctggtagagaaaatttcataaggttttcctaccgatgcttttataaaatcttcgttttgtctcacatataccaacaagagtaacaatgggagacc

SEQ ID NO: 60 - DNA, 574 bp, synthetic construct
  misc_feature 1..574: note = Fragment containing the MYO4-promoter (Sc_MYO4.pro and BsaI)
  source 1..574: mol_type = other DNA
ggtctcggtgcataacaatagaacaatagctttttgaccttccccttattttatttcaaaaggtaacagttagggctattcttaaactttctttagggtacacaatgaaaacatcaagattgagttaaaacccttaaaataaaactaggtctttaagaaactcactctccgggttatccatagaatgtttaccattctttagggttctgaactaatgcgaaaaaaaaaaaaggaaaagactgaaaaattcgaaaatatctcgaagtttagcagtagtagatgagaatagggtgtcttttatcgaaaacctgcaattgtaatacaacctttttttgaaagtcagcctttattctgattctgcatactcaattcctcaattcctacggagttatcacctttttttttaccttctcttcttttcttattgttctagctgaaaacattgttaccagtttggcgagacaatttattttcaatacgatacccttttgttcttctttttataattcaatctaattctaaaacacaaaaaaacaaaaaaaatcctataaccagttctcccgaatgggagacc

SEQ ID NO: 61 - DNA, 322 bp, synthetic construct
  misc_feature 1..322: note = Fragment containing ENO1 terminator
  source 1..322: mol_type = other DNA
ggtctcgtaaaagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcccctcggagacc

SEQ ID NO: 62 - DNA, 322 bp, synthetic construct
  misc_feature 1..322: note = Fragment containing ADH1-terminator (Sc_ADH1.ter and BsaI)
  source 1..322: mol_type = other DNA
ggtctcgtaaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacctcggagacc

SEQ ID NO: 63 - DNA, 372 bp, synthetic construct
  misc_feature 1..372: note = Fragment containing AQR1-terminator (Sc_AQR1.ter and BsaI)
  source 1..372: mol_type = other DNA
ggtctcgtaaattggcattcttcaatttgatagacacttatccctgcatattttttttataaacagcttatagactttcatgtaaatttttcctaattaatgtattatttacttcgttaattttccgttgaattattgacatgttaaaggtgcactaaatatacctaatacaaaaaatggttttctgtggcaaatatatacagtggaaatttcagcatataatccctgctttactctttccttaagatttcgtcataattagaactttttttggtaaactgcattttctaccattattactttacatatgtatagctacaaaactgtattttgaagtgaaaagtatgatgaataaatgaatcctcggagacc

SEQ ID NO: 64 - DNA, 322 bp, synthetic construct
  misc_feature 1..322: note = Fragment containing TDH1-terminator (Sc_TDH1.ter and BsaI)
  source 1..322: mol_type = other DNA
ggtctcgtaaaataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgccttcctcggagacc

SEQ ID NO: 65 - DNA, 322 bp, synthetic construct
  misc_feature 1..322: note = Fragment containing the CYC1-terminator
  source 1..322: mol_type = other DNA
ggtctcgtaaaacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagcttcgcagtttacactctcatcgtcctcggagacc

(19) United States
(12) Patent Application Publication
(10) Pub. No.: US 2024/0425888 A1
(43) Pub. Date: Dec. 26, 2024

(54) PROCESS FOR THE PRODUCTION OF ETHANOL AND RECOMBINANT YEAST CELL

(71) Applicant: DANISCO US INC., Palo Alto, CA (US)

(72) Inventors: Hans Marinus Charles Johannes DE BRUIJN, Echt (NL); Evert Tjeerd VAN RIJ, Echt (NL); Mickel Leonardus August JANSEN, Echt (NL); Marco Richard VAN DER WEERT, Echt (NL); Wouter KROES, Echt (NL); Johannes Gustaaf Ernst VAN LEEUWEN, Echt (NL)

(21) Appl. No.: 18/704,739

(22) PCT Filed: Nov. 4, 2022
(86) PCT No.: PCT/EP2022/080762

§ 371 Date: Apr. 25, 2024

(30) Foreign Application Priority Data
Nov. 4, 2021 (EP) 21206522.1

(51) Int. Cl. C12P 7/06; C12N 9/00; C12N 9/02; C12N 9/04; C12N 9/10; C12N 9/12; C12N 9/34; C12N 9/44; C12N 9/88; C12N 15/81

(52) U.S. Cl. CPC C12P 7/06; C12N 9/0006; C12N 9/0008; C12N 9/1029; C12N 9/1205; C12N 9/1217; C12N 9/2428; C12N 9/2451; C12N 9/88; C12N 9/93; C12N 15/81; C12Y 101/01001; C12Y 101/01006; C12Y 102/0101; C12Y 203/01008; C12Y 207/01019; C12Y 207/01028; C12Y 207/01029; C12Y 207/02012; C12Y 302/0101; C12Y 302/01021; C12Y 302/01028; C12Y 401/01039; C12Y 401/02009; C12Y 401/02022; C12Y 602/01001

(57) ABSTRACT

A process for the production of ethanol. comprising: fermentation of a feed. under anaerobic conditions. wherein the feed contains a di-saccharide. oligo-saccharide and/or poly-saccharide and wherein the fermentation is carried out in the presence of a recombinant yeast cell. which recombinant yeast produces a combination of proteins having glucosidase activity: and recovery of ethanol. and a recombinant yeast cell for use therein.

### FIELD OF THE INVENTION

The invention relates to a process for the production of ethanol and a recombinant yeast cell useful therein.

### BACKGROUND OF THE INVENTION

Microbial fermentation processes from renewable carbohydrate feedstocks are applied in the industrial production of a broad and rapidly expanding range of chemical compounds. Ethanol production by Saccharomyces cerevisiae is currently, by volume, the single largest fermentation process in industrial biotechnology. Various approaches have been proposed to improve the fermentative properties of organisms used in industrial biotechnology by genetic modification.

In literature several different approaches have been reported for ethanol production from starch-containing material.

Traditionally a multi-step process is applied, including both enzymatic hydrolysis and yeast-based fermentation. As a first step, amylase and glucoamylase enzyme can be added to the starch-containing media to produce glucose. The glucose can be converted in a yeast-based fermentation to ethanol. For example, US2017/0306310 in the name of Novozymes describes a process of producing a fermentation product, particularly ethanol, from starch-containing material comprising the steps of: (a) liquefying starch-containing material in the presence of an alpha amylase; (b) saccharifying the liquefied material; and (c) fermenting with a fermenting organism; wherein step (b) is carried out using at least a variant glucoamylase.

U.S. Pat. No. 10,227,613 in the name of Novozymes describes a process for producing fermentation products from starch-containing material comprising the steps of i) liquefying the starch-containing material using an alpha-amylase in the presence of a protease; ii) saccharifying the liquefied starch-containing material using a carbohydrate-source generating enzyme; and iii) fermenting using a fermenting organism, wherein a cellulolytic composition comprising two or more enzymes selected from the group consisting of an endoglucanase, a beta-glucosidase, a cellobiohydrolase, and a polypeptide having cellulolytic enhancing activity is present or added during fermentation or simultaneous saccharification and fermentation. The enzymes in U.S. Pat. No. 10,227,613 are generated ex-situ and added during fermentation or simultaneous saccharification and fermentation.

In an advancement, it was found that yeast can be transformed with a glucoamylase gene. WO 2020/043497 in the name of DSM describes a process for the production of ethanol comprising fermenting a corn slurry under anaerobic conditions in the presence of a recombinant yeast; and recovering the ethanol, wherein said recombinant yeast functionally expresses a heterologous nucleic acid sequence encoding a certain glucoamylase, wherein the process comprises dosing a glucoamylase at a concentration of 0.05 g/L or less.

Although good results are obtained with the above advanced process, further improvement is desirable.

Starch comprises amylose and amylopectin. Whilst amylose consists of linear chains of α-1-4 linked glucose, amylopectin is a glucose polymer in which the glucose residues are linked by either alpha-1,4 links or alpha-1,6 links. Glucoamylases are efficient in hydrolyzing the alpha-1,4 links, but traditionally glucoamylases have difficulties or are simply not capable of hydrolyzing the alpha-1,6 links, resulting in unfermentable oligosaccharides comprising such alpha-1,6 links.

WO2006/069289A2 describes a specific Trametes cingulata glucoamylase that was stated to have 4-7 fold higher alpha-1,6-debranching activity than other glucoamylases, such as Athelia rolfsii, Aspergillus niger and Talaromyces emersonii. It is mentioned that the claimed polynucleotide may be inserted into a host cell.

Jonathan et al, in their article titled “Characterization of branched gluco-oligosaccharides to study the mode-of-action of a glucoamylase from Hypocrea jecorina”, published in Carbohydrate Polymers Vol. 132 (2015), pages 59-66, describe a glucoamylase from Hypocrea jecorina that cleaves the alpha-1,4-linkage adjacent to the alpha-1,6-linkage at a lower rate than that of alpha-1,4-linkages in linear oligosaccharides, but is said to be more active on alpha-1,6-linkages than other glucoamylases.

It would be an advancement in the art to provide a process for the production of ethanol wherein no external glucosidases (i.e. proteins having glycosidic bond hydrolyzing activity) need to be added during fermentation. It would further be an advancement in the art to provide a recombinant yeast that would enable such a process.

### SUMMARY OF THE INVENTION

The inventors have now found a new process for the production of ethanol wherein no external glucosidases need to be added during fermentation. In addition, yeasts were found enabling such a process.

Accordingly the present invention provides a process for the production of ethanol, comprising: fermentation of a feed, under anaerobic conditions, wherein the feed contains a di-saccharide, oligo-saccharide and/or poly-saccharide and wherein the fermentation is carried out in the presence of a recombinant yeast cell, which recombinant yeast produces a combination of proteins having glucosidase activity; and recovery of ethanol.

In addition, the present invention provides a recombinant yeast cell useful in such a process. The invention therefore also provides a recombinant Saccharomyces yeast cell, preferably a recombinant Saccharomyces cerevisiae yeast cell, functionally expressing:
a first nucleotide sequence encoding a first protein having alpha-1,4-glucosidase activity; and
a further nucleotide sequence encoding a further protein having a glucosidase activity other than an alpha-1,4-glucosidase activity.

Use of the above recombinant yeast cell and/or the above process can advantageously result in reduction of the amount of ex-situ produced glucoamylase to be added during fermentation and may even allow one to completely refrain from dosing of glucoamylase during the fermentation.

That is, the use of the recombinant yeast cell according to the invention advantageously enables one to reduce the dosing of ex-situ produced or other external glucoamylase to the process by 10 to 100% whilst still allowing one to achieve a similar or even better ethanol yield and/or total residual sugar content at the end of fermentation.

### BRIEF DESCRIPTION OF THE SEQUENCE LISTING

This application contains a Sequence Listing in computer readable form, which is incorporated herein by reference. An overview is provided by Table 1 below.

TABLE 1
Overview of sequence listings:

| SEQ ID No: | Enzyme Name | Origin | Type |
|---|---|---|---|
| SEQ ID NO: 01 | alpha-1,4-glucosidase | Punctularia strigosozonata | protein |
| | (glucoamylase, Pstr_GA_0048) | | |
| SEQ ID NO: 02 | alpha-1,4-glucosidase | Punctularia strigosozonata | DNA |
| | (glucoamylase, Pstr_GA_0048) | | |
| SEQ ID NO: 03 | alpha-1,4-glucosidase | Punctularia strigosozonata | protein |
| | (glucoamylase, Pstr_GA_0009) | | |
| SEQ ID NO: 04 | alpha-1,4-glucosidase | Punctularia strigosozonata | DNA |
| | (glucoamylase, Pstr_GA_0009) | | |
| SEQ ID NO: 05 | alpha-1,4-glucosidase | Punctularia strigosozonata | protein |
| | (glucoamylase, Pstr_GA_0010) | | |
| SEQ ID NO: 06 | alpha-1,4-glucosidase | Punctularia strigosozonata | DNA |
| | (glucoamylase, Pstr_GA_0010) | | |
| SEQ ID NO: 07 | alpha-1,4-glucosidase | Punctularia strigosozonata | protein |
| | (glucoamylase, Pstr_GA_0033) | | |
| SEQ ID NO: 08 | alpha-1,4-glucosidase | Punctularia strigosozonata | DNA |
| | (glucoamylase, Pstr_GA_0033) | | |
| SEQ ID NO: 09 | alpha-1,4-glucosidase | Punctularia strigosozonata | protein |
| | (glucoamylase, Pstr_GA_0034) | | |
| SEQ ID NO: 10 | alpha-1,4-glucosidase | Punctularia strigosozonata | DNA |
| | (glucoamylase, Pstr_GA_0034) | | |
| SEQ ID NO: 11 | alpha-1,4-glucosidase | Punctularia strigosozonata | protein |
| | (glucoamylase) | | |
| SEQ ID NO: 12 | alpha-1,4-glucosidase | Punctularia strigosozonata | protein |
| | (glucoamylase with native signal | | |
| | sequence FGA09) | | |
| SEQ ID NO: 13 | alpha-1,4-glucosidase | Punctularia strigosozonata | protein |
| | (glucoamylase with Sc alpha mating | | |
| | factor signal sequence) | | |
| SEQ ID NO: 14 | apha-1,4-glucosidase | Myceliophthora sepedonium | protein |
| | (glucoamylase (mature)) | (Corynascus sepedonium) | |
| SEQ ID NO: 15 | alpha-1,4-glucosidase | Aspergillus niger | protein |
| | (glucoamylase (mature)) | | |
| SEQ ID NO: 16 | alpha-1,4-glucosidase | Trichoderma reesei | protein |
| | (glucoamylase (mature)) | | |
| SEQ ID NO: 17 | alpha-1,4-glucosidase | Botryotinia fuckeliana | protein |
| | (glucoamylase (mature)) | | |
| SEQ ID NO: 18 | alpha-1,4-glucosidase | Auricularia delicata | protein |
| | (glucoamylase (mature)) | | |
| SEQ ID NO: 19 | alpha-1,4-glucosidase | Talaromyces stipitatus | protein |
| | (glucoamylase (mature)) | | |
| SEQ ID NO: 20 | alpha-1,4-glucosidase | Piriformospora indica | protein |
| | (glucoamylase (mature)) | | |
| SEQ ID NO: 21 | alpha-1,4-glucosidase | Saccharomycopsis fibuligera | protein |
| | (glucoamylase (mature)) | | |
| SEQ ID NO: 22 | alpha-1,4-glucosidase | Saccharomyces cerevisiae | protein |
| | (glucoamylase (mature)) | | |
| SEQ ID NO: 23 | S. cerevisiae MATalpha signal | Artificial Sequence | DNA |
| | sequence | | |
| SEQ ID NO: 24 | alpha-1,6-glucosidase | Trametes coccinea | protein |
| | (debranching glucoamylase) | | |
| SEQ ID NO: 25 | alpha-1,6-glucosidase | Trametes coccinea | DNA |
| | (debranching glucoamylase) | | |
| SEQ ID NO: 26 | alpha-1,6-glucosidase | Hypocrea jecorina | protein |
| | (debranching glucoamylase) | | |
| SEQ ID NO: 27 | alpha-1,6-glucosidase | Hypocrea jecorina | DNA |
| | (debranching glucoamylase) | | |
| SEQ ID NO: 28 | alpha-1,6-glucosidase | Trametes cingulata | protein |
| | (debranching glucoamylase) | | |
| SEQ ID NO: 29 | alpha-1,6-glucosidase | Trametes cingulata | DNA |
| | (debranching glucoamylase) | | |
| SEQ ID NO: 30 | beta-glucosidase | Aspergillus aculeatus | protein |
| SEQ ID NO: 31 | beta-glucosidase | Aspergillus aculeatus | DNA |
| SEQ ID NO: 32 | beta-glucosidase | Aspergillus flavus | protein |
| SEQ ID NO: 33 | beta-glucosidase | Aspergillus flavus | DNA |
| SEQ ID NO: 34 | beta-glucosidase | Aspergillus kawachii | protein |
| SEQ ID NO: 35 | beta-glucosidase | Aspergillus kawachii | DNA |
| SEQ ID NO: 36 | alpha 1,1-glucosidase (trehalase) | Talaromyces cellulolyticus | protein |
| SEQ ID NO: 37 | alpha 1,1-glucosidase (trehalase) | Talaromyces cellulolyticus | DNA |
| SEQ ID NO: 38 | Connector 2J | Artificial Sequence | DNA |
| SEQ ID NO: 39 | Connector 2K | Artificial Sequence | DNA |
| SEQ ID NO: 40 | Connector 2L | Artificial Sequence | DNA |
| SEQ ID NO: 41 | Connector 2M | Artificial Sequence | DNA |
| SEQ ID NO: 42 | Connector 2N | Artificial Sequence | DNA |
| SEQ ID NO: 43 | Fragment A | Artificial Sequence | DNA |
| SEQ ID NO: 44 | Fragment B | Artificial Sequence | DNA |
| SEQ ID NO: 45 | Fragment C | Artificial Sequence | DNA |
| SEQ ID NO: 46 | Fragment D | Artificial Sequence | DNA |
| SEQ ID NO: 47 | Fragment E | Artificial Sequence | DNA |
| SEQ ID NO: 48 | Fragment F | Artificial Sequence | DNA |
| SEQ ID NO: 49 | Fragment G | Artificial Sequence | DNA |
| SEQ ID NO: 50 | Fragment H | Artificial Sequence | DNA |
| SEQ ID NO: 51 | INT59_FLANK5 | Artificial Sequence | DNA |
| SEQ ID NO: 52 | INT59_FLANK3 | Artificial Sequence | DNA |
| SEQ ID NO: 53 | Fragment containing the S. par. | Artificial Sequence | DNA |
| | TDH3-promoter (Spar_TDH3.pro) | | |
| SEQ ID NO: 54 | Fragment containing the S. | Artificial Sequence | DNA |
| | cerevisiae PGK1-promoter | | |
| | (Sc_PGK1.pro) | | |
| SEQ ID NO: 55 | Fragment containing the S. | Artificial Sequence | DNA |
| | cerevisiae ACT1 promoter | | |
| | (Sc_ACT1.pro) | | |
| SEQ ID NO: 56 | Fragment containing the S. | Artificial Sequence | DNA |
| | cerevisiae PFY1 promoter | | |
| | (Sc_PFY1.pro) | | |
| SEQ ID NO: 57 | Fragment containing the S. | Artificial Sequence | DNA |
| | cerevisiae YKT6 promoter | | |
| | (Sc_YKT6.pro) | | |
| SEQ ID NO: 58 | Fragment containing the S. | Artificial Sequence | DNA |
| | cerevisiae RPS3 promoter | | |
| | (Sc_RPS3.pro) | | |
| SEQ ID NO: 59 | Fragment containing the S. | Artificial Sequence | DNA |
| | cerevisiae ZUO1 promoter | | |
| | (Sc_ZUO1.pro) | | |
| SEQ ID NO: 60 | Fragment containing the S. | Artificial Sequence | DNA |
| | cerevisiae MYO4 promoter | | |
| | (Sc_MYO4.pro) | | |
| SEQ ID NO: 61 | Fragment containing the S. | Artificial Sequence | DNA |
| | cerevisiae ENO1-terminator | | |
| | (Sc_ENO1.ter) | | |
| SEQ ID NO: 62 | Fragment containing the S. | Artificial Sequence | DNA |
| | cerevisiae ADH1-terminator | | |
| | (Sc_ADH1.ter) | | |
| SEQ ID NO: 63 | Fragment containing the S. | Artificial Sequence | DNA |
| | cerevisiae QR1-terminator | | |
| | (Sc_AQR1.ter) | | |
| SEQ ID NO: 64 | Fragment containing the S. | Artificial Sequence | DNA |
| | cerevisiae TDH1-terminator | | |
| | (Sc_TDH1.ter) | | |
| SEQ ID NO: 65 | Fragment containing the S. | Artificial Sequence | DNA |
| | cerevisiae CYC1-terminator | | |
| | (Sc_CYC1.ter) | | |

### DEFINITIONS

Unless defined otherwise or clearly indicated by context, all technical and scientific terms used herein have the same meaning as commonly understood by one of ordinary skill in the art.

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element. When referring to a noun (e.g. a compound, an additive, etc.) in the singular, the plural is meant to be included. Thus, when referring to a specific moiety, e.g. “gene”, this means “at least one” of that gene, e.g. “at least one gene”, unless specified otherwise.

When referring to a compound of which several isomers exist (e.g. a D and an L enantiomer), the compound in principle includes all enantiomers, diastereomers and cis/trans isomers of that compound that may be used in the particular aspect of the invention; in particular when referring to such as compound, it includes the natural isomer(s).

Unless explicitly indicated otherwise, the various embodiments of the invention described herein can be cross-combined.

The term “carbon source” refers to a source of carbon, preferably a compound or molecule comprising carbon. Preferably the carbon source is a carbohydrate. A carbohydrate is understood herein to be an organic compound made of carbon, oxygen and hydrogen. Suitably the carbon source may be selected from the group consisting of mono-saccharides, di-saccharides, oligo-saccharides and/or poly-saccharides, acids and acid salts. More preferably the carbohydrates are mono-saccharides, di-saccharides, oligo-saccharides and/or poly-saccharides. By an oligo-saccharide is herein preferably understood a polymer comprising or consisting of 3 to 10 (mono-) saccharide units. Examples of mono-saccharides include glucose, fructose, galactose, arabinose and xylose. Examples of di-saccharides include sucrose, maltose, iso-maltose and trehalose. Examples of oligo-saccharides include maltotriose and panose.

The term “ferment”, and variations thereof such as “fermenting”, “fermentation” and/or “fermentative”, is used herein in a classical sense, i.e. to indicate that a process is or has been carried out under anaerobic conditions. An anaerobic fermentation is herein defined to be a fermentation carried out under anaerobic conditions. Anaerobic conditions are herein defined as conditions without any oxygen or in which essentially no oxygen is consumed by the yeast cell. Conditions in which essentially no oxygen is consumed suitably corresponds to an oxygen consumption of less than 5 mmol/l·h⁻¹, in particular to an oxygen consumption of less than 2.5 mmol/l·h⁻¹, or less than 1 mmol/l·h⁻¹. More preferably 0 mmol/L/h is consumed (i.e. oxygen consumption is not detectable). This suitably corresponds to a dissolved oxygen concentration in a culture broth of less than 5% of air saturation, more suitably to a dissolved oxygen concentration of less than 1% of air saturation, or less than 0.2% of air saturation.

The term “fermentation process” refers to a process for the preparation or production of a fermentation product.

The term “cell” refers to a eukaryotic or prokaryotic organism, preferably occurring as a single cell. In the present invention the cell is a recombinant yeast cell. That is, the recombinant cell is selected from the group of genera consisting of yeast.

The terms “yeast” and “yeast cell” are used herein interchangeably and refer to a phylogenetically diverse group of single-celled fungi, most of which are in the division of Ascomycota and Basidiomycota. The budding yeasts (“true yeasts”) are classified in the order Saccharomycesles. The yeast cell according to the invention is preferably a yeast cell derived from the genus of Saccharomyces. More preferably the yeast cell is a yeast cell of the species Saccharomyces cerevisiae.

The term “recombinant”, for example referring to a “recombinant yeast”, a “recombinant cell”, “recombinant micro-organism” and/or “recombinant strain” as used herein, refers to a yeast, cell, micro-organism or strain, respectively, containing nucleic acid which is the result of one or more genetic modifications. Simply put the yeast, cell, micro-organism or strain contains a different combination of nucleic acid from (either of) its parent(s). To construe a recombinant yeast, cell, micro-organism or strain, recombinant DNA technique(s) and/or another mutagenic technique(s) can be used. For example a recombinant yeast and/or a recombinant yeast cell may comprise nucleic acid not present in the corresponding wild-type yeast and/or cell, which nucleic acid has been introduced into that yeast and/or yeast cell using recombinant DNA techniques (i.e. a transgenic yeast and/or cell), or which nucleic acid not present in said wild-type yeast and/or cell is the result of one or more mutations—for example using recombinant DNA techniques or another mutagenesis technique such as UV-irradiation—in a nucleic acid sequence present in said wild-type yeast and/or yeast cell (such as a gene encoding a wild-type polypeptide) or wherein the nucleic acid sequence of a gene has been modified to target the polypeptide product (encoding it) towards another cellular compartment. Further, the term “recombinant” may suitably relate to a yeast, cell, micro-organism or strain from which nucleic acid sequences have been removed, for example using recombinant DNA techniques.

By a recombinant yeast comprising or having a certain activity is herein understood that the recombinant yeast may comprise one or more nucleic acid sequences encoding for a protein having such activity. Hence allowing the recombinant yeast to functionally express such a protein or enzyme.

The term “functionally expressing” means that there is a functioning transcription of the relevant nucleic acid sequence, allowing the nucleic acid sequence to actually be transcribed, for example resulting in the synthesis of a protein.

The term “transgenic” as used herein, for example referring to a “transgenic yeast” and/or a “transgenic cell”, refers to a yeast and/or cell, respectively, containing nucleic acid not naturally occurring in that yeast and/or cell and which has been introduced into that yeast and/or cell using for example recombinant DNA techniques, such as a recombinant yeast and/or cell.

The term “mutated” as used herein regarding proteins or polypeptides means that, as compared to the wild-type or naturally occurring protein or polypeptide sequence, at least one amino acid has been replaced with a different amino acid, inserted into, or deleted from the amino acid sequence. The replacement, insertion or deletion of the amino acid can for example be achieved via mutagenesis of nucleic acids encoding these amino acids. Mutagenesis is a well-known method in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989), published by Cold Spring Harbor Publishing).

The term “mutated” as used herein regarding genes means that, as compared to the wild-type or naturally occurring nucleic acid sequence, at least one nucleotide in the nucleic acid sequence of a gene or a regulatory sequence thereof, has been replaced with a different nucleotide, inserted into, or deleted from the nucleic acid sequence. The replacement, insertion or deletion of the amino acid can for example be achieved via mutagenesis, resulting for example in the transcription of a protein sequence with a qualitatively of quantitatively altered function or the knock-out of that gene. In the context of this invention an “altered gene” has the same meaning as a mutated gene.

The term “gen” or “gene”, as used herein, refers to a nucleic acid sequence that can be transcribed into mRNAs that are then translated into protein. A gene encoding for a certain protein refers to the one or more nucleic acid sequence(s) encoding for such a protein.

The term “nucleic acid” or “nucleotide” as used herein, refers to a monomer unit in a deoxyribonucleotide or ribonucleotide polymer, i.e. a polynucleotide, in either single or double-stranded form, and unless otherwise limited, encompasses known analogues having the essential nature of natural nucleotides in that they hybridize to single-stranded nucleic acids in a manner similar to naturally occurring nucleotides (e. g., peptide nucleic acids). For example, a certain enzyme that is defined by a nucleotide sequence encoding the enzyme, includes (unless otherwise limited) the nucleotide sequence hybridising to the reference nucleotide sequence encoding the enzyme. A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “nucleotide sequence” and “nucleic acid sequence” are used interchangeably herein. An example of a nucleic acid sequence is a DNA sequence.

The terms “polypeptide”, “peptide” and “protein” are used interchangeably herein to refer to a polymer of amino acid residues, for example illustrated by an amino acid sequence. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulphation, gamma-carboxylation of glutamic acid residues, hydroxylation and ADP-ribosylation.

The term “enzyme” refers herein to a protein having a catalytic function. Where a protein catalyzes a certain biological reaction, the terms “protein” and “enzyme” may be used interchangeable herein. When an enzyme is mentioned with reference to an enzyme class (EC), the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at http://www.chem.qmul.ac.uk/iubmb/enzyme/. Other suitable enzymes that have not (yet) been classified in a specified class but may be classified as such, are meant to be included.

If referred herein to a protein or a nucleic acid sequence, such as a gene, by reference to a accession number, this number in particular is used to refer to a protein or nucleic acid sequence (gene) having a sequence as can be found via www.ncbi.nlm.nih.gov/, (as available on 1 Oct. 2020) unless specified otherwise.

Every nucleic acid sequence herein that encodes a polypeptide also includes any conservatively modified variants thereof. This includes that, by reference to the genetic code, it describes every possible silent variation of the nucleic acid. The term “conservatively modified variants” applies to both amino acid and nucleic acid sequences. With respect to particular nucleic acid sequences, conservatively modified variants refers to those nucleic acids which encode identical or conservatively modified variants of the amino acid sequences due to the degeneracy of the genetic code. The term “degeneracy of the genetic code” refers to the fact that a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation.

The term “functional homologue” (or in short “homologue”) of a polypeptide and/or amino acid sequence having a specific sequence (e.g. “SEQ ID NO: X”), as used herein, refers to a polypeptide and/or amino acid sequence comprising said specific sequence with the proviso that one or more amino acids are mutated, substituted, deleted, added, and/or inserted, and which polypeptide has (qualitatively) the same enzymatic functionality for substrate conversion.

The term “functional homologue” (or in short “homologue”) of a polynucleotide and/or nucleic acid sequence having a specific sequence (e.g. “SEQ ID NO: X”), as used herein, refers to a polynucleotide and/or nucleic acid sequence comprising said specific sequence with the proviso that one or more nucleic acids are mutated, substituted, deleted, added, and/or inserted, and which polynucleotide encodes for a polypeptide sequence that has (qualitatively) the same enzymatic functionality for substrate conversion. With respect to nucleic acid sequences, the term functional homologue is meant to include nucleic acid sequences which differ from another nucleic acid sequence due to the degeneracy of the genetic code and encode the same polypeptide sequence.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences.

Amino acid or nucleotide sequences are said to be homologous when exhibiting a certain level of similarity. Two sequences being homologous indicate a common evolutionary origin. Whether two homologous sequences are closely related or more distantly related is indicated by “percent identity” or “percent similarity”, which is high or low respectively. Although disputed, to indicate “percent identity” or “percent similarity”, “level of homology” or “percent homology” are frequently used interchangeably. A comparison of sequences and determination of percent identity between two sequences can be accomplished using a mathematical algorithm. The skilled person will be aware of the fact that several different computer programs are available to align two sequences and determine the homology between two sequences (Kruskal et al., “An overview of sequence comparison: Time warps, string edits, and macromolecules”, (1983), Society for Industrial and Applied Mathematics (SIAM), Vol 25, No. 2, pages 201-237 and D. and the handbook edited by Sankoff and J. B. Kruskal, (ed.), “Time warps, string edits and macromolecules: the theory and practice of sequence comparison”, (1983), pp. 1-44, published by Addison-Wesley Publishing Company, Massachusetts USA).

The percent identity between two amino acid sequences can be determined using the Needleman and Wunsch algorithm for the alignment of two sequences. (Needleman et al “A General Method Applicable to the Search for Similarities in the Amino Acid Sequence of Two Proteins” (1970) J. Mol. Biol. Vol. 48, pages 443-453). The algorithm aligns amino acid sequences as well as nucleotide sequences. The Needleman-Wunsch algorithm has been implemented in the computer program NEEDLE. For the purpose of this invention the NEEDLE program from the EMBOSS package is used (version 2.8.0 or higher, see Rice et al, “EMBOSS: The European Molecular Biology Open Software Suite” (2000), Trends in Genetics vol. 16, (6) pages 276-277, http://emboss.bioinformatics.nl/). For protein sequences, EBLOSUM62 is used for the substitution matrix. For nucleotide sequences, EDNAFULL is used. Other matrices can be specified. The optional parameters used for alignment of amino acid sequences are a gap-open penalty of 10 and a gap extension penalty of 0.5. The skilled person will appreciate that all these different parameters will yield slightly different results but that the overall percentage identity of two sequences is not significantly altered when using different algorithms.

The homology or identity is the percentage of identical matches between the two full sequences over the total aligned region including any gaps or extensions. The homology or identity between the two aligned sequences is calculated as follows: Number of corresponding positions in the alignment showing an identical amino acid in both sequences divided by the total length of the alignment including the gaps. The identity defined as herein can be obtained from NEEDLE and is labelled in the output of the program as “IDENTITY”.

The homology or identity between the two aligned sequences is calculated as follows: Number of corresponding positions in the alignment showing an identical amino acid in both sequences divided by the total length of the alignment after subtraction of the total number of gaps in the alignment. The identity defined as herein can be obtained from NEEDLE by using the NOBRIEF option and is labelled in the output of the program as “longest-identity”.

A variant of a nucleotide or amino acid sequence disclosed herein may also be defined as a nucleotide or amino acid sequence having one or more mutations, substitutions, insertions and/or deletions as compared to the nucleotide or amino acid sequence specifically disclosed herein (e.g. in de the sequence listing).

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. In an embodiment, conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. In an embodiment, conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

Nucleotide sequences of the invention may also be defined by their capability to hybridise with parts of specific nucleotide sequences disclosed herein, respectively, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity. Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6 x SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

“Expression” refers to the transcription of a gene into structural RNA (rRNA, tRNA) or messenger RNA (mRNA) with subsequent translation into a protein.

“Overexpression” refers to expression of a gene, respectively a nucleic acid sequence, by a recombinant cell in excess to its expression in a corresponding wild-type cell. Such overexpression can for example be arranged for by: increasing the frequency of transcription of one or more nucleic acid sequences, for example by operational linking of the nucleic acid sequence to a promoter functional within the recombinant cell; and/or by increasing the number of copies of a certain nucleic acid sequence.

The terms “upregulate”, “upregulated” and “upregulation” refer to a process by which a cell increases the quantity of a cellular component, such as RNA or protein. Such an upregulation may be in response to or caused by a genetic modification.

By the term “pathway” or “metabolic pathway” is herein understood a series of chemical reactions in a cell that build and breakdown molecules.

Nucleic acid sequences (i.e. polynucleotides) or proteins (i.e. polypeptides) may be native or heterologous to the genome of the host cell.

“Native”, “homologous” or “endogenous” with respect to a host cell, means that the nucleic acid sequence does naturally occur in the genome of the host cell or that the protein is naturally produced by that cell. The terms “native”, “homologous” and “endogenous” are used interchangeable herein.

As used herein, “heterologous” may refer to a nucleic acid sequence or a protein. For example, “heterologous”, with respect to the host cell, may refer to a polynucleotide that does not naturally occur in that way in the genome of the host cell or that a polypeptide or protein is not naturally produced in that manner by that cell. A heterologous nucleic acid sequence is a nucleic acid that originates from a foreign species, or, if from the same species, is substantially modified from its native form in composition and/or genomic locus by deliberate human intervention. For example, a promoter operably linked to a native structural gene is from a species different from that from which the structural gene is derived, or, if from the same species, one or both are substantially modified from their original form. A heterologous protein may originate from a foreign species or, if from the same species, is substantially modified from its original form by deliberate human intervention. That is, heterologous protein expression involves expression of a protein that is not naturally expressed in that way in the host cell. The term “heterologous expression” refers to the expression of heterologous nucleic acids in a host cell. The expression of heterologous proteins in eukaryotic host cell systems such as yeast are well known to those of skill in the art. A polynucleotide comprising a nucleic acid sequence of a gene encoding a certain protein or enzyme with a specific activity can be expressed in such a eukaryotic system. In some embodiments, transformed/transfected cells may be employed as expression systems for the expression of the enzymes. Expression of heterologous proteins in yeast is well known. Sherman, F., et al., Methods in Yeast Genetics, (1986), published by Cold Spring Harbor Laboratory, is a well-recognized work describing the various methods available to express proteins in yeast. Two widely utilized yeasts are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

As used herein “promoter” is a DNA sequence that directs the transcription of a (structural) gene or other (part of) nucleic acid sequence. Suitably, a promoter is located in the 5′-region of a gene, proximal to the transcriptional start site of a (structural) gene. Promoter sequences may be constitutive, inducible or repressible. In an embodiment there is no (external) inducer needed.

The term “vector” as used herein, includes reference to an autosomal expression vector and to an integration vector used for integration into the chromosome.

The term “expression vector” refers to a DNA molecule, linear or circular, that comprises a segment encoding a polypeptide of interest under the control of (i.e. operably linked to) additional nucleic acid segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both. In particular an expression vector comprises a nucleic acid sequence that comprises in the 5′ to 3′ direction and operably linked: (a) a yeast-recognized transcription and translation initiation region, (b) a coding sequence for a polypeptide of interest, and (c) a yeast-recognized transcription and translation termination region.

“Plasmid” refers to autonomously replicating extrachromosomal DNA which is not integrated into a microorganism's genome and is usually circular in nature.

An “integration vector” refers to a DNA molecule, linear or circular, that can be incorporated in a microorganism's genome and provides for stable inheritance of a gene encoding a polypeptide of interest. The integration vector generally comprises one or more segments comprising a gene sequence encoding a polypeptide of interest under the control of (i.e. operably linked to) additional nucleic acid segments that provide for its transcription. Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is nonfunctional in that organism. Integration of the segment comprising the gene of interest may be selected if an appropriate marker is included within that segment.

By “host cell” is herein understood a cell, such as a yeast cell, that is to be transformed with one or more nucleic acid sequences encoding for one or more heterologous proteins, to construe a transformed cell, also referred to as a recombinant cell. For example, the transformed cell may contain a vector and may support the replication and/or expression of the vector.

“Transformation” and “transforming”, as used herein, refers to the insertion of an exogenous polynucleotide into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host cell genome. “Transformation” and “transforming”, as used herein, refers to the insertion of an exogenous polynucleotide (i.e. an exogenous nucleic acid sequence) into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host cell genome.

By “constitutive expression” and “constitutively expressing” is herein understood that there is a continuous transcription of a nucleic acid sequence. That is, the nucleic acid sequence is transcribed in an ongoing manner. Constitutively expressed genes are always “on”.

By “anaerobic constitutive expression” is herein understood that nucleic acid sequence is constitutively expressed in an organism under anaerobic conditions. That is, under anaerobic conditions the nucleic acid sequence is transcribed in an ongoing manner, i.e. under such anaerobic conditions the genes are always “on”.

By “disruption” is herein understood any disruption of activity, including, but not limited to, deletion, mutation and reduction of the affinity of the disrupted gene and expression of RNA complementary to such disrupted gene. It includes all nucleic acid modifications such as nucleotide deletions or substitutions, gene knock-outs, and other actions which affect the translation or transcription of the corresponding polypeptide and/or which affect the enzymatic (specific) activity, its substrate specificity, and/or or stability. It also includes modifications that may be targeted on the coding sequence or on the promotor of the gene. A gene disruptant is a cell that has one or more disruptions of the respective gene. Native to yeast herein is understood as that the gene is present in the yeast cell before the disruption.

The term “encoding” has the same meaning as “coding for”. Thus, by way of example, “one or more genes encoding a transketolase” has the same meaning as “one or more genes coding for a transketolase”.

As far as genes or nucleic acid sequences encoding a protein or an enzyme are concerned, the phrase “one or more nucleic acid sequences encoding a X”, wherein X denotes a protein, has the same meaning as “one or more nucleic acid sequences encoding a protein having X activity”. Thus, by way of example, “one or more nucleic acid sequences encoding a transketolase” has the same meaning as “one or more nucleic acid sequences encoding a protein having transketolase activity”.

The abbreviation “NADH” refers to reduced, hydrogenated form of nicotinamide adenine dinucleotide. The abbreviation “NAD+” refers to the oxidized form of nicotinamide adenine dinucleotide. Nicotinamide adenine dinucleotide may act as a so-called cofactor, assisting in biochemical reactions and/or transformations in a cell.

“NADH dependent” or “NAD+ dependent” is herein equivalent to NADH specific and “NADH dependency” or “NAD+ dependency” is herein equivalent to NADH specificity.

By a “NADH dependent” or “NAD+ dependent” enzyme is herein understood an enzyme that is exclusively depended on NADH/NAD+ as a co-factor or that is predominantly dependent on NADH/NAD+ as a cofactor, i.e. as contrasted to other types of co-factor. By an “exclusive NADH/NAD+ dependent” enzyme is herein understood an enzyme that has an absolute requirement for NADH/NAD+ over NADPH/NADP+. That is, it is only active when NADH/NAD+ is applied as cofactor. By a “predominantly NADH/NDA+-dependent” enzyme is herein understood an enzyme that has a higher specificity and/or a higher catalytic efficiency for NADH/NAD+ as a cofactor than for NADPH/NADP+ as a cofactor.

The enzyme's specificity characteristics can be described by the formula:

$$1 < {K}_{m} {NADP}^{+} / {K}_{m} {NAD}^{+} < ∞ ( infinity )$$

wherein Kₘ is the so-called Michaelis constant.

For a predominantly NADH-dependent enzyme, preferably KₘNADP⁺/KₘNAD⁺ is between 1 and 1000, between 1 and 500, between 1 and 200, between 1 and 100, between 1 and 50, between 1 and 10, between 5 and 100, between 5 and 50, between 5 and 20 or between 5 and 10.

The Kₘ 's for the enzymes herein can be determined as enzyme specific, for NAD⁺ and NADP⁺ respectively, using know analysis techniques, calculations and protocols. These are described for instance in Lodish et al., Molecular Cell Biology 6^{th} Edition, Ed. Freeman, pages 80 and 81, e.g. FIG. 3-22. For an predominantly NADH-dependent enzyme, preferably the ratio of the catalytic efficiency for NADPH/NADP+ as a cofactor (k_{cat}/Kₘ)^{NADP+} to NADH/NAD+ as cofactor (k_{cat}/Kₘ)^{NAD+}, i.e. the catalytic efficiency ratio (k_{cat}/Kₘ)^{NADP+}:(k_{cat}/Kₘ)^{NAD+}, is more than 1:1, more preferably equal to or more than 2:1, still more preferably equal to or more than 5:1, even more preferably equal to or more than 10:1, yet even more preferably equal to or more than 20:1, even still more preferably equal to or more than 100:1, and most preferably equal to or more than 1000:1. There is no upper limit, but for practical reasons the predominantly NADH-dependent enzyme may have a catalytic efficiency ratio (k_{cat}/Kₘ)^{NADP+}:(k_{cat}/Kₘ)^{NAD+} of equal to or less than 1.000.000.000:1 (i.e. 1.10⁹:1).

### The Yeast Cell

The recombinant yeast cell is preferably a yeast cell, or derived from a yeast cell, from the genus of Saccharomycesceae or the genus of Schizosaccharomycesaceae. That is, preferably the host cell from which the recombinant yeast cell is derived is a yeast cell from the genus of Saccharomycesceae or the genus of Schizosaccharomycesceae.

Examples of suitable yeast cells include Saccharomyces, such as Saccharomyces cerevisiae, Saccharomyces eubayanus, Saccharomyces jurei, Saccharomyces pastorianus, Saccharomyces beticus, Saccharomyces fermentati, Saccharomyces paradoxus, Saccharomyces uvarum and Saccharomyces bayanus.

Examples of suitable yeast cells further include Schizosaccharomyces, such as Schizosaccharomyces pombe, Schizosaccharomyces japonicus, Schizosaccharomyces octosporus and Schizosaccharomyces cryophilus.

Other exemplary yeasts include Torulaspora such as Torulaspora delbrueckii; Kluyveromyces such as Kluyveromyces marxianus; Pichia such as Pichia stipitis, Pichia pastoris or Pichia angusta; Zygosaccharomyces such as Zygosaccharomyces bailii; Brettanomyces such as Brettanomyces inter medius; Brettanomyces bruxellensis, Brettanomyces anomalus, Brettanomyces custersianus, Brettanomyces naardenensis, Brettanomyces nanus, Dekkera bruxellensis and Dekkera anomala; Metschmkowia, Issatchenkia, such as Issatchenkia orientalis, Kloeckera such as Kloeckera apiculata; and Aureobasidium such as Aureobasidium pullulans.

The yeast cell is preferably a yeast cell of the genus Schizosaccharomyces, herein also referred to as a Schizosaccharomyces yeast cell, or a yeast cell of the genus Saccharomyces, herein also referred to as a Saccharomyces yeast cell. More preferably the yeast cell is a yeast cell derived from a yeast cell of the species Saccharomyces cerevisiae, herein also referred to as a Saccharomyces cerevisae yeast cell. That is, preferably the host cell from which the recombinant yeast cell is derived is a yeast cell from the species Saccharomyces cerevisiae. Hence, preferably the recombinant yeast cell is a recombinant Saccharomyces yeast cell, more preferably a recombinant Saccharomyces cerevisiae yeast cell.

Preferably the yeast cell is an industrial yeast cell. The living environments of yeast cells in industrial processes are significantly different from that in the laboratory. Industrial yeast cells must be able to perform well under multiple environmental conditions which may vary during the process. Such variations include changes in nutrient sources, pH, ethanol concentration, temperature, oxygen concentration, etc., which together have potential impact on the cellular growth and ethanol production of the yeast cell. An industrial yeast cell can be understood to refer to a yeast cell that, when compared to a laboratory counterpart, has a more robust performance. That is, when compared to a laboratory counterpart, the industrial yeast cell shows less variation in performance when one or more environmental conditions selected from the group of nutrient sources, pH, ethanol concentration, temperature, oxygen concentration, are varied during fermentation. Preferably, the yeast cell is constructed on the basis of an industrial yeast cell as a host, wherein the construction is conducted as described hereinafter. Examples of industrial yeast cells are Ethanol Red® (Fermentis) Fermiol® (DSM) and Thermosacc® (Lallemand).

The recombinant yeast cell described herein may be derived from any host cell capable of producing a fermentation product. Preferably the host cell is a yeast cell, more preferably an industrial yeast cell as described herein above. Preferably the yeast cell described herein is derived from a host cell having the ability to produce ethanol.

The yeast cell described herein may be derived from the host cell through any technique known by one skilled in the art to be suitable therefore. Such techniques may include any one or more of mutagenesis, recombinant DNA technology (including, but not limited to, CRISPR-CAS techniques), selective and/or adaptive evolution, mating, cell fusion, and/or cytoduction between yeast strains. Suitably the one or more desired genes are incorporated in the yeast cell by a combination of one or more of the above techniques.

The recombinant yeast may be subjected to evolutionary engineering to improve its properties. Evolutionary engineering processes are known processes. Evolutionary engineering is a process wherein industrially relevant phenotypes of a microorganism, herein the recombinant yeast, can be coupled to the specific growth rate and/or the affinity for a nutrient, by a process of rationally set-up natural selection. Evolutionary Engineering is for instance described in detail in Kuijper, M, et al, FEMS, Eukaryotic cell Research 5 (2005) 925-934, WO2008/041840 and WO2009/112472. After the evolutionary engineering the resulting pentose fermenting recombinant cell is isolated. The isolation may be executed in any known manner, e.g. by separation of cells from a recombinant cell broth used in the evolutionary engineering, for instance by taking a cell sample or by filtration or centrifugation.

In an embodiment, the recombinant yeast is marker-free. As used herein, the term “marker” refers to a gene encoding a trait or a phenotype which permits the selection of, or the screening for, a host cell containing the marker. Marker-free means that markers are essentially absent in the recombinant yeast. Being marker-free is particularly advantageous when antibiotic markers have been used in construction of the recombinant yeast and are removed thereafter. Removal of markers may be done using any suitable prior art technique, e.g. intramolecular recombination.

In one embodiment, the recombinant yeast is constructed on the basis of an inhibitor tolerant host cell, wherein the construction is conducted as described hereinafter. Inhibitor tolerant host cells may be selected by screening strains for growth on inhibitors containing materials, such as illustrated in Kadar et al, Appl. Biochem. Biotechnol. (2007), Vol. 136-140, 847-858, wherein an inhibitor tolerant S. cerevisiae strain ATCC 26602 was selected.

The recombinant yeast cells according to the invention are thus preferably inhibitor tolerant, i.e. they can withstand common inhibitors at the level that they typically have with common pretreatment and hydrolysis conditions, so that the recombinant yeast cells can find broad application, i.e. it has high applicability for different feedstock, different pretreatment methods and different hydrolysis conditions. In an embodiment the recombinant yeast cell is inhibitor tolerant. Inhibitor tolerance is resistance to inhibiting compounds. The presence and level of inhibitory compounds in lignocellulose may vary widely with variation of feedstock, pretreatment method hydrolysis process. Examples of categories of inhibitors are carboxylic acids, furans and/or phenolic compounds. Examples of carboxylic acids are lactic acid, acetic acid or formic acid. Examples of furans are furfural and hydroxy-methylfurfural. Examples or phenolic compounds are vannilin, syringic acid, ferulic acid and coumaric acid. The typical amounts of inhibitors are for carboxylic acids: several grams per liter, up to 20 grams per liter or more, depending on the feedstock, the pretreatment and the hydrolysis conditions. For furans: several hundreds of milligrams per liter up to several grams per liter, depending on the feedstock, the pretreatment and the hydrolysis conditions. For phenolics: several tens of milligrams per liter, up to a gram per liter, depending on the feedstock, the pretreatment and the hydrolysis conditions.

In an embodiment, the recombinant yeast cell is a cell that is naturally capable of alcoholic fermentation, preferably, anaerobic alcoholic fermentation. A recombinant yeast cell preferably has a high tolerance to ethanol, a high tolerance to low pH (i.e. capable of growth at a pH lower than about 5, about 4, about 3, or about 2.5) and towards organic and/or a high tolerance to elevated temperatures.

### Combination of Proteins Having Glucosidase Activity

A protein, respectively an enzyme, having glucosidase activity is herein also referred to as a “glucosidase”. The term glucosidase as used herein preferably refers to a protein, respectively an enzyme, that can catalyse the hydrolysis of a di-saccharide, oligo-saccharide and/or poly-saccharide. That is, preferably a glucosidase is herein understood to be a protein, respectively enzyme, having glycosidic bond hydrolyzing activity. More preferably a glucosidase is a protein, respectively an enzyme, that catalyses the hydrolysis, also referred to as cleavage, of a di-saccharide, oligo-saccharide and/or poly-saccharide comprising two or more mono-saccharide units connected via a glycosidic bond.

Glucosidases are also referred to as “glycoside hydrolases”, “glycosidase hydrolase” or “glycosyl hydrolases” and are mentioned within the “glycosidase” enzyme class of EC 3.2.1. The terms “glycosidase”, “glycoside hydrolase”, “glycosyl hydrolase”, “glycosidase hydrolase” and “glucosidase” are therefore used interchangeably herein.

By a glycosidic bond is herein preferably understood a so-called O-glycosidic bond, binding one monosaccharide unit (also referred to as a one sugar unit) to another monosaccharide unit in a saccharide comprising two or more monosaccharide units.

As indicated above, glucosidases can for example be found within enzyme class E.C. 3.2.1.

More preferably the glucosidases are glycosidases that catalyse the hydrolysis of glucose linkages (also referred to as glucose bonds) between glucose units in a di-saccharide, oligo-saccharide or polysaccharide, preferably removing or releasing successive glucose units from such di-saccharide, oligo-saccharide or polysaccharide. More preferably the glucosidases are glucosidases classified within enzyme classes E.C. 3.2.1.1, E.C. 3.2.1.2, E.C. 3.2.1.3, E.C. 3.2.1.4, E.C. 3.2.1.6, E.C. 3.2.1.9, E.C. 3.2.1.10, E.C. 3.2.1.20, E.C. 3.2.1.21, E.C. 3.2.1.28, E.C. 3.2.1.33, E.C. 3.2.1.41, E.C. 3.2.1.70 and/or E.C. 3.2.1.74. Most preferably the glucosidases are glucosidases classified within enzyme classes E.C. 3.2.1.3, E.C. 3.2.1.10, E.C. 3.2.1.21 and/or E.C. 3.2.1.28.

The combination of proteins having glucosidase activity can suitably comprise two, three, four, five, six or more proteins having glucosidase activity. More preferably the combination of proteins having glucosidase activity comprises two, three, four, five, six or more proteins classified within enzyme classes E.C. 3.2.1.1, E.C. 3.2.1.2, E.C. 3.2.1.3, E.C. 3.2.1.4, E.C. 3.2.1.6, E.C. 3.2.1.9, E.C. 3.2.1.10, E.C. 3.2.1.20, E.C. 3.2.1.21, E.C. 3.2.1.28, E.C. 3.2.1.33, E.C. 3.2.1.41, E.C. 3.2.1.70 and/or E.C. 3.2.1.74. Most preferably the combination of proteins having glucosidase activity comprises two, three, four, five, six or more proteins that are classified within enzyme classes classified within enzyme classes E.C. 3.2.1.3, E.C. 3.2.1.10, E.C. 3.2.1.21 and/or E.C. 3.2.1.28.

Preferably the combination of proteins having glucosidase activity is a combination of two, three or four proteins chosen from the group consisting of a protein having alpha 1,4-glucosidase activity (preferably within E.C. 3.2.1.3); a protein having alpha 1,6-glucosidase activity (preferably within E.C. 3.2.1.10); a protein having beta-glucosidase activity (preferably within E.C. 3.2.1.21); and a protein having alpha 1,1-glucosidase activity (preferably within E.C. 3.2.1.28).

Preferably the combination of proteins comprises a first protein having alpha-1,4-glucosidase activity; and a further protein having a glucosidase activity other than an alpha-1,4-glucosidase activity.

More preferably the combination of proteins having glucosidase activity is a combination of
a first protein having alpha 1,4-glucosidase activity (preferably within E.C. 3.2.1.3); and
a further protein having alpha 1,6-glucosidase activity (preferably within E.C. 3.2.1.10); and/or a further protein having beta-glucosidase activity (preferably within E.C. 3.2.1.21); and/or a further protein having alpha 1,1-glucosidase activity (preferably within E.C. 3.2.1.28). Most preferably the combination of proteins having glucosidase activity is a combination of a first protein having alpha 1,4-glucosidase activity (preferably within E.C. 3.2.1.3); and a further protein having alpha 1,6-glucosidase activity (preferably within E.C. 3.2.1.10); and a further protein having beta-glucosidase activity (preferably within E.C. 3.2.1.21); and a further protein having alpha 1,1-glucosidase activity (preferably within E.C. 3.2.1.28).

Preferably the process is thus a process wherein the recombinant yeast cell, preferably a recombinant Saccharomyces yeast cell, and most preferably a recombinant Saccharomyces cerevisiae yeast cell, produces a combination of:
a first protein having alpha 1,4-glucosidase activity (preferably within E.C. 3.2.1.3); and
a further protein having alpha 1,6-glucosidase activity (preferably within E.C. 3.2.1.10); and/or a further protein having beta-glucosidase activity (preferably within E.C. 3.2.1.21); and/or a further protein having alpha 1,1-glucosidase activity (preferably within E.C. 3.2.1.28).

Most preferably the process is a process wherein the recombinant yeast cell, preferably a recombinant Saccharomyces yeast cell, and most preferably a recombinant Saccharomyces cerevisiae yeast cell, produces a combination of a first protein having alpha 1,4-glucosidase activity (preferably within E.C. 3.2.1.3); and a further protein having alpha 1,6-glucosidase activity (preferably within E.C. 3.2.1.10); and a further protein having beta-glucosidase activity (preferably within E.C. 3.2.1.21); and a further protein having alpha 1,1-glucosidase activity (preferably within E.C. 3.2.1.28).

Herein below these glucosidases will be discussed in further detail.

As explained below, the recombinant yeast can advantageously further produce
a protein comprising phosphoketolase activity (EC 4.1.2.9 or EC 4.1.2.22); and/or
a protein having phosphotransacetylase (PTA) activity (EC 2.3.1.8); and/or
a protein having acetate kinase (ACK) activity (EC 2.7.2.12); and/or
a protein having ribulose-1,5-biphosphate carboxylase oxygenase (Rubisco) activity; and/or
a protein having phosphoribulokinase (PRK) activity; and/or
a protein comprising NADH dependent acetylating acetaldehyde dehydrogenase activity; and/or
a protein comprising acetyl-CoA synthetase activity; and/or
a protein comprising alcohol dehydrogenase activity; and/or
a protein having glycerol dehydrogenase activity (E.C. 1.1.1.6); and/or
a protein having dihydroxyacetone kinase activity (E.C. 2.7.1.28 or E.C. 2.7.1.29); and/or
a protein having glycerol transporter activity.

In analogy to the above the recombinant yeast cell according to the invention is preferably a recombinant Saccharomyces yeast cell, more preferably a Saccharomyces cerevisiae yeast cell, functionally expressing:
a first nucleotide sequence encoding a first protein having alpha-1,4-glucosidase activity; and
a further nucleotide sequence encoding a further protein having a glucosidase activity other than an alpha-1,4-glucosidase activity.

More preferably the recombinant yeast cell is a recombinant Saccharomyces yeast cell, more preferably a Saccharomyces cerevisiae yeast cell, functionally expressing:
a first nucleotide sequence encoding a first protein having alpha-1,4-glucosidase activity; and
a further nucleotide sequence encoding a further protein having alpha-1,6-glucosidase activity and/or a further nucleotide sequence encoding a further protein having alpha-1,1-glucosidase activity and/or a further nucleotide sequence encoding a further protein having beta-glucosidase activity.

Most preferably the recombinant yeast cell is a recombinant Saccharomyces yeast cell, more preferably a Saccharomyces cerevisiae yeast cell, functionally expressing:
a first nucleotide sequence encoding a first protein having alpha-1,4-glucosidase activity; and
a further nucleotide sequence encoding a further protein having alpha-1,6-glucosidase activity; and
a further nucleotide sequence encoding a further protein having alpha-1,1-glucosidase activity; and
a further nucleotide sequence encoding a further protein having beta-glucosidase activity.

Advantageously the recombinant yeast cell is a recombinant Saccharomyces yeast cell, more preferably a Saccharomyces cerevisiae yeast cell, that further functionally expresses:
a nucleotide sequence encoding a protein comprising phosphoketolase activity (EC 4.1.2.9 or EC 4.1.2.22); and/or
a nucleotide sequence encoding a protein having phosphotransacetylase (PTA) activity (EC 2.3.1.8); and/or
a nucleotide sequence encoding a protein having acetate kinase (ACK) activity (EC 2.7.2.12); and/or
a nucleotide sequence encoding a protein having ribulose-1,5-biphosphate carboxylase oxygenase (Rubisco) activity; and/or
a nucleotide sequence encoding a protein having phosphoribulokinase (PRK) activity; and/or
a nucleotide sequence encoding a protein comprising NADH dependent acetylating acetaldehyde dehydrogenase activity; and/or
a nucleotide sequence encoding a protein comprising acetyl-CoA synthetase activity; and/or
a nucleotide sequence encoding a protein comprising alcohol dehydrogenase activity; and/or
a nucleotide sequence encoding a protein having glycerol dehydrogenase activity (E.C. 1.1.1.6); and/or
a nucleotide sequence encoding a protein having dihydroxyacetone kinase activity (E.C. 2.7.1.28 or E.C. 2.7.1.29); and/or
a nucleotide sequence encoding a protein having glycerol transporter activity.

The above recombinant yeast cells, preferably recombinant Saccharomyces yeast cells, more preferably recombinant Saccharomyces cerevisiae yeast cells, can advantageously be used in the process according to the invention.

### Alpha 1,4-Glucosidase

An alpha 1,4-glucosidase can suitably be understood to be a protein, suitably an enzyme, having alpha-1,4-glycosidic bond hydrolyzing activity. More preferably it is understood to be a protein, suitably an enzyme, that catalyses the hydrolysis of (1->4)-linkages in di-saccharides, oligo-saccharides and/or poly-saccharides, removing successive glucose units.

Such a protein, respectively enzyme, can also be referred to herein as a protein, respectively enzyme, having “glucan 1,4-alpha glucosidase” activity or “glucoamylase” activity or simply as “glucan 1,4-alpha glucosidase” or “alpha-1,4-glucosidase” or “glucoamylase”. The above wording is used herein interchangeably. Preferably the protein having alpha-1,4-glycosidic bond hydrolyzing activity is a protein within enzyme class E.C. 3.2.1.3. Suitably the protein may have other or further activities. Preferably, however, the alpha 1,4-glucosidase activity is dominating.

More preferably the alpha 1,4-glucosidase is a protein, respectively an enzyme, which protein, respectively which enzyme, comprises or has:
an amino acid sequence of SEQ ID NO: 01, SEQ ID NO:03, SEQ ID NO: 05, SEQ ID NO: 07, SEQ ID NO: 09, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21 or SEQ ID NO: 22; or
an amino acid sequence which has at least 70% sequence identity, preferably at least 75%, 80%, 85%, 90%, 95, 98%, or 99% sequence identity with the amino acid sequence of SEQ ID NO: 01, SEQ ID NO: 03, SEQ ID NO: 05, SEQ ID NO: 07, SEQ ID NO: 09, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21 and/or SEQ ID NO: 22.

Most preferably the alpha 1,4-glucosidase is a protein, respectively an enzyme, which protein, respectively which enzyme, comprises or has:
an amino acid sequence of SEQ ID NO: 01, SEQ ID NO:03, SEQ ID NO: 05, SEQ ID NO: 07 or SEQ ID NO: 09; or
an amino acid sequence which has at least 70% sequence identity, preferably at least 75%, 80%, 85%, 90%, 95, 98%, or 99% sequence identity with the amino acid sequence of SEQ ID NO: 01, SEQ ID NO: 03, SEQ ID NO: 05, SEQ ID NO: 07 and/or SEQ ID NO: 09.

Preferably, a recombinant yeast cell functionally expressing an alpha 1,4-glucosidase, preferably comprises a nucleotide sequence encoding a protein having alpha-1,4-glycosidic bond hydrolyzing activity, which protein comprises or has
an amino acid sequence of SEQ ID NO: 01, SEQ ID NO:03, SEQ ID NO: 05, SEQ ID NO: 07, SEQ ID NO: 09, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21 or SEQ ID NO: 22, more preferably SEQ ID NO: 01, SEQ ID NO:03, SEQ ID NO: 05, SEQ ID NO: 07 or SEQ ID NO: 09; or
an amino acid sequence which has at least 70% sequence identity, preferably at least 75%, 80%, 85%, 90%, 95, 98%, or 99% sequence identity with the amino acid sequence of SEQ ID NO: 01, SEQ ID NO: 03, SEQ ID NO: 05, SEQ ID NO: 07, SEQ ID NO: 09, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21 and/or SEQ ID NO: 22, more preferably SEQ ID NO: 01, SEQ ID NO:03, SEQ ID NO: 05, SEQ ID NO: 07 and/or SEQ ID NO: 09.
   The nucleotide sequence can be a native or heterologous nucleotide sequence and is preferably a heterologous nucleotide sequence.

A protein can be defined by its amino acid sequence. In addition, a protein can be further defined by a nucleotide sequence. As explained in detail above under definitions, a certain protein that is defined by a nucleotide sequence encoding the protein, includes (unless otherwise limited) the nucleotide sequence hybridising to such nucleotide sequence encoding the protein.

The nucleotide sequence encoding the protein having alpha-1,4-glycosidic bond hydrolyzing activity is preferably a nucleotide sequence of SEQ ID NO: 02, SEQ ID NO: 04, SEQ ID NO: 06 or SEQ ID NO: 08 or a nucleotide sequence having at least 70% sequence identity, preferably at least 75%, 80%, 85%, 90%, 95, 98%, or 99% sequence identity with the nucleotide sequence of SEQ ID NO: 02, SEQ ID NO: 04, SEQ ID NO: 06 and/or SEQ ID NO: 08.

A recombinant yeast cell functionally expressing an alpha 1,4-glucosidase, therefore preferably comprises a nucleotide sequence of SEQ ID NO: 02, SEQ ID NO: 04, SEQ ID NO: 06 or SEQ ID NO: 08 or a nucleotide sequence having at least 70% sequence identity, preferably at least 75%, 80%, 85%, 90%, 95, 98%, or 99% sequence identity with the nucleotide sequence of SEQ ID NO: 02, SEQ ID NO: 04, SEQ ID NO: 06 and/or SEQ ID NO: 08.

A signal sequence (also referred to as signal peptide, targeting signal, localization signal, localization sequence, transit peptide, leader sequence or leader peptide) can be present at the N-terminus of a polypeptide, where it signals that the polypeptide is to be excreted, for example outside the cell and into the media.

Preferably the nucleotide sequence(s) encoding the protein having alpha-1,4-glycosidic bond hydrolyzing activity is codon optimized and any native signal sequences are replaced by those of the host cell. As indicated above, recombinant yeast host cells from the species Saccharomyces cerevisiae are preferred. Therefore, preferably the nucleotide sequence encoding the glucoamylase is codon optimized and any native signal sequences are replaced by the S. cerevisiae MATalpha signal sequence, more preferably the S. cerevisiae MATalpha signal nucleotide sequence of SEQ ID NO: 23

The recombinant yeast cell may comprise one, two, or more copies of the nucleotide sequence encoding the protein having alpha-1,4-glycosidic bond hydrolyzing activity. Suitably the recombinant yeast cell can comprise in the range from equal to or more than 1, preferably equal to or more than 2 to equal to or less than 30, preferably equal to or less than 20 and most preferably equal to or less than 10 copies of the nucleotide sequence encoding the protein having alpha-1,4-glycosidic bond hydrolyzing activity. Most preferably the recombinant yeast cell may comprise one, two, three, four, five, six, seven, eight, nine, ten, eleven or twelve copies of the nucleotide sequence encoding the protein having alpha-1,4-glycosidic bond hydrolyzing activity.

In a preferred embodiment, the activity of the alpha-1,4-glucosidase described above is fine-tuned or upregulated by overexpression. Preferably the nucleotide sequence encoding the alpha-1,4-glucosidase is preceded by a promoter, the alpha-1,4-glucosidase promoter.

The promoter can be a native promoter, a heterologous promoter or a synthetic promoter. Preferably the recombinant yeast cell is a recombinant Saccharomyces cerevisiae yeast cell and preferably the alpha-1,4-glucosidase promoter is a promoter that is native to Saccharomyces cerevisiae.

More preferably the alpha-1,4-glucosidase promoter is selected from the list consisting of: pTDH3, pPGK1, pHTA1, pTEF1, pPGK1, pPRS3, pYKT6, pACT1, pZOU1, pMYO4 and pPFY1, or a functional homologue thereof comprising a nucleotide sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity therewith. The alpha-1,4-glucosidase promoter advantageously enables higher expression of the alpha-1,4-glucosidase, preferably by a multiplication factor of 2 or more.

### Alpha 1,6-Glucosidase

An alpha 1,6-glucosidase can suitably be understood to be a protein, suitably an enzyme, having alpha-1,6-glycosidic bond hydrolyzing activity. Preferably it is understood to be a protein, respectively an enzyme that can release an alpha-1->6-linked glucose. Such a protein, respectively enzyme, can also be referred to herein as a protein, respectively enzyme, having “glucan 1,6-alpha glucosidase” activity, “oligo-1,6-glucosidase” activity or “debranching glucoamylase” activity or simply as “glucan 1,6-alpha glucosidase”, “oligo-1,6-glucosidase” or “alpha-1,6-glucosidase” or “debranching glucoamylase”. The above wording is used herein interchangeably. Preferably the protein having alpha-1,6-glycosidic bond hydrolyzing activity is a protein within enzyme class E.C. 3.2.1.10. Suitably the protein may have other or further activities. Preferably, however, the alpha 1,6-glucosidase activity is dominating.

More preferably the alpha 1,6-glucosidase is a protein, respectively an enzyme, which protein, respectively which enzyme, comprises or has:
an amino acid sequence of SEQ ID NO: 24, SEQ ID NO: 26 or SEQ ID NO: 28; or
an amino acid sequence which has at least 70% sequence identity, preferably at least 75%, 80%, 85%, 90%, 95, 98%, or 99% sequence identity with the amino acid sequence of SEQ ID NO: 24, SEQ ID NO: 26 and/or SEQ ID NO: 28.

Preferably, a recombinant yeast cell functionally expressing an alpha 1,6-glucosidase, preferably comprises a nucleotide sequence encoding a protein having alpha-1,6-glycosidic bond hydrolyzing activity, which protein comprises or has
an amino acid sequence of SEQ ID NO: 24, SEQ ID NO: 26 or SEQ ID NO: 28; or
an amino acid sequence which has at least 70% sequence identity, preferably at least 75%, 80%, 85%, 90%, 95, 98%, or 99% sequence identity with the amino acid sequence of SEQ ID NO: 24, SEQ ID NO: 26 and/or SEQ ID NO: 28.
   The nucleotide sequence can be a native or heterologous nucleotide sequence and is preferably a heterologous nucleotide sequence.

A protein can be defined by its amino acid sequence. In addition, a protein can be further defined by a nucleotide sequence. As explained in detail above under definitions, a certain protein that is defined by a nucleotide sequence encoding the protein, includes (unless otherwise limited) the nucleotide sequence hybridising to such nucleotide sequence encoding the protein.

The nucleotide sequence encoding the protein having alpha-1,6-glycosidic bond hydrolyzing activity is preferably a nucleotide sequence of SEQ ID NO: 25, SEQ ID NO: 27 or SEQ ID NO: 29 or a nucleotide sequence having at least 70% sequence identity, preferably at least 75%, 80%, 85%, 90%, 95, 98%, or 99% sequence identity with the nucleotide sequence of SEQ ID NO: 25, SEQ ID NO: 27 and/or SEQ ID NO: 29.

A recombinant yeast cell functionally expressing an alpha 1,6-glucosidase, therefore preferably comprises a nucleotide sequence of SEQ ID NO: 25, SEQ ID NO: 27 or SEQ ID NO: 29 or SEQ ID NO: 08 or a nucleotide sequence having at least 70% sequence identity, preferably at least 75%, 80%, 85%, 90%, 95, 98%, or 99% sequence identity with the nucleotide sequence of SEQ ID NO: 25, SEQ ID NO: 27 and/or SEQ ID NO: 29.

A signal sequence (also referred to as signal peptide, targeting signal, localization signal, localization sequence, transit peptide, leader sequence or leader peptide) can be present at the N-terminus of a polypeptide, where it signals that the polypeptide is to be excreted, for example outside the cell and into the media.

Preferably the nucleotide sequence(s) encoding the protein having alpha-1,6-glycosidic bond hydrolyzing activity is codon optimized and any native signal sequences are replaced by those of the host cell. As indicated above, recombinant yeast host cells from the species Saccharomyces cerevisiae are preferred. Therefore, preferably the nucleotide sequence encoding the glucoamylase is codon optimized and any native signal sequences are replaced by the S. cerevisiae MATalpha signal sequence, more preferably the S. cerevisiae MATalpha signal nucleotide sequence of SEQ ID NO: 23

The recombinant yeast cell may comprise one, two, or more copies of the nucleotide sequence encoding the protein having alpha-1,6-glycosidic bond hydrolyzing activity. Suitably the recombinant yeast cell can comprise in the range from equal to or more than 1, preferably equal to or more than 2 to equal to or less than 30, preferably equal to or less than 20 and most preferably equal to or less than 10 copies of the nucleotide sequence encoding the protein having alpha-1,6-glycosidic bond hydrolyzing activity. Most preferably the recombinant yeast cell may comprise one, two, three, four, five, six, seven, eight, nine, ten, eleven or twelve copies of the nucleotide sequence encoding the protein having alpha-1,6-glycosidic bond hydrolyzing activity.

In a preferred embodiment, the activity of the alpha-1,6-glucosidase described above is fine-tuned or upregulated by overexpression. Preferably the nucleotide sequence encoding the alpha-1,6-glucosidase is preceded by a promoter, the alpha-1,6-glucosidase promoter.

The promoter can be a native promoter, a heterologous promoter or a synthetic promoter. Preferably the recombinant yeast cell is a recombinant Saccharomyces cerevisiae yeast cell and preferably the alpha-1,6-glucosidase promoter is a promoter that is native to Saccharomyces cerevisiae.

More preferably the alpha-1,6-glucosidase promoter is selected from the list consisting of: pTDH3, pPGK1, pHTA1, pTEF1, pPGK1, pPRS3, pYKT6, pACT1, pZOU1, pMYO4 and pPFY1, or a functional homologue thereof comprising a nucleotide sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity therewith. The alpha-1,6-glucosidase promoter advantageously enables higher expression of the alpha-1,6-glucosidase, preferably by a multiplication factor of 2 or more.

### Beta-Glucosidase

A beta-glucosidase can suitably be understood to be a protein, suitably an enzyme, having beta-1,2-glycosidic bond hydrolyzing activity, beta-1,3-glycosidic bond hydrolyzing activity, beta-1,4-glycosidic bond hydrolyzing activity and/or beta-1,6-glycosidic bond hydrolyzing activity. More preferably the beta-glucosidase is a beta-glucosidase at least having beta-1,4-glycosidic bond hydrolyzing activity. More preferably the beta-glucosidase can catalyse the hydrolysis of a beta-glycosidic bond (for example beta-1,2-glycosidic linkage, beta-1,3-glycosidic linkage, beta-1,4-glycosidic linkage and/or beta-1,6-glycosidic linkage) in a di-saccharide, oligosaccharide and/or polysaccharide. Such a protein, respectively enzyme, can also be referred to herein as a protein, respectively enzyme, having “glucan-beta glucosidase” activity or “beta-glucosidase” activity or simply as “glucan-beta glucosidase” or “beta-glucosidase”. The above wording is used herein interchangeably. Preferably the protein having beta-glycosidic bond hydrolyzing activity is a protein within enzyme class E.C. 3.2.1.21. Suitably the protein may have other or further activities. Preferably, however, the beta-glucosidase activity is dominating.

The beta-glucosidase can be a protein having beta-1,2-glucosidase activity, beta-1,3-glucosidase activity, beta-1,4-glucosidase activity and/or beta-1,6-glucosidase activity. Preferably the beta-glucosidase has at least beta-1,4-glucosidase activity. Such a protein having at least beta-1,4-glucosidase activity is herein also referred to as a beta-1,4-glucosidase.

More preferably the beta-glucosidase is a protein, respectively an enzyme, which protein, respectively which enzyme, comprises or has:
an amino acid sequence of SEQ ID NO: 30, SEQ ID NO: 32 or SEQ ID NO: 34, more preferably of SEQ ID NO: 34; or
an amino acid sequence which has at least 70% sequence identity, preferably at least 75%, 80%, 85%, 90%, 95, 98%, or 99% sequence identity with the amino acid sequence of SEQ ID NO: 30, SEQ ID NO: 32 and/or SEQ ID NO: 34, more preferably of SEQ ID NO 34.

Preferably, a recombinant yeast cell functionally expressing an beta-glucosidase, preferably comprises a nucleotide sequence encoding a protein having beta-glycosidic bond hydrolyzing activity, which protein comprises or has
an amino acid sequence of SEQ ID NO: 30, SEQ ID NO: 32 or SEQ ID NO: 34, more preferably of SEQ ID NO 34; or
an amino acid sequence which has at least 70% sequence identity, preferably at least 75%, 80%, 85%, 90%, 95, 98%, or 99% sequence identity with the amino acid sequence of SEQ ID NO: 30, SEQ ID NO: 32 and/or SEQ ID NO: 34, more preferably of SEQ ID NO 34.
   The nucleotide sequence can be a native or heterologous nucleotide sequence and is preferably a heterologous nucleotide sequence.

A protein can be defined by its amino acid sequence. In addition, a protein can be further defined by a nucleotide sequence. As explained in detail above under definitions, a certain protein that is defined by a nucleotide sequence encoding the protein, includes (unless otherwise limited) the nucleotide sequence hybridising to such nucleotide sequence encoding the protein.

The nucleotide sequence encoding the protein having beta-glycosidic bond hydrolyzing activity is preferably a nucleotide sequence of SEQ ID NO: 31, SEQ ID NO: 33 or SEQ ID NO: 35, more preferably of SEQ ID NO 35; or a nucleotide sequence having at least 70% sequence identity, preferably at least 75%, 80%, 85%, 90%, 95, 98%, or 99% sequence identity with the nucleotide sequence of SEQ ID NO: 31, SEQ ID NO: 33 and/or SEQ ID NO: 35, more preferably of SEQ ID NO 35.

A recombinant yeast cell functionally expressing an beta-glucosidase, therefore preferably comprises a nucleotide sequence of SEQ ID NO: 31, SEQ ID NO: 33 or SEQ ID NO: 35, more preferably of SEQ ID NO 35; or a nucleotide sequence having at least 70% sequence identity, preferably at least 75%, 80%, 85%, 90%, 95, 98%, or 99% sequence identity with the nucleotide sequence of SEQ ID NO: 31, SEQ ID NO: 33 and/or SEQ ID NO: 35, more preferably of SEQ ID NO 35.

A signal sequence (also referred to as signal peptide, targeting signal, localization signal, localization sequence, transit peptide, leader sequence or leader peptide) can be present at the N-terminus of a polypeptide, where it signals that the polypeptide is to be excreted, for example outside the cell and into the media.

Preferably the nucleotide sequence(s) encoding the protein having beta-glycosidic bond hydrolyzing activity is codon optimized and any native signal sequences are replaced by those of the host cell. As indicated above, recombinant yeast host cells from the species Saccharomyces cerevisiae are preferred. Therefore, preferably the nucleotide sequence encoding the glucoamylase is codon optimized and any native signal sequences are replaced by the S. cerevisiae MATbeta signal sequence, more preferably the S. cerevisiae MATbeta signal nucleotide sequence of SEQ ID NO: 23

The recombinant yeast cell may comprise one, two, or more copies of the nucleotide sequence encoding the protein having beta-glycosidic bond hydrolyzing activity. Suitably the recombinant yeast cell can comprise in the range from equal to or more than 1, preferably equal to or more than 2 to equal to or less than 30, preferably equal to or less than 20 and most preferably equal to or less than 10 copies of the nucleotide sequence encoding the protein having beta-glycosidic bond hydrolyzing activity. Most preferably the recombinant yeast cell may comprise one, two, three, four, five, six, seven, eight, nine, ten, eleven or twelve copies of the nucleotide sequence encoding the protein having beta-glycosidic bond hydrolyzing activity.

In a preferred embodiment, the activity of the beta-glucosidase described above is fine-tuned or upregulated by overexpression. Preferably the nucleotide sequence encoding the beta-glucosidase is preceded by a promoter, the beta-glucosidase promoter.

The promoter can be a native promoter, a heterologous promoter or a synthetic promoter. Preferably the recombinant yeast cell is a recombinant Saccharomyces cerevisiae yeast cell and preferably the beta-glucosidase promoter is a promoter that is native to Saccharomyces cerevisiae.

More preferably the beta-glucosidase promoter is selected from the list consisting of: pTDH3, pPGK1, pHTA1, pTEF1, pPGK1, pPRS3, pYKT6, pACT1, pZOU1, pMYO4 and pPFY1, or a functional homologue thereof comprising a nucleotide sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity therewith. The beta-glucosidase promoter advantageously enables higher expression of the beta-glucosidase, preferably by a multiplication factor of 2 or more.

### Alpha 1,1-Glucosidase

An alpha 1,1-glucosidase can suitably be understood to be a protein, suitably an enzyme, having alpha, alpha-1,1-glycosidic bond hydrolyzing activity. Such a protein, respectively enzyme, can also be referred to herein as a protein, respectively enzyme, having “glucan 1,1-alpha glucosidase” activity or “alpha, alpha trehalase” activity or “alpha, alpha trehalose glucohydrolase” activity or simply as “glucan 1,1-alpha glucosidase” or “alpha-1,1-glucosidase” or “alpha, alpha trehalase” or “alpha, alpha trehalose glucohydrolase” or even simply as “trehalase”. The above wording is used herein interchangeably. Preferably the protein having alpha-1,1-glycosidic bond hydrolyzing activity is a protein within enzyme class E.C. 3.2.1.28. Suitably the protein may have other or further activities. Preferably, however, the alpha 1,1-glucosidase activity is dominating.

More preferably the alpha 1,1-glucosidase is a protein, respectively an enzyme, which protein, respectively which enzyme, comprises or has:
an amino acid sequence of SEQ ID NO: 36; or
an amino acid sequence which has at least 70% sequence identity, preferably at least 75%, 80%, 85%, 90%, 95, 98%, or 99% sequence identity with the amino acid sequence of SEQ ID NO: 36.

Preferably, a recombinant yeast cell functionally expressing an alpha 1,1-glucosidase, preferably comprises a nucleotide sequence encoding a protein having alpha-1,1-glycosidic bond hydrolyzing activity, which protein comprises or has
an amino acid sequence of SEQ ID NO: 36; or
an amino acid sequence which has at least 70% sequence identity, preferably at least 75%, 80%, 85%, 90%, 95, 98%, or 99% sequence identity with the amino acid sequence of SEQ ID NO: 36. The nucleotide sequence can be a native or heterologous nucleotide sequence and is preferably a heterologous nucleotide sequence.

A protein can be defined by its amino acid sequence. In addition, a protein can be further defined by a nucleotide sequence. As explained in detail above under definitions, a certain protein that is defined by a nucleotide sequence encoding the protein, includes (unless otherwise limited) the nucleotide sequence hybridising to such nucleotide sequence encoding the protein. The nucleotide sequence encoding the protein having alpha-1,1-glycosidic bond hydrolyzing activity is preferably a nucleotide sequence of SEQ ID NO: 37 or a nucleotide sequence having at least 70% sequence identity, preferably at least 75%, 80%, 85%, 90%, 95, 98%, or 99% sequence identity with the nucleotide sequence of SEQ ID NO: 37.

A recombinant yeast cell functionally expressing an alpha 1,1-glucosidase, therefore preferably comprises a nucleotide sequence of SEQ ID NO: 37 or a nucleotide sequence having at least 70% sequence identity, preferably at least 75%, 80%, 85%, 90%, 95, 98%, or 99% sequence identity with the nucleotide sequence of SEQ ID NO: 37.

A signal sequence (also referred to as signal peptide, targeting signal, localization signal, localization sequence, transit peptide, leader sequence or leader peptide) can be present at the N-terminus of a polypeptide, where it signals that the polypeptide is to be excreted, for example outside the cell and into the media.

Preferably the nucleotide sequence(s) encoding the protein having alpha-1,1-glycosidic bond hydrolyzing activity is codon optimized and any native signal sequences are replaced by those of the host cell. As indicated above, recombinant yeast host cells from the species Saccharomyces cerevisiae are preferred. Therefore, preferably the nucleotide sequence encoding the glucoamylase is codon optimized and any native signal sequences are replaced by the S. cerevisiae MATalpha signal sequence, more preferably the S. cerevisiae MATalpha signal nucleotide sequence of SEQ ID NO: 23

The recombinant yeast cell may comprise one, two, or more copies of the nucleotide sequence encoding the protein having alpha-1,1-glycosidic bond hydrolyzing activity. Suitably the recombinant yeast cell can comprise in the range from equal to or more than 1, preferably equal to or more than 2 to equal to or less than 30, preferably equal to or less than 20 and most preferably equal to or less than 10 copies of the nucleotide sequence encoding the protein having alpha-1,1-glycosidic bond hydrolyzing activity. Most preferably the recombinant yeast cell may comprise one, two, three, four, five, six, seven, eight, nine, ten, eleven or twelve copies of the nucleotide sequence encoding the protein having alpha-1,1-glycosidic bond hydrolyzing activity.

In a preferred embodiment, the activity of the alpha 1,1-glucosidase described above is fine-tuned or upregulated by overexpression. Preferably the nucleotide sequence encoding the alpha 1,1-glucosidase is preceded by a promoter, the alpha 1,1-glucosidase promoter.

The promoter can be a native promoter, a heterologous promoter or a synthetic promoter. Preferably the recombinant yeast cell is a recombinant Saccharomyces cerevisiae yeast cell and preferably the alpha 1,1-glucosidase promoter is a promoter that is native to Saccharomyces cerevisiae.

More preferably the alpha 1,1-glucosidase promoter is selected from the list consisting of: pTDH3, pPGK1, pHTA1, pTEF1, pPGK1, pPRS3, pYKT6, pACT1, pZOU1, pMYO4 and pPFY1, or a functional homologue thereof comprising a nucleotide sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity therewith. The alpha 1,1-glucosidase promoter advantageously enables higher expression of the alpha 1,1-glucosidase, preferably by a multiplication factor of 2 or more.

### Dosing of External Glucosidase.

By the term “dosing” is herein understood the ex-situ addition of (external) glucosidase. That is, such glucosidase is not produced in-situ by a recombinant yeast cell during fermentation, but is rather produced ex-situ, outside of the fermentation process. The glucosidase is preferably a glucosidase within enzyme class E.C. 3.2.1. Such external glucosidase can be added, in addition to the glucosidase that is already produced in-situ by the recombinant yeast cell(s) that is/are functionally expressing glucosidase.

Preferably the process according to the invention therefore comprises dosing of an ex-situ produced protein having glucosidase activity at a concentration of 0.05 g/L or less, calculated as the total amount of such protein in grams per liter of feed. The recombinant yeast cell(s) and processes according to the invention advantageously allow one to severely reduce and even avoid the addition of ex-situ produced (i.e. external) glucosidase. More preferably the dosing of such external glucosidase is reduced to a concentration of equal to or less than 0.05 g/L, more preferably equal to or less than 0.04 g/L, still more preferably equal to or less than 0.02 g/L, even more preferably equal to or less than 0.01 g/L and most preferably equal to or less than 0.005 g/L or even equal to or less than 0.001 g/L, calculated as the total amount of external glucosidase in grams per liter of feed. Such feed can suitably be a saccharide composition, such as a corn slurry. For example, ex-situ produced glucosidase, preferably as a liquid product, may be dosed in an amount equal to or less than 0.05 grams per one kilo corn slurry, preferably in an amount equal to or less than 0.005 grams per one kilo corn slurry.

For example, ex-situ produced glucosidase can be dosed at a concentration between 0.005 and 0.05 g/L (gram per liter), between 0.01 and 0.05 g/L, between 0.02 and 0.05 g/L, between 0.03 and 0.05 g/L, or between 0.04 and 0.05 g/L, calculated as the total amount of glucosidase in grams per liter of feedstock. Or such ex-situ produced glucosidase can be dosed at concentration between 0.005 and 0.04 g/L, between 0.01 and 0.04 g/L, between 0.02 and 0.04 g/L, or between 0.03 and 0.04 g/L, calculated as the total amount of glucosidase in grams per liter of feedstock.

Suitably such ex-situ produced glucosidase can be dosed at a concentration between 0.005 and 0.04 g/L, between 0.005 and 0.03 g/L, between 0.005 and 0.02 g/L, or between 0.005 and 0.01 g/L, calculated as the total amount of glucosidase in grams per liter of feedstock.

Preferably the process of the invention is carried out without dosing any external glucosidase during fermentation. That is, preferably the process is a process, wherein no ex-situ produced protein having glucosidase activity is dosed during fermentation. Hence, the dosage of ex-situ produced glucosidase during fermentation is preferably zero.

The skilled person knows how to dose glucosidase. If glucosidase is dosed to the fermentation, glucosidase can be dosed separately, before or after adding a recombinant yeast cell. Glucosidase can be dosed as a dry product, e.g. as powder or a granulate, or as a liquid. Glucosidase can be dosed together with other components such as antibiotics. Glucosidase can also be dosed as part of the back set, i.e. a stream in which part of the thin stillage is recycled e.g. to the fermentation. Glucosidase can also be dosed using a combination of these methods.

### Redox Sink

Preferably the recombinant yeast cell can further comprise one or more genetic modifications to functionally express a protein that functions in a metabolic pathway forming a non-native redox sink.

For example, these one or more genetic modifications can be one or more genetic modifications for the functional expression of one or more, optionally heterologous, nucleic acid sequences encoding for one or more NAD+/NADH dependent proteins that function in a metabolic pathway to convert NADH to NAD+. Several examples of such metabolic pathways exist, as illustrated further below.

WO2014/081803 describes a recombinant microorganism expressing a heterologous phosphoketolase, phosphotransacetylase or acetate kinase and bifunctional acetaldeyde-alcohol dehydrogenase, incorporated herein by reference; and WO2015/148272 describes a recombinant S. cerevisiae strain expressing a heterologous phosphoketolase, phosphotransacetylase and acetylating acetaldehyde dehydrogenase, incorporated herein by reference. Further WO2018172328A1 describes a recombinant cell that may comprise one or more (heterologous) genes coding for an enzyme having phosphoketolase activity. The phosphoketalase (PKL) routes described in WO2014/081803, WO2015/148272 and WO2018172328A1, provide preferred metabolic pathways to convert NADH to NAD+ and the NADH dependent phosphoketolase described therein is a preferred NADH dependent protein for application in the current invention.

In a preferred embodiment the recombinant yeast cell is therefore a recombinant yeast cell further functionally expressing:
a nucleic acid sequence encoding a protein comprising phosphoketolase activity (EC 4.1.2.9 or EC 4.1.2.22, PKL); and/or
a nucleic acid sequence encoding a protein having phosphotransacetylase (PTA) activity (EC 2.3.1.8); and/or
a nucleic acid sequence encoding a protein having acetate kinase (ACK) activity (EC 2.7.2.12). Preferences for the above proteins and the nucleic sequences encoding for such are as described in WO2014/081803, WO2015/148272 and WO2018172328A1, incorporated herein by reference.

WO2014/129898, WO2018/228836, WO 2018/114762 and WO2019/063542 describe a metabolic route including a protein having ribulose-1,5-biphosphate carboxylase oxygenase (Rubisco) activity, optionally one or more molecular chaperones for a protein having ribulose-1,5-biphosphate carboxylase oxygenase (Rubisco) activity, and a protein having phosphoribulokinase (PRK) activity and recombinant yeast cells comprising such a metabolic route. The metabolic routes described in WO2014/129898, WO2018/228836, WO 2018/114762 and WO2019/063542 are preferred redox sinks and incorporated herein by reference. The genetic modifications and embodiments described for the cell in the claims of WO2014/129898, WO2018/228836, WO 2018/114762 and WO2019/063542, incorporated herein by reference, can advantageously also be present in the recombinant yeast cell of the invention.

In a preferred embodiment the recombinant yeast cell is therefore a recombinant yeast cell further functionally expressing:
a nucleic acid sequence encoding a protein having ribulose-1,5-biphosphate carboxylase oxygenase (Rubisco) activity; and/or
a nucleic acid sequence encoding a protein having phosphoribulokinase (PRK) activity; and/or
optionally a nucleic acid sequence encoding one or more molecular chaperones for the protein having ribulose-1,5-biphosphate carboxylase oxygenase (Rubisco) activity. Preferences for the above proteins and the nucleic sequences encoding for such are as described in WO2014/129898, WO2018/228836 and WO2019/063542.

WO2015/028582 describes examples of a protein comprising NADH dependent acetylating acetaldehyde dehydrogenase activity and metabolic routes incorporating such. The genetic modifications and embodiments described for the cell in the claims of WO2015028582, incorporated herein by reference, can advantageously also be present as a redox sink in the recombinant yeast cell of the invention.

In a preferred embodiment the recombinant yeast cell is therefore a recombinant yeast cell further functionally expressing:
a, preferably heterologous, nucleic acid sequence encoding a protein comprising NADH dependent acetylating acetaldehyde dehydrogenase activity; and/or
a, preferably heterologous, nucleic acid sequence encoding a protein comprising acetyl-CoA synthetase activity; and/or
a, preferably heterologous, nucleic acid sequence encoding a protein comprising alcohol dehydrogenase activity.

Preferences for the above proteins and the nucleic sequences encoding for such are as described in WO2015/028582.

### PPP-Genes

The recombinant yeast cell in the invention may further comprise one or more genetic modifications that increases the flux of the pentose phosphate pathway. The genes encoding for this pentose phosphate pathway are herein also referred to as the “PPP” genes.

In a preferred host cell, the genetic modification comprises overexpression of at least one enzyme of the (non-oxidative part) pentose phosphate pathway. Preferably the enzyme is selected from the group consisting of the enzymes encoding for ribulose-5-phosphate isomerase, ribulose-5-phosphate epimerase, transketolase and transaldolase. Various combinations of enzymes of the (non-oxidative part) pentose phosphate pathway may be overexpressed. E.g. the enzymes that are overexpressed may be at least the enzymes ribulose-5-phosphate isomerase and ribulose-5-phosphate epimerase; or at least the enzymes ribulose-5-phosphate isomerase and transketolase; or at least the enzymes ribulose-5-phosphate isomerase and transaldolase; or at least the enzymes ribulose-5-phosphate epimerase and transketolase; or at least the enzymes ribulose-5-phosphate epimerase and transaldolase; or at least the enzymes transketolase and transaldolase; or at least the enzymes ribulose-5-phosphate epimerase, transketolase and transaldolase; or at least the enzymes ribulose-5-phosphate isomerase, transketolase and transaldolase; or at least the enzymes ribulose-5-phosphate isomerase, ribulose-5-phosphate epimerase, and transaldolase; or at least the enzymes ribulose-5-phosphate isomerase, ribulose-5-phosphate epimerase, and transketolase.

Possibly each of the enzymes ribulose-5-phosphate isomerase, ribulose-5-phosphate epimerase, transketolase and transaldolase are overexpressed in the host cell. More preferred is a host cell in which the genetic modification comprises at least overexpression of both the enzymes transketolase and transaldolase.

The enzyme “ribulose 5-phosphate epimerase” (EC 5.1.3.1) is herein defined as an enzyme that catalyses the epimerisation of D-xylulose 5-phosphate into D-ribulose 5-phosphate and vice versa. The enzyme is also known as phosphoribulose epimerase; erythrose-4-phosphate isomerase; phosphoketopentose 3-epimerase; xylulose phosphate 3-epimerase; phosphoketopentose epimerase; ribulose 5-phosphate 3-epimerase; D-ribulose phosphate-3-epimerase; D-ribulose 5-phosphate epimerase; D-ribulose-5-P 3-epimerase; D-xylulose-5-phosphate 3-epimerase; pentose-5-phosphate 3-epimerase; or D-ribulose-5-phosphate 3-epimerase. A ribulose 5-phosphate epimerase may be further defined by its amino acid sequence. Likewise a ribulose 5-phosphate epimerase may be defined by a nucleotide sequence encoding the enzyme as well as by a nucleotide sequence hybridising to a reference nucleotide sequence encoding a ribulose 5-phosphate epimerase. The nucleotide sequence encoding for ribulose 5-phosphate epimerase is herein designated RPE1.

The enzyme “ribulose 5-phosphate isomerase” (EC 5.3.1.6) is herein defined as an enzyme that catalyses direct isomerisation of D-ribose 5-phosphate into D-ribulose 5-phosphate and vice versa. The enzyme is also known as phosphopentosisomerase; phosphoriboisomerase; ribose phosphate isomerase; 5-phosphoribose isomerase; D-ribose 5-phosphate isomerase; D-ribose-5-phosphate ketol-isomerase; or D-ribose-5-phosphate aldose-ketose-isomerase. A ribulose 5-phosphate isomerase may be further defined by its amino acid sequence. Likewise a ribulose 5-phosphate isomerase may be defined by a nucleotide sequence encoding the enzyme as well as by a nucleotide sequence hybridising to a reference nucleotide sequence encoding a ribulose 5-phosphate isomerase. The nucleotide sequence encoding for ribulose 5-phosphate isomerase is herein designated RKI1.

The enzyme “transketolase” (EC 2.2.1.1) is herein defined as an enzyme that catalyses the reaction: D-ribose 5-phosphate+D-xylulose 5-phosphate<->sedoheptulose 7-phosphate+D-glyceraldehyde 3-phosphate and vice versa. The enzyme is also known as glycolaldehydetransferase or sedoheptulose-7-phosphate: D-glyceraldehyde-3-phosphate glycolaldehydetransferase. A transketolase may be further defined by its amino acid. Likewise a transketolase may be defined by a nucleotide sequence encoding the enzyme as well as by a nucleotide sequence hybridising to a reference nucleotide sequence encoding a transketolase. The nucleotide sequence encoding for transketolase is herein designated TKL1.

The enzyme “transaldolase” (EC 2.2.1.2) is herein defined as an enzyme that catalyses the reaction: sedoheptulose 7-phosphate+D-glyceraldehyde 3-phosphate<->D-erythrose 4-phosphate+D-fructose 6-phosphate and vice versa. The enzyme is also known as dihydroxyacetonetransferase; dihydroxyacetone synthase; formaldehyde transketolase; or sedoheptulose-7-phosphate: D-glyceraldehyde-3-phosphate glyceronetransferase. A transaldolase may be further defined by its amino acid sequence. Likewise a transaldolase may be defined by a nucleotide sequence encoding the enzyme as well as by a nucleotide sequence hybridising to a reference nucleotide sequence encoding a transaldolase. The nucleotide sequence encoding for transketolase from is herein designated TAL1.

### Deletion or Disruption of Glycerol 3-Phosphate Phosphohydrolase and/or Glycerol 3-Phosphate Dehydrogenase

The recombinant yeast cell further may or may not comprise a deletion or disruption of one or more endogenous nucleotide sequence encoding a glycerol 3-phosphate phosphohydrolase gene and/or encoding a glycerol 3-phosphate dehydrogenase gene.

Preferably enzymatic activity needed for the NADH-dependent glycerol synthesis in the yeast cell is reduced or deleted. The reduction or deletion of the enzymatic activity of glycerol 3-phosphate phosphohydrolase and/or glycerol 3-phosphate dehydrogenase can be achieved by modifying one or more genes encoding a NAD-dependent glycerol 3-phosphate dehydrogenase (GPD) and/or one or more genes encoding a glycerol phosphate phosphatase (GPP), such that the enzyme is expressed considerably less than in the wild-type or such that the gene encodes a polypeptide with reduced activity. Such modifications can be carried out using commonly known biotechnological techniques, and may in particular include one or more knock-out mutations or site-directed mutagenesis of promoter regions or coding regions of the structural genes encoding GPD and/or GPP. Alternatively, yeast strains that are defective in glycerol production may be obtained by random mutagenesis followed by selection of strains with reduced or absent activity of GPD and/or GPP. S. cerevisiae GPD1, GPD2, GPP1 and GPP2 genes are shown in WO2011010923, and are disclosed in SEQ ID NO: 24-27 of that application.

Preferably the recombinant yeast is a recombinant yeast that further comprises a deletion or disruption of a glycerol-3-phosphate dehydrogenase (GPD) gene. The one or more of the glycerol phosphate phosphatase (GPP) genes may or may not be deleted or disrupted.

More preferably the recombinant yeast is a recombinant yeast that comprises a deletion or disruption of a glycerol-3-phosphate dehydrogenase 1 (GPD1) gene. The glycerol-3-phosphate dehydrogenase 2 (GPD2) gene may or may not be deleted or disrupted.

Most preferably the recombinant yeast is a recombinant yeast that comprises a deletion or disruption of a glycerol-3-phosphate dehydrogenase 1 (GPD1) gene, whilst the glycerol-3-phosphate dehydrogenase 2 (GPD2) gene remains active and/or intact. Preferably therefore, only one of the S. cerevisiae GPD1, GPD2, GPP1 and GPP2 genes is disrupted and deleted, whereas most preferably only GPD1 is chosen from the group consisting of GPD1, GPD2, GPP1 and GPP2 genes to be disrupted or deleted.

Without wishing to be bound to any kind of theory it is believed that a recombinant yeast according to the invention wherein the GPD1 gene, but not the GPD2 gene, is deleted or disrupted, can be advantageous when applied in a fermentation process where the glucose at the start of or during the fermentation, is preferably equal to or more than 80 g/L, more preferably equal to or more than 90 g/L, even more preferably equal to or more than 100 g/L, still more preferably equal to or more than 110 g/L, yet even more preferably equal to or more than 120 g/L, equal to or more than 130 g/L, equal to or more than 140 g/L, equal to or more than 150 g/L, equal to or more than 160 g/L, equal to or more than 170 g/L, or equal to or more than 180 g/L.

Preferably at least one gene encoding a GPD and/or at least one gene encoding a GPP is entirely deleted, or at least a part of the gene is deleted that encodes a part of the enzyme that is essential for its activity. Good results can be achieved with a S. cerevisiae cell, wherein the open reading frames of the GPD1 gene and/or of the GPD2 gene have been inactivated. Inactivation of a structural gene (target gene) can be accomplished by a person skilled in the art by synthetically synthesizing or otherwise constructing a DNA fragment consisting of a selectable marker gene flanked by DNA sequences that are identical to sequences that flank the region of the host cell's genome that is to be deleted. Suitably, good results can be been obtained with the inactivation of the GPD1 and GPD2 genes in Saccharomyces cerevisiae by integration of the marker genes kanMX and hphMX4. Subsequently this DNA fragment is transformed into a host cell. Transformed cells that express the dominant marker gene are checked for correct replacement of the region that was designed to be deleted, for example by a diagnostic polymerase chain reaction or Southern hybridization.

Thus, in the recombinant yeast cells of the invention, glycerol 3-phosphate phosphohydrolase activity in the cell and/or glycerol 3-phosphate dehydrogenase activity in the cell can be advantageously reduced.

### Glycerol Re-Uptake

The recombinant yeast cell may or may not further comprise one or more additional nucleic acid sequences that are part of a glycerol re-uptake pathway. That is, the recombinant yeast cell may or may not functionally express:
a nucleic acid sequence encoding for a protein having glycerol dehydrogenase activity (E.C. 1.1.1.6);
a nucleic acid sequence encoding a protein having dihydroxyacetone kinase activity (E.C. 2.7.1.28 or E.C. 2.7.1.29); and
optionally a nucleic acid sequence encoding a protein having glycerol transporter activity.

Without wishing to be bound by any kind of theory it is believed that a recombinant yeast cell that further comprises a combination of glycerol dehydrogenase, dihydroxyacetone kinase and optionally a glycerol transporter has an improved overall performance in the form of higher ethanol yields.

Preferences for the above proteins and the nucleic sequences encoding for such are as described in WO2015/028582 and WO 2018/114762, incorporated herein by reference.

A recombinant yeast cell as described in WO 2018/114762, further incorporating the nucleotide sequences for the glucosidases as described herein is especially preferred.

### Recombinant Expression

The recombinant yeast cell is a recombinant cell. That is to say, a recombinant yeast cell comprises, or is transformed with or is genetically modified with a nucleotide sequence that does not naturally occur in the cell in question. Techniques for the recombinant expression of enzymes in a cell, as well as for the additional genetic modifications of a recombinant yeast cell are well known to those skilled in the art. Typically such techniques involve transformation of a cell with nucleic acid construct comprising the relevant sequence. Such methods are, for example, known from standard handbooks, such as Sambrook and Russel (2001) “Molecular Cloning: A Laboratory Manual”, (3rd edition), published by Cold Spring Harbor Laboratory Press, or F. Ausubel et al., eds., “Current protocols in molecular biology”, Green Publishing and Wiley Interscience, New York (1987). Methods for transformation and genetic modification of fungal host cells are known from e.g. EP-A-0635574, WO98/46772, WO 99/60102, WO00/37671, WO90/14423, EP-A-0481008, EP-A-0635574 and U.S. Pat. No. 6,265,186.

### Fermentation Process

The invention provides a process for the production of ethanol, comprising fermentation of a feed, preferably a carbon source, preferably a carbohydrate or another organic carbon source, using a recombinant yeast cell as described in this specification, thereby forming ethanol.

The feed for this fermentation process may comprise one or more fermentable carbon sources. The fermentable carbon source preferably comprises or is consisting of one or more fermentable carbohydrates.

The feed suitably contains at least one di-saccharide, oligo-saccharide and/or poly-saccharide. Preferably the feed contains a mixture of mono-saccharide(s), di-saccharide(s), oligo-saccharide(s) and/or poly-saccharide(s). For example, the fermentable carbon source may comprise one or more carbohydrates selected from the group consisting of glucose, fructose, sucrose, maltose, isomaltose, maltotriose, panose, xylose, arabinose, galactose, mannose and trehalose. The feed, preferably comprising or consisting of one or more carbohydrates, may suitably be obtained or derived from starch, cellulose, hemicellulose lignocellulose, and/or pectin. Preferably the feed is obtained, derived or comprises amylase and or amylopectin. Suitably the feed, preferably in the form of a fermentable carbon source, may be in the form of a, preferably aqueous, slurry, suspension, or a liquid.

The concentration of fermentable carbohydrate, such as for example glucose, during fermentation is preferably equal to or more than 80 g/L. That is, the initial concentration of glucose at the start of the fermentation, is preferably equal to or more than 80 g/L, more preferably equal to or more than 90 g/L, even more preferably equal to or more than 100 g/L, still more preferably equal to or more than 110 g/L, yet even more preferably equal to or more than 120 g/L, equal to or more than 130 g/L, equal to or more than 140 g/L, equal to or more than 150 g/L, equal to or more than 160 g/L, equal to or more than 170 g/L, or equal to or more than 180 g/L. The start of the fermentation may be the moment when the fermentable fermentable carbohydrate is brought into contact with the recombinant cell of the invention.

The fermentable carbon source may be prepared by contacting starch, lignocellulose, and/or pectin with an enzyme composition, wherein one or more mono-saccharides, disaccharides and/or polysaccharides are produced, and wherein the produced mono-saccharides, disaccharides and/or polysaccharides are subsequently fermented to give a fermentation product.

Before enzymatic treatment, the lignocellulosic material may be pretreated. The pretreatment may comprise exposing the lignocellulosic material to an acid, a base, a solvent, heat, a peroxide, ozone, mechanical shredding, grinding, milling or rapid depressurization, or a combination of any two or more thereof. This chemical pretreatment is often combined with heat-pretreatment, e.g. between 150-220° C. for 1 to 30 minutes. Subsequently the pretreated material can be subjected to enzymatic hydrolysis to release sugars that may be fermented according to the invention. This may be executed with conventional methods, e.g. contacting with cellulases, for instance cellobiohydrolase(s), endoglucanase(s), beta-glucosidase(s) and optionally other enzymes, The conversion with the cellulases may be executed at ambient temperatures or at higher temperatures, at a reaction time to release sufficient amounts of sugar(s). The result of the enzymatic hydrolysis is hydrolysis product comprising C5/C6 sugars, herein designated as the sugar composition.

In one embodiment the fermentable carbohydrate is, or is comprised by a biomass hydrolysate, such as a corn stover or corn fiber hydrolysate. Such biomass hydrolysate may in its turn comprise, or be derived from corn stover and/or corn fiber.

By a “hydrolysate” is herein understood a polysaccharide-comprising material (such as corn stover, corn starch, corn fiber, or lignocellulosic material, which polysaccharides have been depolymerized through the addition of water to form mono and oligosaccharide sugars. Hydrolysates may be produced by enzymatic or acid hydrolysis of the polysaccharide-containing material.

A biomass hydrolysate may be a lignocellulosic biomass hydrolysate. Lignocellulose herein includes hemicellulose and hemicellulose parts of biomass. Also lignocellulose includes lignocellulosic fractions of biomass. Suitable lignocellulosic materials may be found in the following list: orchard primings, chaparral, mill waste, urban wood waste, municipal waste, logging waste, forest thinnings, short-rotation woody crops, industrial waste, wheat straw, oat straw, rice straw, barley straw, rye straw, flax straw, soy hulls, rice hulls, rice straw, corn gluten feed, oat hulls, sugar cane, corn stover, corn stalks, corn cobs, corn husks, switch grass, miscanthus, sweet sorghum, canola stems, soybean stems, prairie grass, gamagrass, foxtail; sugar beet pulp, citrus fruit pulp, seed hulls, cellulosic animal wastes, lawn clippings, cotton, seaweed, algae (including macroalgae and microalgae), trees, softwood, hardwood, poplar, pine, shrubs, grasses, wheat, wheat straw, sugar cane bagasse, corn, corn husks, corn hobs, corn kernel, fiber from kernels, products and by-products from wet or dry milling of grains, municipal solid waste, waste paper, yard waste, herbaceous material, agricultural residues, forestry residues, municipal solid waste, waste paper, pulp, paper mill residues, branches, bushes, canes, corn, corn husks, an energy crop, forest, a fruit, a flower, a grain, a grass, a herbaceous crop, a leaf, bark, a needle, a log, a root, a sapling, a shrub, switch grass, a tree, a vegetable, fruit peel, a vine, sugar beet pulp, wheat midlings, oat hulls, hard or soft wood, organic waste material generated from an agricultural process, forestry wood waste, or a combination of any two or more thereof. Algae, such as macroalgae and microalgae have the advantage that they may comprise considerable amounts of sugar alcohols such as sorbitol and/or mannitol. Lignocellulose, which may be considered as a potential renewable feedstock, generally comprises the polysaccharides cellulose (glucans) and hemicelluloses (xylans, heteroxylans and xyloglucans). In addition, some hemicellulose may be present as glucomannans, for example in wood-derived feedstocks. The enzymatic hydrolysis of these polysaccharides to soluble sugars, including both monomers and multimers, for example glucose, cellobiose, xylose, arabinose, galactose, fructose, mannose, rhamnose, ribose, galacturonic acid, glucuronic acid and other hexoses and pentoses occurs under the action of different enzymes acting in concert. In addition, pectins and other pectic substances such as arabinans may make up considerably proportion of the dry mass of typically cell walls from non-woody plant tissues (about a quarter to half of dry mass may be pectins). Lignocellulosic material may be pretreated. The pretreatment may comprise exposing the lignocellulosic material to an acid, a base, a solvent, heat, a peroxide, ozone, mechanical shredding, grinding, milling or rapid depressurization, or a combination of any two or more thereof. This chemical pretreatment is often combined with heat-pretreatment, e.g. between 150-220° C. for 1 to 30 minutes.

The process for the production of ethanol may comprise an aerobic propagation step and an anaerobic fermentation step. More preferably the process according to the invention is a process comprising an aerobic propagation step wherein the population of the recombinant yeast cell is increased; and an anaerobic fermentation step wherein the carbon source is converted to ethanol by using the recombinant yeast cell population.

By propagation is herein understood a process of recombinant yeast cell growth that leads to increase of an initial recombinant yeast cell population. Main purpose of propagation is to increase the population of the recombinant yeast cell using the recombinant yeast cell's natural reproduction capabilities as living organisms. That is, propagation is directed to the production of biomass and is not directed to the production of ethanol. The conditions of propagation may include adequate carbon source, aeration, temperature and nutrient additions. Propagation is an aerobic process, thus the propagation tank must be properly aerated to maintain a certain level of dissolved oxygen. Adequate aeration is commonly achieved by air inductors installed on the piping going into the propagation tank that pull air into the propagation mix as the tank fills and during recirculation. The capacity for the propagation mix to retain dissolved oxygen is a function of the amount of air added and the consistency of the mix, which is why water is often added at a ratio of between 50:50 to 90:10 mash to water. “Thick” propagation mixes (80:20 mash-to-water ratio and higher) often require the addition of compressed air to make up for the lowered capacity for retaining dissolved oxygen. The amount of dissolved oxygen in the propagation mix is also a function of bubble size, so some ethanol plants add air through spargers that produce smaller bubbles compared to air inductors. Along with lower glucose, adequate aeration is important to promote aerobic respiration during propagation, making the environment during propagation different from the anaerobic environment during fermentation.

By an anaerobic fermentation process is herein understood a fermentation step run under anaerobic conditions.

The anaerobic fermentation is preferably run at a temperature that is optimal for the cell. Thus, for most recombinant yeast cells, the fermentation process is performed at a temperature which is less than about 50° C., less than about 42° C., or less than about 38° C. For recombinant yeast cell or filamentous fungal host cells, the fermentation process is preferably performed at a temperature which is lower than about 35, about 33, about 30 or about 28° C. and at a temperature which is higher than about 20, about 22, or about 25° C.

The ethanol yield, based on xylose and/or glucose, in the process according to the invention is preferably at least about 50, about 60, about 70, about 80, about 90, about 95 or about 98%. The ethanol yield is herein defined as a percentage of the theoretical maximum yield.

The process according to the invention, and the propagation step and/or fermentation step suitably comprised therein can be carried out in batch, fed-batch or continuous mode. A separate hydrolysis and fermentation (SHF) process or a simultaneous saccharification and fermentation (SSF) process may also be applied.

The process according to the invention can therefore advantageously be a process, wherein the process comprises an enzymatic hydrolysis step and a fermentation step, wherein both steps are carried out simultaneously, preferably in the same vessel.

Alternatively, the process according to the invention can advantageously be a process comprises an enzymatic hydrolysis step and a fermentation step, wherein the enzymatic hydrolysis step is carried out separately from the fermentation step, preferably in a separate vessel, and is preferably preceding the fermentation step.

### Further Preferences for the Feed

The recombinant yeast and process according to the invention advantageously allow for less residual sugar at the end of fermentation and/or a higher ethanol yield more robust process.

Advantageously the process, or any anaerobic fermentation during the process can therefore be carried out in the presence of high concentrations of disaccharides, oligosaccharides and/or polysaccharides. By an oligosaccharide is herein preferably understood a saccharide comprising 3 to 30 saccharide units, more preferably 3 to 10 saccharide units and most preferably 3 to 5 saccharide units.

Preferably the process according to the invention is a process, wherein the total weight percentage of di-saccharide, oligo-saccharide and poly-saccharide, based on the total weight of saccharides present in the feed, is equal to or more than 1% w/w, preferably equal to or more than 5% w/w, more preferably equal to or more than 10% w/w and most preferably equal to or more than 20% w/w. Most preferably the wherein the total weight percentage of di-saccharide, oligo-saccharide and poly-saccharide, based on the total weight of saccharides present in the feed, lies in the range from equal to or more than 1% w/w to equal to or less than 100% w/w, more preferably in the range from equal to or more than 2% w/w to equal to or less than 60% w/w, and most preferably in the range from equal to or more than 5% w/w to equal to or less than 50% w/w.

More preferably the total weight percentage of disaccharides and/or oligosaccharides, based on the weight of saccharides present in the feed, is equal to or more than 1% w/w, equal to or more than 2% w/w, equal to or more than 3% w/w, equal to or more than 5% w/w, equal to or more than 10% w/w or equal to or more than 20% w/w. Most preferably the total weight percentage of disaccharides and/or oligosaccharides, based on the weight of saccharides present in the feed, lies in the range from equal to or more than 1% w/w to equal to or less than 100% w/w, more preferably in the range from equal to or more than 2% w/w to equal to or less than 60% w/w, and most preferably in the range from equal to or more than 5% w/w to equal to or less than 50% w/w.

More preferably the disaccharides and/or oligo-saccharides are chosen from the group consisting of maltose, isomaltose, maltotriose, panose, trehalose, cellobiose, pullulan, cellobiose, sophorose, laminaribiose, gentibiose and combinations thereof.

More preferably the feed in the process according to the invention comprises one or more compounds comprising an alpha-1,6-glycosidic bond and/or a beta-1,2-glycosidic bond, beta-1,3-glycosidic bond, beta-1,4-glycosidic bond, beta-1,6-glycosidic bond and/or an alpha-1,1-glycosidic bond.

Preferably the process according to the invention therefore comprises fermentation of a feed, wherein the feed contains
a first di-saccharide, oligo-saccharide and/or poly-saccharide consisting of two or more mono-saccharide units linked to each other via an alpha-1,4-glycosidic bond;
and
a further di-saccharide, oligo-saccharide and/or poly-saccharide containing at least two mono-saccharide units linked to each other via an alpha-1,6-glycosidic bond, an alpha-1,1-glycosidic bond or a beta-1,4-glycosidic bond.

More preferably the process according to the invention comprises fermentation of a feed, wherein the feed contains
a di-saccharide, oligo-saccharide and/or poly-saccharide consisting of two or more mono-saccharide units linked to each other via an alpha-1,4-glycosidic bond; and/or
a di-saccharide, oligo-saccharide and/or poly-saccharide containing at least two mono-saccharide units linked to each other via an alpha-1,6-glycosidic bond; and/or
a di-saccharide, oligo-saccharide and/or poly-saccharide containing at least two mono-saccharide units linked to each other via a beta-glycosidic bond, preferably via a beta-1,2-glycosidic bond, beta-1,3-glycosidic bond, beta-1,4-glycosidic bond and/or beta-1,6-glycosidic bond; and/or
a di-saccharide, oligo-saccharide and/or poly-saccharide containing at least two mono-saccharide units linked to each other via an alpha-1,1-glycosidic bond.

The process, respectively any anaerobic fermentation therein, is therefore preferably carried out with a feed comprising
a maltose concentration of 1 g/L or more, 2 g/L or more, 3 g/L or more, 4 g/L or more, 5 g/L or more, 10 g/L or more, 15 g/L or more, 20 g/L or more, 25 g/L or more, 30 g/L or more, 40 g/L or more, 50 g/L or more, 75 g/L or more, or 100 g/L or more or may for example be in the range of 1 g/L-200 g/L, 1 g/L-100 g/L, or 3 g/L-50 g/L; and/or
an isomaltose concentration of 1 g/L or more, 2 g/L or more, 3 g/L or more, 4 g/L or more, 5 g/L or more, 10 g/L or more, 15 g/L or more, 20 g/L or more, 25 g/L or more, 30 g/L or more, 40 g/L or more, 50 g/L or more, 75 g/L or more, or 100 g/L or more or may for example be in the range of 1 g/L-200 g/L, 1 g/L-100 g/L, or 3 g/L-50 g/L; and/or
a maltotriose concentration of 1 g/L or more, 2 g/L or more, 3 g/L or more, 4 g/L or more, 5 g/L or more, 10 g/L or more, 15 g/L or more, 20 g/L or more, 25 g/L or more, 30 g/L or more, 40 g/L or more, 50 g/L or more, 75 g/L or more, or 100 g/L or more or may for example be in the range of 1 g/L-200 g/L, 1 g/L-100 g/L, or 3 g/L-50 g/L; and/or
a panose concentration of 1 g/L or more, 2 g/L or more, 3 g/L or more, 4 g/L or more, 5 g/L or more, 10 g/L or more, 15 g/L or more, 20 g/L or more, 25 g/L or more, 30 g/L or more, 40 g/L or more, 50 g/L or more, 75 g/L or more, or 100 g/L or more or may for example be in the range of 1 g/L-200 g/L, 1 g/L-100 g/L, or 3 g/L-50 g/L; and/or
a DP4+ concentration (i.e. the total amount or concentration of oligosaccharides comprising 4 or more monosaccharide (for example glucose) units) of 1 g/L or more, 2 g/L or more, 3 g/L or more, 4 g/L or more, 5 g/L or more, 10 g/L or more, 15 g/L or more, 20 g/L or more, 25 g/L or more, 30 g/L or more, 40 g/L or more, 50 g/L or more, 75 g/L or more, 100 g/L or more, 200 g/L or more, 300 g/L or more, 400 g/L or more, or 500 g/L or more or may for example be in the range of 1 g/L-1000 g/L, 1 g/L-500 g/L, or 3 g/L-200 g/L.

For the recovery of the fermentation product existing technologies are used. For different fermentation products different recovery processes are appropriate. Existing methods of recovering ethanol from aqueous mixtures commonly use fractionation and adsorption techniques. For example, a beer still can be used to process a fermented product, which contains ethanol in an aqueous mixture, to produce an enriched ethanol-containing mixture that is then subjected to fractionation (e.g., fractional distillation or other like techniques). Next, the fractions containing the highest concentrations of ethanol can be passed through an adsorber to remove most, if not all, of the remaining water from the ethanol. In an embodiment in addition to the recovery of fermentation product, the yeast may be recycled.

All patent and literature references cited in the present specification are hereby incorporated by reference in their entirety.

### EXAMPLES

The following examples are offered for illustrative purposes only, and are not intended to limit the scope of the present invention in any way.

### General Molecular Biology Techniques

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

### Starter Strain

Strains were prepared using Ethanol Red® as starting strain. Ethanol Red® is a commercial Saccharomyces cerevisiae strain, available from Lesaffre.

A strain construction approach that can be followed is described in WO2013/144257A1 and WO2015/028582, incorporated herein by reference.

Expression cassettes from various genes of interest can be recombined in vivo into a pathway at a specific locus upon transformation of this yeast (U.S. Pat. No. 9,738,890 B2). The promoter, ORF and terminator sequences are assembled into expression cassettes with Golden Gate technology, as described by Engler et al (2011) and ligated into BsaI-digested backbone vectors that decorated the expression cassettes with the connectors for the in vivo recombination step. The expression cassettes including connectors are amplified by PCR. In addition, a 5′- and a 3′-DNA fragment of the up- and downstream part of the integration locus was amplified using PCR and decorated by a connector sequence. Upon transformation of yeast cells with these DNA fragments, in vivo recombination and integration into the genome takes place at the desired location. CRISPR-Cas9 technology is used to make a unique double stranded break at the integration locus to target the pathway to this specific locus (DiCarlo et al., 2013, Nucleic Acids Res 41:4336-4343) and WO16110512 and US2019309268. The gRNA was expressed from a multi-copy yeast shuttling vector that contains a natMX marker which confers resistance to the yeast cells against the antibiotic substance nourseothricin (NTC). The backbone of this plasmid is based on pRS305 (Sikorski and Hieter, Genetics 1989, vol. 122, pp. 19-27), including a functional 2 micron ORI sequence. The Streptococcus pyogenes CRISPR-associated protein 9 (Cas9) was expressed from a pRS414 plasmid (Sikorski and Hieter, 1989) with kanMX marker which confers resistance to the yeast cells against the antibiotic substance geneticin (G418). The guide RNA and protospacer sequences were designed with a gRNA designer tool (see for example https://www.atum.bio/eCommerce/cas9/input).

In the examples below new enzyme expressing strains were constructed by transforming the S. cerevisiae host cell with enzyme expression cassettes as described below. Synthetic DNA sequences were ordered at TWIST (South San Francisco, CA 94080, USA). or Thermofisher-GeneArt (Regensburg, Germany).

An overview of the strains used in these examples is provided in Table 2 below. An overview of the promoters and terminators used in these examples is provided in Table 3 below.

TABLE 2
S. cerevisiae strains used in the examples

| Strain name | Genotype |
|---|---|
| Parental strain | Wildtype (Ethanol Red ®) |
| intermediate strain IX1 | Δhis3 |
| | cbbM HIS3 - GroEL- prk -GroES |
| | Sc_DAK1 - Ec_gldA -Zrou_T5 |
| Comparative strain A, expressing | IX1, INT59::PGK1.pro-GA.orf_0048-ENO1.ter |
| P. strigosozonata alpha 1,4-glucosidase | |
| Example strain NX1 expressing | IX1, INT59::2J- |
| P. strigosozonata alpha 1,4-glucosidase; | Spar_TDH3.pro-Pstr_GA.orf_0009-Sc_ADH1.ter-2K- |
| T. coccinea alpha 1,6-glucosidase; | Sc_PFY1.pro-Tcoc_GLA.orf-Sc_TDH1.ter-2L- |
| A. kawachii beta-glucosidase; | Sc_ACT1.pro-Akaw_BG17.orf-Sc_ENO1.ter-2M- |
| T. cellulolyticus alpha 1,1-glucosidase; | Sc_YKT6.pro-Tcel_Tre17.orf-Sc_CYC1.ter-2N- |
| Example strain NX2 expressing | IX1, INT59::2J- |
| P. strigosozonata alpha 1,4-glucosidase; | Sc_PGK1.pro-Pstr_GA.orf_0009-Sc_ENO1.ter-2K |
| T. coccinea alpha 1,6-glucosidase; | Sc_RPS3.pro-Tcoc_GLA.orf-Sc_TDH1.ter-2L- |
| A. kawachii beta-glucosidase; | Sc_ACT1.pro-Akaw_BG17.orf-Sc_ENO1.ter-2M |
| T. cellulolyticus alpha 1,1-glucosidase; | Sc_YKT6.pro-Tcel_Tre17.orf-Sc_CYC1.ter-2N |
| Example strain NX3 expressing | IX1, INT59::2J- |
| P. strigosozonata alpha 1,4-glucosidase; | Sc_PGK1.pro-Pstr_GA.orf_0009-Sc_ENO1.ter-2K- |
| T. coccinea alpha 1,6-glucosidase; | Sc_RPS3.pro-Tcoc_GLA.orf-Sc_TDH1.ter-2L- |
| A. kawachii beta-glucosidase; | Sc_ZUO1.pro-Akaw_BG17.orf-Sc_ADH1.ter-2M- |
| T. cellulolyticus alpha 1,1-glucosidase; | Sc_MYO4.pro-Tcel_Tre17.orf-Sc_AQR1.ter-2N- |

### Intermediate “Rubisco” strain (IX1)

The starter strain was transformed with the cbbM gene encoding the single subunit of ribulose-1,5-biphosphate-carboxylase (RuBisCO) from Thiobacfflus denitrificans and the genes encoding chaperonins GroEL and GroES from E. coli to aid in the proper folding of the RuBisCO protein in the cytosol of S. cerevisiae in a similar manner as described in WO 2018/114762. In addition a nucleotide sequence encoding phosphoribulokinase (prk) was incorporated in a similar manner as described in WO 2018/114762. In a next step, nucleotide sequences encoding NAD+ linked glycerol dehydrogenase (EC 1.1.1.6), dihydroxyacetone kinase and Z. rouxii T5 glycerol transporter were incorporated in a similar manner as described in WO 2018/114762.

The above resulted in intermediate strain IX1 with a genotype as illustrated in Table 2.

### Comparative Example A: Construction of Comparative Strain A

### (Yeast Strain Expressing Alpha 1,4-Glucosidase (Glucoamylase, EC 3.2.1.3))

Comparative strain A was constructed by transforming the intermediate strain IX1 mentioned above with an expression cassette comprising the S. cerevisiae PGK1 promoter (see SEQ ID NO: xx), a gene encoding glucoamylase from Punctularia strigosozonata (see SEQ ID NO: 1 and SEQ ID NO: 2, Pstr_GA.orf_0048) as the gene of interest and the S. cerevisiae ENO1 terminator (see SEQ ID NO: xx).

### Example 1: Construction of Example Strain NX1

### (Yeast Strain Expressing Alpha 1,4-Glucosidase (Glucoamylase, EC 3.2.1.3), Alpha 1,6-Glucosidase (Debranching Glucoamylase, EC 3.2.1.10), Beta-Glucosidase (EC 3.2.1.21) and Alpha 1,4-Glucosidase (Trehalase, EC 3.2.1.28))

Example strain NX1 was constructed by transforming the intermediate strain IX1 mentioned above with four expression cassettes:
Expression cassette “fragment A”: 2J-Spar_TDH3.pro-Pstr_GA.orf_0009-Sc_ADH1.ter-2K
Expression cassette “fragment B”: 2K-Sc_PFY1.pro-Tcoc_GLA.orf-Sc_TDH1.ter-2L
Expression cassette “fragment C”: 2 L-Sc_ACT1.pro-Akaw_BG17.orf-Sc_ENO1.ter-2M
Expression cassette “fragment D”: 2M-Sc_YKT6.pro-Tcel_Tre17.orf-Sc_CYC1.ter-2N

Expression cassette “fragment A”: The first cassette named “fragment A” was compiled using Golden Gate Cloning and comprised the Saccharomyces paradoxus TDH3 promoter (Spar_TDH3.pro), the Pstr_GA.orf_0009 orf and S. cerevisiae ADH11 terminator (Sc_ ADH1.ter). The cassette was decorated with 50 bp connectors 2J and 2K, as illustrated in: SEQ ID NO: 38 and SEQ ID NO: 39, respectively. The nucleic acid sequence of the DNA fragment A s illustrated in SEQ ID NO: 43.

Expression cassette “fragment B”: The second cassette named “fragment B” comprised S. cerevisiae PYF1 promoter (Sc_PYF1.pro), Tcoc_GLA.orf and S. cerevisiae TDH1 terminator (Sc_TDH1.ter). The cassette was decorated with 50 bp connectors 2K and 2L, as illustrated in: SEQ ID NO: 39 and SEQ ID NO: 40, respectively. The nucleic acid sequence of the DNA fragment B is illustrated in SEQ ID NO: 44.

Expression cassette “fragment C”: The third cassette named “fragment C”, comprised the S. cerevisiae ACT1 promoter (Sc_ACT1.pro), Akaw_BG17.orf and S. cerevisiae ENO1 terminator (Sc_ENO1.ter). The cassette was decorated with 50 bp connectors 2L and 2M as illustrated in: SEQ ID NO: 40 and SEQ ID NO: 41, respectively. The nucleic acid sequence of the DNA fragment C is illustrated in SEQ ID NO: 45.

Expression cassette “fragment D”: The fourth cassette named “fragment D”, comprised the S. cerevisiae YKT6 promoter (Sc_YKT6.pro), Tcel_Tre17.orf and S. cerevisiae terminator (Sc_CYC1.ter). The cassette was decorated with 50 bp connectors 2M and 2N as illustrated in: SEQ ID NO: 41 and SEQ ID NO: 42, respectively. The nucleic acid sequence of the DNA fragment D is illustrated in SEQ ID NO: 46.

The above four cassettes were integrated in the intermediate strain in the INT59 locus on a non-coding region on chromosome XI, using CRISPR-Cas9 techniques as described above and the following sequences for homologous integration:
INT59_FLANK5 (illustrated by SEQ ID NO: 51); and
INT59_FLANK3 (illustrated by SEQ ID NO: 52)

Diagnostic PCR was performed to confirm the correct assembly and integration at the INT59 locus of the three expression cassettes. Plasmid free colonies were selected which resulted in example strain NX1 (see Table 2 for detailed genotypes).

### Example 2: Construction of Example Strain NX2

### (Yeast Strain Expressing Alpha 1,4-Glucosidase (Glucoamylase, EC 3.2.1.3), Alpha 1,6-Glucosidase (Debranching Glucoamylase, EC 3.2.1.10), Beta-Glucosidase (EC 3.2.1.21) and Alpha 1,4-Glucosidase (Trehalase, EC 3.2.1.28))

Example strain NX2 was constructed by transforming the intermediate strain IX1 mentioned above with four expression cassettes with four expression cassettes:
Expression cassette “fragment E”: 2J-Sc_PGK1.pro-Pstr_GA.orf_0009-Sc_ENO1.ter-2K
Expression cassette “fragment F”: 2K-Sc_RPS3.pro-Tcoc_GLA.orf-Sc_TDH1.ter-2L
Expression cassette “fragment C”: 2 L-Sc_ACT1.pro-Akaw_BG17.orf-Sc_ENO1.ter-2M
Expression cassette “fragment D”: 2M-Sc_YKT6.pro-Tcel_Tre17.orf-Sc_CYC1.ter-2N

Expression cassette “fragment E”: The first cassette named “fragment E” was compiled using Golden Gate Cloning and comprised the S. cerevisiae PGK1 promoter (Sc_ PGK1.pro), the Pstr_GA.orf_0009 orf and S. cerevisiae ENO1 terminator (Sc_ ENO1.ter). The cassette was decorated with 50 bp connectors 2J and 2K, as illustrated in: SEQ ID NO: 38 and SEQ ID NO: 39, respectively. The nucleic acid sequence of the DNA fragment E is illustrated in SEQ ID NO: 47.

Expression cassette “fragment F”: The second cassette named “fragment F” comprised S. cerevisiae RPS3 promoter (Sc_RPS3.pro), Tcoc_GLA.orf and S. cerevisiae TDH1 terminator (Sc_TDH1.ter). The cassette was decorated with 50 bp connectors 2K and 2L, as illustrated in: SEQ ID NO: 39 and SEQ ID NO: 40, respectively. The nucleic acid sequence of the DNA fragment is illustrated in SEQ ID NO: 48.

Expression cassette “fragment C” was prepared as described above in example 1 for example strain 1.

Expression cassette “fragment D” was prepared as described above in example 1 for example strain 1.

The above four cassettes were integrated in the intermediate strain in the INT59 locus on a non-coding region on chromosome XI, using CRISPR-Cas9 techniques as described above and the following sequences for homologous integration:
INT59_FLANK5 (illustrated by SEQ ID NO: 51); and
INT59_FLANK3 (illustrated by SEQ ID NO: 52)

Diagnostic PCR was performed to confirm the correct assembly and integration at the INT59 locus of the three expression cassettes. Plasmid free colonies were selected which resulted in example strain NX2 (see Table 2 for detailed genotypes).

### Example 3: Construction of Example Strain NX3

### (Yeast Strain Expressing Alpha 1,4-Glucosidase (Glucoamylase, EC 3.2.1.3), Alpha 1,6-Glucosidase (Debranching Glucoamylase, EC 3.2.1.10), Beta-Glucosidase (EC 3.2.1.21) and Alpha 1,4-Glucosidase (Trehalase, EC 3.2.1.28))

Example strain NX3 was constructed by transforming the intermediate strain IX1 mentioned above with four expression cassettes:
Expression cassette “fragment E”: 2J-Sc_PGK1.pro-Pstr_GA.orf_0009-Sc_ENO1.ter-2K
Expression cassette “fragment F”: 2K-Sc_RPS3.pro-Tcoc_GLA.orf-Sc_TDH1.ter-2L
Expression cassette “fragment G”: 2 L-Sc_ZUO1.pro-Akaw_BG17.orf-Sc_ADH1.ter-2M
Expression cassette “fragment H”: 2M-Sc_MYO4.pro-Tcel_Tre17.orf-Sc_AQR1.ter-2N

Expression cassette “fragment E” was prepared as described above in example 2 for example strain 2.

Expression cassette “fragment F” was prepared as described above in example 2 for example strain 2.

Expression cassette “fragment G”: The third cassette named “fragment G”, comprised the S. cerevisiae ZUO1 promoter (Sc_ZUO1.pro), Akaw_BG17.orf and S. cerevisiae ADH1 terminator (Sc_ADH1.ter). The cassette was decorated with 50 bp connectors 2L and 2M as illustrated in: SEQ ID NO: 40 and SEQ ID NO: 41, respectively. The nucleic acid sequence of the DNA fragment G is illustrated in SEQ ID NO: 49.

Expression cassette “fragment H”: The fourth cassette named “fragment H”, comprised the S. cerevisiae MYO4 promoter (Sc_MYO4.pro), Tcel_Tre17.orf and S. cerevisiae terminator (Sc_AQR1.ter). The cassette was decorated with 50 bp connectors 2M and 2N as illustrated in: SEQ ID NO: 41 and SEQ ID NO: 42, respectively. The nucleic acid sequence of the DNA fragment H is illustrated in SEQ ID NO: 50.

The above four cassettes were integrated in the intermediate strain in the INT59 locus on a non-coding region on chromosome XI, using CRISPR-Cas9 techniques as described above and the following sequences for homologous integration:
INT59_FLANK5 (illustrated by SEQ ID NO: 51); and
INT59_FLANK3 (illustrated by SEQ ID NO: 52)

Diagnostic PCR was performed to confirm the correct assembly and integration at the INT59 locus of the three expression cassettes. Plasmid free colonies were selected which resulted in example strain NX3 (see Table 2 for detailed genotypes).

### Example 4: Fermentations With Example Strains NX1, NX2 and NX3 and Comparative Strain A

Precultures of above Example strains NX1, NX2 and NX3 and comparative strain A were made as follows: Glycerol stocks (−80° C.) were thawed at room temperature and used to inoculate 0.2 L mineral medium (as described by Luttik, M L H. et al (2000) in their article titled “The Saccharomyces cerevisiae ICL2 Gene Encodes a Mitochondrial 2-Methylisocitrate Lyase Involved in Propionyl-Coenzyme A Metabolism”, published in J. Bacteriol. Vol. 182, pages 7007-7013) supplemented with 2% (w/v) glucose, at pH 6.0 (adjusted with 2M H2SO4/4N KOH), in non-baffled 0.5 L shake-flasks. The precultures were incubated for 16 to 20 hours at 32° C. and shaken at 200 RPM. After determination of the yeast cell dry weight (CDW) through OD600 measurement (using an existing CDW vs OD600 calibration line), a quantity of preculture corresponding to the required 0.5 gCDW/liter inoculum concentration for the propagation was centrifuged (3 min, 5300×g), washed once with one sample volume sterile demineralized water, centrifuged once more, and resuspended in propagation medium.

Propagations of above Example strains NX1, NX2 and NX3 and comparative strain A were carried out as follows: A propagation step was performed in 100 mL non-baffled shake flasks, using 20 mL diluted corn mash (70% v/v Corn mash: 30% v/v demineralized water) supplemented with 1.25 g/liter (L) urea (as nitrogen source) and an antibiotic mix (comprising 1 ml 100 μg/L penicillin G & 1 ml 50 μg/L Neomycin stock per liter of corn mash). After all additions, the pH was adjusted to 5.0 using 4N KOH/2M H2SO4. All strains were inoculated at 0.5 g CDW/L as described above and propagations for all strains were ran for 6 hrs at 32° C. shaking at 140 RPM. During propagation of comparative strain A external (ex-situ generated) glucoamylase (Spirizyme, commercially obtainable from Novozymes) was dosed at a dosage of 0.1 g/kg (i.e. 0.1 mL/L). During propagation of Example strains NX1, NX2 and NX3 no external (ex-situ generated) glucoamylase was dosed.

Main fermentations of above Example strains NX1, NX2 and NX3 and comparative strain A were carried out as follows: A main fermentation step was performed using 200 ml medium in 500 ml Schott bottles equipped with pressure recording/releasing caps (Ankom Technology, Macedon NY, USA), while shaking at 140 rpm and 32° C. pH was not controlled during fermentation. Fermentations were stopped after 66 h. Fermentations were executed with corn mash having dry solids (DS) content of about 33.4% w/w. Subsequently, the corn mash was supplemented with 1 g/L urea, and the antibiotics: neomycin and penicillin G to a final concentration of 50 μg/mL and 100 μg/mL (i.e. adding solutions 100 mg/ml PenG stock+50 mg/ml Neomycin stock respectively); antifoam (Basildon, approximately 0.5 mL/L). After all additions, the pH was adjusted to 5.0 using 2M H2SO4/4N KOH. The required yeast pitch from propagation to fermentation was 1.5% on fermentation volume. During the main fermentation of comparative strain A, external (ex-situ generated) alpha 1,4 glucosidase (glucoamylase, Spirizyme, commercially obtainable from Novozymes) was dosed at 0.24 g/kg (i.e. 0.24 mL/L). During the main fermentation of Example strains NX1, NX2 and NX3 no external (ex-situ generated) glucoamylase was dosed.

Sampling of the fermentation was carried out as follows: Samples were taken from the main fermentations only. Samples were taken at 18, 24, 42, 48, and 66 hours to assess effects of the expressed enzyme activities on sugar release profiles throughout the fermentation. The end of fermentation was at 66 hours. Since the fermentation broths contained active glucoamylase enzyme, 50 μl of a 10 g/L acarbose stock solution was added to approximately 5 g sample to stop glucoamylase activity. Samples for HPLC analysis were separated from yeast biomass and insoluble components (corn mash) by passing the clear supernatant after centrifugation through a 0.2 μm pore size filter. HPLC (Aminex) analysis was conducted.

Conclusions were as follows: Residual sugars (g/L), ethanol and glucose concentrations in the fermentation broth were measured during fermentation at 18 hours, 48 hours and at the end of fermentation (66 hours) by HPLC. The results are summarized in Table 3 below.

It was found that in the experiments with the strains according to the invention, where the glucosidase were produced in-situ and no external ex-situ produced glucosidase were added, resulted in less residual sugar at the end of fermentation (i.e. at 66 hours). In addition the ethanol yields at the end of fermentation were higher and less glucose remained unconverted.

These results illustrate that with the process and recombinant yeast cells according to the invention advantageously not only comparable but even better results can be obtained, even without the addition of external ex-situ produced glucosidase.

TABLE 3
Residual sugars at the end of fermentation
(66 hours) measured by HPLC (mg/L)

| | Time | Total sugar | ethanol | glucose |
|---|---|---|---|---|
| Strain | (hour) | (g/L) | (g/L) | (g/L) |
| Example strain NX1 | 18 | 145.97 | 66.80 | 73.60 |
| Example strain NX1 | 48 | 11.57 | 135.40 | 0.92 |
| Example strain NX1 | 66 | 10.80 | 139.30 | 0.57 |
| Example strain NX2 | 18 | 156.00 | 64.00 | 52.60 |
| Example strain NX2 | 48 | 11.15 | 137.00 | 0.75 |
| Example strain NX2 | 66 | 10.22 | 138.20 | 0.42 |
| Example strain NX3 | 18 | 148.27 | 63.40 | 55.60 |
| Example strain NX3 | 48 | 11.17 | 136.10 | 0.69 |
| Example strain NX3 | 66 | 10.64 | 141.00 | 0.54 |
| Comparative strain A* | 18 | 138.32 | 69.80 | 76.60 |
| Comparative strain A* | 48 | 10.82 | 133.70 | 0.76 |
| Comparative strain A* | 66 | 11.22 | 136.10 | 1.49 |

*During the fermentations with Comparative strain A, external (ex-situ produced) alpha 1,4 glucosidase (glucoamylase, Spirizyme, commercially obtainable from Novozymes) was dosed at 0.24 g/kg (i.e. 0.24 mL/L).

1. A process for the production of ethanol, comprising:
fermentation of a feed, under anaerobic conditions, wherein the feed contains a di-saccharide, oligo-saccharide and/or poly-saccharide and wherein the fermentation is carried out in the presence of a recombinant yeast cell, which recombinant yeast produces a combination of proteins having glucosidase activity; and
recovery of ethanol.

2. The process according to claim 1, wherein the process further comprises dosing of an ex-situ produced protein having glucosidase activity at a concentration of 0.05 g/L or less, calculated as the total amount of such protein in grams per litre of feed.

3. The process according to claim 1 or 2, wherein no ex-situ produced protein having glucosidase activity is dosed during fermentation.

4. The process according to any one of claims 1 to 3, wherein the total weight percentage of di-saccharide, oligo-saccharide and poly-saccharide, based on the total weight of saccharides present in the feed, is equal to or more than 1% w/w, preferably equal to or more than 5% w/w, more preferably equal to or more than 10% w/w and most preferably equal to or more than 20% w/w.

5. The process according to any one of claims 1 to 4, wherein the feed contains
a first di-saccharide, oligo-saccharide and/or poly-saccharide consisting of two or more mono-saccharide units linked to each other via an alpha-1,4-glycosidic bond; and
a further di-saccharide, oligo-saccharide and/or poly-saccharide containing at least two mono-saccharide units linked to each other via an alpha-1,6-glycosidic bond, an alpha-1,1-glycosidic bond or a beta-glycosidic bond.

6. The process according to any one of claims 1 to 5, wherein the recombinant yeast cell is a recombinant Saccharomyces yeast cell

7. The process according to any one of claims 1 to 6, wherein the recombinant yeast cell produces a combination of:
a first protein having alpha 1,4-glucosidase activity (E.C. 3.2.1.3); and
a further protein having alpha 1,6-glucosidase activity (E.C. 3.2.1.10); and/or a further protein having beta-glucosidase activity (E.C. 3.2.1.21); and/or a further protein having alpha 1,1-glucosidase activity (E.C. 3.2.1.28).

8. The process according to any one of claims 1 to 7, wherein the recombinant yeast cell produces a combination of:
a first protein having alpha 1,4-glucosidase activity (E.C. 3.2.1.3); and
a further protein having alpha-1,6-glucosidase activity (E.C. 3.2.1.10); and a further protein having beta-glucosidase activity (E.C. 3.2.1.21); and a further protein having alpha 1,1-glucosidase activity (E.C. 3.2.1.28).

9. The process according to any one of claims 1 to 8, wherein the recombinant yeast cell further produces:
a protein comprising phosphoketolase activity (EC 4.1.2.9 or EC 4.1.2.22); and/or
a protein having phosphotransacetylase (PTA) activity (EC 2.3.1.8); and/or
a protein having acetate kinase (ACK) activity (EC 2.7.2.12); and/or
a protein having ribulose-1,5-biphosphate carboxylase oxygenase (Rubisco) activity; and/or
a protein having phosphoribulokinase (PRK) activity; and/or
a protein comprising NADH dependent acetylating acetaldehyde dehydrogenase activity; and/or
a protein comprising acetyl-CoA synthetase activity; and/or
a protein comprising alcohol dehydrogenase activity; and/or
a protein having glycerol dehydrogenase activity (E.C. 1.1.1.6); and/or
a protein having dihydroxyacetone kinase activity (E.C. 2.7.1.28 or E.C. 2.7.1.29); and/or
a protein having glycerol transporter activity.

10. The process according to any one of claims 1 to 9, wherein the process comprises an enzymatic hydrolysis step and a fermentation step, wherein the enzymatic hydrolysis step is carried out separately from the fermentation step.

11. The process according to any one of claims 1 to 9, wherein the process comprises an enzymatic hydrolysis step and a fermentation step, wherein both steps are carried out simultaneously.

12. A recombinant Saccharomyces yeast cell functionally expressing:
a first nucleotide sequence encoding a first protein having alpha-1,4-glucosidase activity; and
a further nucleotide sequence encoding a further protein having a glucosidase activity other than an alpha-1,4-glucosidase activity.

13. A recombinant Saccharomyces yeast cell according to claim 10, functionally expressing:
a first nucleotide sequence encoding a first protein having alpha-1,4-glucosidase activity; and
a further nucleotide sequence encoding a further protein having alpha-1,6-glucosidase activity and/or a further nucleotide sequence encoding a further protein having alpha-1,1-glucosidase activity and/or a further nucleotide sequence encoding a further protein having beta-glucosidase activity.

14. The recombinant Saccharomyces yeast cell according to claim 10 or 11, further functionally expressing:
a nucleotide sequence encoding a protein comprising phosphoketolase activity (EC 4.1.2.9 or EC 4.1.2.22); and/or
a nucleotide sequence encoding a protein having phosphotransacetylase (PTA) activity (EC 2.3.1.8); and/or
a nucleotide sequence encoding a protein having acetate kinase (ACK) activity (EC 2.7.2.12); and/or
a nucleotide sequence encoding a protein having ribulose-1,5-biphosphate carboxylase oxygenase (Rubisco) activity; and/or
a nucleotide sequence encoding a protein having phosphoribulokinase (PRK) activity; and/or
a nucleotide sequence encoding a protein comprising NADH dependent acetylating acetaldehyde dehydrogenase activity; and/or
a nucleotide sequence encoding a protein comprising acetyl-CoA synthetase activity; and/or
a nucleotide sequence encoding a protein comprising alcohol dehydrogenase activity; and/or
a nucleotide sequence encoding a protein having glycerol dehydrogenase activity (E.C. 1.1.1.6); and/or
a nucleotide sequence encoding a protein having dihydroxyacetone kinase activity (E.C. 2.7.1.28 or E.C. 2.7.1.29); and/or
a nucleotide sequence encoding a protein having glycerol transporter activity.

15. The process according to any one of claims 1 to 11, wherein the recombinant yeast cell is the Saccharomyces yeast cell according to anyone of claims 12 to 14.
